(19) United States
(12) Patent Application Publication
(10) Pub. No.: US 2013/0210034 A1
(43) Pub. Date: Aug. 15, 2013

(54) COMPLEX PHOSPHOPROTEIN ACTIVATION PROFILES

(71) Applicant: Beckman Coulter, Inc., (US)

(72) Inventors: James Jacobberger, Novelty, OH (US); Charles Goolsby, Winfield, IL (US); David Hedley, Toronto (CA); Vincent Shankey, Miami, FL (US); Phillip Woost, North Ridgeville, OH (US); Sue Chow, Markham (CA); James Marvin, Evanston, IL (US)

(73) Assignee: BECKMAN COULTER, INC., Brea, CA (US)

(21) Appl. No.: 13/781,312

(22) Filed: Feb. 28, 2013

Related U.S. Application Data
(63) Continuation-in-part of application No. 12/696,702, filed on Jan. 29, 2010.
Application No. 12/696,702 is a continuation of application No. 11/267,948, filed on Nov. 4, 2005, now abandoned.

(51) Int. Cl. G01N 33/68

(52) U.S. Cl. CPC G01N 33/6893
USPC 435/7.23; 435/7.21

(57) ABSTRACT

The present specification discloses methods for determining a phosphoprotein activation profile in hematopoietic cells, methods for detecting a signal transduction activation state in an individual having or suspected of having a disease or condition associated with activation of a signal transduction pathway, methods for detecting leukemia, and kits for determining a phosphoprotein activation profile in a sample containing hematopoietic cells.

### CROSS-REFERENCE

This application is a continuation-in-part that claims priority pursuant to 35 U.S.C. §120 to U.S. patent application Ser. No. 12/696,702, filed Jan. 29, 2010, a continuation application of U.S. patent application Ser. No. 11/267,948, filed Nov. 4, 2005, and claims priority to International Patent Application Serial No. PCT/US2011/049596, filed Aug. 29, 2011, an application that claims priority pursuant to 35 U.S.C. §119(e) to U. S. Provisional Patent Application Ser. No. 61/378,258, filed Aug. 30, 2010 and to U.S. Provisional Patent Application Ser. No. 61/378,246, filed Aug. 30, 2010, each of which is hereby incorporated by reference in its entirety.

### BACKGROUND OF THE INVENTION

Stem Cell Factor (SCF), FMS-Like Tyrosine Kinase 3 (FLT-3) ligand (FL), Interleukin- 3 (IL-3), Granulocyte Colony-Stimulating Factor (G-CSF), and Granulocyte-Macrophage Colony-Stimulating Factor (GM-CSF) are among the major cytokines that regulate hematopoiesis. They share a number of common properties, including autocrine/paracrine regulation, overlapping and redundant functionality, synergy in combination with other cytokines, and activation of similar signal transduction mechanisms. However, each cytokine has a distinct range of regulatory activity on the hematopoietic system, regulation that is mediated by a unique membrane-bound receptor.

SCF binds to KIT (synonymous with CD117), a receptor tyrosine kinase (RTK), that belongs to the same class as the platelet-derived growth factor receptor (i.e., class III). KIT is expressed on HSCs, on common myeloid and lymphoid progenitors, and on more committed progenitors in each of these lineages, including those of the monocytic and neutrophilic lines; but, in general, expression of Kit is lost as cells fully differentiate. Ligand binding results in receptor dimerization, autophosphorylation, and continued transduction of the SCF signal through multiple downstream pathways, including the Phosphatidylinositol 3-Kinase-AKT (PI3K-AKT) pathway, a mammalian target of rapamycin (mTOR) pathway, and the Rat Sarcoma-Mitogen Activated Protein Kinase (RAS-MAPK) pathway. Consequently, SCF and KIT, along with other intra- and extracellular effectors, are thought to play an important role in normal hematopoiesis, including proliferation, differentiation, and survival.

FL binds to FLT-3. Like KIT, FLT-3 is a class III RTK, and is expressed on committed myeloid and lymphoid progenitors as well as some more mature cells in the monocytic lineage. FL is expressed primarily by stromal cells of the bone marrow in either soluble or membrane-bound forms. Together, binding of FL to FLT3, like the binding of SCF to Kit, leads to activation of several downstream mediators, including the PI3K-AKT pathway, the mTOR pathway, the RAS-MAPK pathway, and additionally the Janus Kinase-Signal Transducer and Activator of Transcription, (JAK-STAT) pathway, ultimately affecting proliferation, differentiation, survival, and apoptosis.

IL-3, G-CSF, and GM-CSF belong to a group of cytokines called colony-stimulating factors. Their receptors belong to the pg140 family, which is characterized by a unique ligand binding α-subunit and a common signal transduction β-subunit. IL-3, G-CSF, and GM-CSF receptors are expressed on a variety of cell types including CD34⁺ as well as primitive and committed hematopoietic progenitor cells. IL-3 stimulates the differentiation of multipotent hematopoietic stem cells into myeloid progenitor cells (as opposed to lymphoid progenitor cells where differentiation is stimulated by IL-7) as well as stimulates proliferation of all cells in the myeloid lineage (erythrocytes, megakaryocytes, granulocytes, monocytes, and dendritic cells). Similarly, both G-CSF and GM-CSF function as white blood cell growth factors by stimulating bone marrow stem cells to produce granulocytes (neutrophils, eosinophils, and basophils), macrophages, megakaryocytes, and erythroid cells and monocytes. Whereas IL-3 broadly targets hematopoietic stem cells and the earliest progenitors regulating the growth, differentiation, and survival of neutrophils, eosinophils, basophils, mast cells, mactophages, megakaryocytes, and erythroid cells. G-CSF and GM-CSF function at a slightly more mature state of differentiation. Signaling is achieved through multiple pathways, including the PI3K-AKT pathway, the mTOR pathway, the RAS-MAPK pathway, and the JAK-STAT pathway.

Importantly, it appears that misregulation of hematopoiesis in any these cytokines may be linked to various hematopoetic disease or condition including, without limitation acute myelogenous leukemia (AML), an acute lymphocytic leukemia (ALL), a chronic lymphocytic leukemia (CLL), a lymphoma, a follicular lymphoma, or a multiple myeloma. In contrast to the normal phenotype, hematopoetic diseases are characterized by unregulated proliferation, impaired differentiation of hematopoietic progenitors into mature blood cells, and increased leukemic blast survival. In part, these dysfunctions are related to deregulation of critical signal transduction pathways and loss of the normal cellular differentiation program. In the majority of hematopoetic diseases, deregulated signaling has been attributed to altered signaling through KIT and FLT-3 cell surface receptor tyrosine kinases, through increased expression, gain-of-function mutations, and through autocrine/paracrine stimulation. For example, dysfunctions in KIT expression or activation are linked to a number of hematopoetic diseases, including AML. For example, gain-of-function mutations in the KIT receptor are also thought to be involved in the etiology of AML and other human cancers. Similarly, mutations in the gene encoding FLT-3 are among the most common in AML patients. In addition, 15% to 35% of AML patients express internal tandem duplications in the Flt-3 gene, and 5% to 10% express activation loop mutations, which result in constitutive receptor activation, inappropriate FLT-3 signaling, and mutation-related biologic dysfunction. Furthermore, 25% to 45% of AML patients have at least one of these FLT-3 mutations, making them among the most common genetic abnormalities in AML. These dysfunctions result in altered phosophorylation states of proteins from these pathways including, without limitation, Extracellular-Signal-Regulated Kinase ½ (ERK½), AKT, ribosomal S6 protein (S6), STAT1, STAT3, STAT5, STATE, and p38 MAPK.

Thus, the ability to monitor the activation profile of the signaling pathways associated with hematopoiesis, including the phosophorylation states of proteins from these pathways, would be useful as a diagnostic tool for diseases or conditions associated with these pathways. Unfortunately, previous studies utilizing immunoblotting techniques have limited effectiveness in measuring interactions between signaling pathways, and do not provide meaningful data measuring the response of single cells in a total cell population, particularly where the target population of cells exists as a low percentage of the total cell population. In addition, assays relying on immunoblotting techniques cannot identify the actual cell type generating the response in a mixed cell population. Furthermore, these techniques require that the population be treated before measurement to enrich the population for the target cells, introducing artifacts from activation of signaling pathways caused by the enriching processes required and introducing difficulties in knowing what target population should be enriched for. However, for hematopoietic diseases, characterizing the kinetics of baseline phosphoprotein activation profiles in normal, healthy tissue is essential, in order to fully understand both the major differences as well as the fine distinctions observed in the diseased state.

A need in the art exists for an assay that can accurately measure the profiles of phosphoprotein activation in a sample containing hematopoietic cells. Other needs exist for methods for monitoring hematopoietic diseases or conditions that are associated with aberrant activation of a signal transduction pathway, including leukemias. Needs exist for assays that can effectively compare the kinetic profiles of activation of phosphoproteins in normal samples to the corresponding activation profiles in diseased samples. Sensitive assays are especially needed in patients in which obtaining a sample is difficult, for example, bone marrow.

### BRIEF SUMMARY

Combining the single-cell resolution of multiparametric flow cytometry with high affinity, fluorophore-conjugated monoclonal antibodies, the present specification discloses methods to simultaneously measure both surface biomarkers and intracellular signaling proteins, typically, phosphoproteins, in multiple, rare, cell subpopulations of hematopoietic cells in both healthy individuals or individuals suffering from a hematopoietic disease or condition like an AML, an ALL, a CLL, a lymphoma, a follicular lymphoma, or a multiple myeloma. The present specification discloses that by using hematopoietic cytokines to activate the PI3K-AKT, RAS-MAPK, and/or JAK-STAT signal transduction pathways, detailed kinetic profiles of phosphorylated S6, ERK, AKT, STAT3, and/or STAT5 in primitive hematopoietic progenitor cells were obtained. Comparison to analogous profiles from patients with AML showed distinct, pronounced differences in the phosphoprotein profiles useful for diagnostic evaluation and/or therapeutic advantage.

Aspects of the present specification disclose methods for determining a phosphoprotein activation profile in hematopoietic cells, the methods comprising the steps of a) incubating a test sample comprising hematopoietic cells with a phosphoprotein activator for at least a first incubation time period and a second incubation time period, wherein the hematopoietic cells comprise a phosphoprotein of at least one signal transduction pathway; and wherein the phosphoprotein activator is capable of activating the phosphoprotein of at least one signal transduction pathway present in the hematopoietic cells of the test sample; b) contacting the test sample comprising hematopoietic cells incubated for at least a first incubation time period and a second incubation time period with one or more fluorescently labeled capture molecules, the one or more fluorescently labeled capture molecules comprising at least one fluorescently labeled activated phosphoprotein capture molecule capable of binding to the phosphoprotein of at least one signal transduction pathway activated by the phosphoprotein activator; and c) detecting fluorescence of the one or more fluorescently labeled capture molecules from test sample comprising hematopoietic cells incubated for at least a first incubation time period and a second incubation time period; wherein the fluorescence of the at least one fluorescently labeled activated phosphoprotein capture molecule detected for the first incubation time period and the fluorescence of the at least one fluorescently labeled activated phosphoprotein capture molecule detected for the second incubation time period determines the phosphoprotein activation profile in a test sample comprising hematopoietic cells. In some aspects, the measurement of the kinetics of phosphoprotein activation over time in certain cell subtypes within a complex cell population is desirable. This is important where a comparison of such activation profiles between normal and diseased samples can identify cells in a sample population representing a diseased state. This is additionally important where patient samples are difficult to obtain, for example, bone marrow samples.

Other aspects of the present specification disclose methods for detecting leukemia, the methods comprising the steps of a) incubating a test sample comprising hematopoietic cells and a reference sample comprising hematopoietic cells with a phosphoprotein activator, wherein the test sample is obtained from an individual having or suspected of having a leukemia; wherein the reference sample is obtained from an individual not having or not suspected of having a leukemia; wherein the hematopoietic cells of the test sample and the reference sample comprise a phosphoprotein of at least one signal transduction pathway; and wherein the phosphoprotein activator is capable of activating the phosphoprotein of at least one signal transduction pathway present in the hematopoietic cells of the test sample; b) contacting the test sample comprising hematopoietic cells and a reference sample comprising hematopoietic cells with one or more fluorescently labeled capture molecules, wherein the one or more fluorescently labeled capture molecules comprise at least one fluorescently labeled activated phosphoprotein capture molecule capable of binding to the phosphoprotein of at least one signal transduction pathway activated by the phosphoprotein activator; c) detecting fluorescence of the one or more fluorescently labeled capture molecules present in the test sample and the reference sample; and d) comparing the fluorescence detected for the test sample comprising hematopoietic cells to the fluorescence detected for the reference sample comprising hematopoietic cells, wherein a difference in the fluorescence detected for the test sample comprising hematopoietic cells relative to the fluorescence detected for the reference sample comprising hematopoietic cells is indicative of the leukemia.

Yet other aspects of the present specification disclose methods for detecting a signal transduction activation state in an individual having or suspected of having a disease or condition associated with activation of a signal transduction pathway, the methods comprising the steps of a) determining a phosphoprotein activation profile of at least one signal transduction pathway from a hematopoietic cell population in a test sample, the test sample obtained from an individual having or suspected of having a disease or condition associated with activation of a signal transduction pathway; b) determining a phosphoprotein activation profile of at least one signal transduction pathway from a hematopoietic cell population in a reference sample, the reference sample obtained from an individual not having or not suspected of having a disease or condition associated with activation of a signal transduction pathway, wherein the phosphoprotein activation profile of at least one signal transduction pathway measured from the test sample and the reference sample is the same; and c) comparing the phosphoprotein activation profile measured in step (a) with the phosphoprotein activation profile measured in step (b), wherein identifying a difference in the phosphoprotein activation profile measured in step (a) from the phosphoprotein activation profile measured in step (b) is indicative of the disease or condition associated with activation of a signal transduction pathway.

Still other aspects of the present specification disclose methods for detecting a leukemia, the methods comprising the steps of a) determining a phosphoprotein activation profile of at least one signal transduction pathway from a hematopoietic cell population in a test sample, the test sample obtained from an individual having or suspected of having a leukemia; b) determining a phosphoprotein activation profile of at least one signal transduction pathway from a hematopoietic cell population in a reference sample, the reference sample obtained from an individual not having or not suspected of having a leukemia, wherein the phosphoprotein activation profile of at least one signal transduction pathway measured from the test sample and the reference sample is the same; and c) comparing the phosphoprotein activation profile measured in step (a) with the phosphoprotein activation profile measured in step (b), wherein identifying a difference in the phosphoprotein activation profile measured in step (a) from the phosphoprotein activation profile measured in step (b) is indicative of the leukemia.

Aspects of the present specification disclose kits for determining a phosphoprotein activation profile in a sample containing hematopoietic cells, the kits comprising: a)a cytokine activator of a PI3K-AKT pathway, a mTOR pathway, a RAS-MAPK pathway, a JAK-STAT pathway, or any combination thereof; b) a CD34 capture molecule; c) a CD117 capture molecule; and d) one or more of phosphoprotein capture molecules, the one or more phosphoprotein capture molecules comprising a pS6 capture molecule, a pERK capture molecule, a pAKT capture molecule, a pSTAT3 capture molecule, a pSTAT5 capture molecule, or any combination thereof.

### BRIEF DESCRIPTION OF THE FIGURES

The accompanying figures, which are incorporated herein and form a part of the specification, illustrate one or more embodiments of the invention and, together with the description, further serve to explain the principles of the invention.

FIG. 1 shows a gating strategy.

FIG. 2 shows dysregulation highlighting loss of pERK response to SCF stimulation in the CD34+ cell population in an AML patient.

FIG. 3 shows kinetic differences in pAKT upregulation between samples from normal individuals and AML patients.

FIG. 4 shows a gating strategy.

FIG. 5 shows composite profiles for SCF-, FL-, IL-3-, and GM-CSF-stimulated phosphorylation of cell populations obtained from bone marrow samples collected from normal healthy donor individuals.

FIG. 6 shows inhibition of SCF-stimulated pERK (A) and pS6 (B), lack of SCF response in lymphocytes from bone marrow samples of healthy donors (C and D), FL response in lymphocytes from bone marrow samples of healthy donors (E and F), and GM-CSF stimulated pS6, pERK, and pSTAT5 in monocytes from bone marrow samples of healthy donors(G and H).

FIG. 7 shows the stability of SCF-stimulated pERK signaling (FIG. 7A) and the stability of SCF-stimulated pAKT signaling (FIG. 7B) in CD34⁺/117⁺ cell populations obtained from bone marrow samples of healthy donors.

FIG. 8 shows cytokine-stimulated phosphorylation profiles of pS6, pERK, and pSTAT5 cell populations obtained from bone marrow samples of AML1 and AML2. Results are expressed as fold stimulation.

FIG. 9 shows a comparison of phosphoprotein profiles from healthy donor and AML bone marrow samples. Phosphoprotein-specific comparisons were made showing SCF-stimulated pERK and pS6 in healthy donor, AML1, and AML2, FL-stimulated pERK and pS6 in healthy donor and AML1, IL-3-stimulated pSTAT5 in healthy donor and AML1, and GM-CSF-stimulated pSTAT5 in NBM and AML1. For healthy donor, the data are represented as the mean ±95% CI. Results are expressed as fold stimulation.

FIG. 10 shows a comparison of data obtained from SCF-stimulated phosphorylation of pS6 and pERK in cell populations derived from healthy donor and AML bone marrow samples by evaluating mean fluorescent intensity (MFI)(FIG. 10A), frequency (FIG. 10B), positives over negatives (FIG. 10C), and fold stimulation (FIG. 10D).

FIG. 11 shows the difference in signaling amplitude and duration of SCF-stimulated phosphorylation of pS6 and pERK measured in healthy donor samples (composite data obtained from 9 healthy donors; grey) versus data obtained from 3 different AML bone marrow samples (AML1; red), (AML2; green), and (AML3; blue).

FIG. 12 shows two views (FIG. 12A and FIG. 12B) of the difference in signaling amplitude and duration of SCF-stimulated phosphorylation of pS6 and pERK measured in healthy donor samples (composite data obtained from 9 healthy donors; grey) versus data obtained from 5 different AML bone marrow samples (AML1; red), (AML2; green), (AML3; blue), (AML4; aqua), and (AML5; violet) as well as one view (FIG. 12C) of the difference in signaling amplitude and duration of FL-stimulated phosphorylation of pS6 and pERK measured in healthy donor samples (composite data obtained from 9 healthy donors; grey) versus data obtained from 3 different AML bone marrow samples (AML1; red), (AML3; blue), and (AML5; violet).

### DETAILED DESCRIPTION

The methods disclosed herein are typically performed in vitro on a sample or test sample. The terms test sample and sample are used interchangeably herein. The sample in the methods of the present invention can include any hematopoietic cell-containing sample or any white blood cell-containing sample, including a bone marrow sample, for example, aspirated bone marrow samples, and orthopedic surgery bone specimens. In other embodiments, the sample is a needle aspirate such as a lymph node aspirate, or a clinical sample containing a cell suspension. In a preferred embodiment, the sample is a bone marrow sample. The bone marrow sample can be obtained from the individual or test subject using standard clinical procedures.

Obtaining a bone marrow sample encompasses obtaining the bone marrow directly from an individual, for example from a donor, volunteer or patient. Obtaining a bone marrow sample also encompasses obtaining a bone marrow sample that was previously obtained from a patient, for example a laboratory technician obtaining a patient's bone marrow sample for analysis using the methods of the present invention. Bone marrow can be obtained by aspiration from an individual's bone tissue by a trained physician, for example from the posterior ileac crest. Such a process provides a population of bone marrow cells, including white blood cells, CD34+, CD117+ cells, bone marrow blast cells, and red blood cells.

Samples can be obtained from a human person or a commercially significant mammal, including but not limited to a cow or horse. Samples can also be obtained from household pets, including but not limited to a dog or cat.

In some embodiments, the sample is obtained from normal bone marrow, i.e. from healthy, adult donors. In certain embodiments, the sample is obtained from diseased bone marrow, from an individual having a disease affecting signal transduction pathway activation or activation of phosphoproteins in bone marrow cells. In one embodiment, the disease is AML. In some embodiments, samples from normal individuals are used as controls to correlate signal transduction pathway activity or activation of phosphoproteins. The bone marrow is the site where AML “stem cells” reside and proliferate. In general, it is believed that these marrow “stem cells” are found in the peripheral blood either when the marrow becomes crowded with leukemic cells, or when the marrow “stem cells” are “mobilized” by in vivo treatment with specific cytokines (e.g., G-CSF) or with compounds which inhibit the stem cell surface receptors from binding to contra-receptors which normally attract them to the bone marrow “niche” (e.g., CXCR4 receptor on bone marrow stem cells normally binds to SDF-1 in the “niche”, maintaining stem cells at this site). Since peripheralized AML stem cells are not in their preferred environment, it is likely they change their biologic characteristics in blood versus in the bone marrow.

As used herein, a “phosphoprotein” refers to a protein that has at least one isoform (and in some cases two or more isoforms) that corresponds to a specific form of the protein having a particular biological, biochemical, or physical property, e.g., an enzymatic activity, a modification (e.g., post-translational modification), or a conformation. The phosphoprotein can be activated or unactivated (i.e., non activated) with respect to a particular biological activity, modification, or conformation. Specifically, the activated or active form of the phosphoprotein has the particular biological activity, modification, or conformation, whereas the unactivated or unactive (non-active) form of the phosphoprotein does not have (or has a lesser or diminished level of) the particular biological activity, modification, or conformation, respectively. In some embodiments, there can be more than one isoform associated with an activity or activation state; for example, there can be an isoform associated with an “open” conformation available for substrate binding, a second transition state isoform, and an isoform devoid of activity (e.g., where the activity is inhibited). In certain embodiments, the phopsphoprotein is a protein that exists in phosphorylated form when it is activated and non-phosphorylated form when it is not activated. Examples of phosphoproteins include, without limitation, ERK and its phosphorylated form pERK, AKT and its phosphorylated form pAKT, S6 and its phosphorylated form pS6, STAT1 and its phosphorylated form pSTAT1, STAT2 and its phosphorylated form pSTAT2, STAT3 and its phosphorylated form pSTAT3, STAT4 and its phosphorylated form pSTAT4, STAT5 and its phosphorylated form pSTAT5, and STAT6 and its phosphorylated form pSTAT6.

In a certain embodiment, the biological, biochemical, or physical property (e.g. enzymatic activity, modification, or conformation) of the phosphoprotein can be induced, stimulated, or activated by a phosphoprotein activator or by cell signaling events initiated by a phosphoprotein activator. Examples of phosphoprotein activators include, without limitation, cytokines, kinases, phosphatases, proteases (e.g., caspases), and hormones. In some embodiments, a phosphoprotein activator includes SCF, FL, IL-3, G-CSF, GM-CSF, or any combination thereof. Examples of cell signaling events include, but are not limited to, receptor clustering or binding of a cognate molecule or ligand.

As used herein, an isoform refers to a form of a phosphoprotein having a specific, and preferably detectable, biological activity, modification, or conformation, or lack thereof, i.e., the isoform can be an activated (or active) form, or unactivated (or not active) form of a phosphoprotein. As mentioned, in certain embodiments, the binding of an activated phosphoprotein capture molecule to a corresponding isoform of an activated phosphoprotein is indicative of the identity of the phosphoprotein in its active state. In a certain embodiment, the invention provides methods for determining a phosphoprotein activity profile which comprise determining the presence of an activated isoform of a phosphoprotein (or activated phosphoprotein).

In a certain embodiment, the activated phosphoprotein is an isoform of the phosphoprotein having a particular or specific biological, biochemical, or physical property that is not possessed by at least one other isoform of the phosphoprotein. Examples of such properties include, but are not limited to, enzymatic activity (e.g., kinase activity and protease activity), and receptor binding activity. Thus, such particular or specific properties or activities are associated with an activated phosphoprotein isoform.

An example of activated phosphoprotein is a phosphoprotein having protein kinase activity. For example, a signal transduction pathway phosphoprotein with protein kinase activity refers to signal transduction pathway phosphoprotein that when activated is capable of catalyzing the phosphorylation of amino acids, or derivatives thereof, which possess a hydroxyl group. Preferred kinases are those that are capable of catalyzing the phosphorylation of serine, threonine, and tyrosine residues. Kinase activity can be determined by supplying a substrate for phosphorylation by kinase, a source of phosphate usable by kinase, and determining the phosphorylation of substrate in the presence of kinase.

The antigenicity of an activated phosphoprotein is distinguishable from the antigenicity of non-activated phosphoprotein isoform or from the antigenicity of a phosphoprotein isoform of a different activation state. In a certain embodiment, an activated phosphoprotein possesses an epitope that is absent in a non-activated phosphoprotein isoform, or vice versa. In another embodiment, this difference is due to covalent addition of moieties to a phosphoprotein, such as phosphate moieties, or due to a structural change in a phosphoprotein, as through protein cleavage, or due to an otherwise induced conformational change in a phosphoprotein which causes the phosphoprotein to present the same sequence in an antigenically distinguishable way. In another embodiment, such a conformational change causes an activated phosphoprotein to present at least one epitope that is not present in a non-activated phosphoprotein isoform, or to not present at least one epitope that is presented by an unactivated (i.e., non-activated) isoform of the phosphoprotein. In some embodiments, the epitopes for the distinguishing capture molecules are centered around the active site of the phosphoprotein, although as is known in the art, conformational changes in one area of a phosphoprotein can cause alterations in different areas of the phosphoprotein as well.

In certain embodiments, the signal transduction pathway is the PI3K-AKT pathway, the mTOR pathway, the RAS-MAPK pathway, or the JAK-STAT pathway. The MAPK pathway is a signal transduction pathway that effects gene regulation, and which controls cell proliferation and differentiation in response to extracellular signals. This pathway is also involved in oocyte meiotic maturation. The MAPK pathway is found, e.g., in frogs, and in mammals, e.g., mice, rats, and humans. This pathway can be activated by cytokines such as IL-1 and TNF, and constitutively activated by proteins such as MOS, RAF, RAS, and V12HARAS. The PI3K pathway mediates and regulates cellular apoptosis. The PI3K pathway also mediates cellular processes, including proliferation, growth, differentiation, motility, neovascularization, mitogenesis, transformation, viability, and senescence. The cellular factors that mediate the PI3K pathway include PI3K, AKT, and BAD. These factors mediate and regulate cellular apoptosis. The PI3K factors include class I PI3K, a cytosolic enzyme complex which includes p85 and p110. BAD has been identified as a pro-apoptotic member of the bcl-2 family.

The mTor (mammalian target of rapamycin) protein is activated by upstream AKT/PKB, and as such, is part of the P13 Kinase signaling pathway. Activated mTor regulates cell growth and homeostasis through several downstream pathways, including p7ORSK, 4EBP1 and eIF4B. mTor functions as an ATP and amino acid “sensor” to balance nutrient availability and cell growth in normal cells, and deregulation of these functions are commonly found in cancer cells. In particular embodiments, the phosphoprotein of a signal transduction pathway that is activated is S6, ERK, STAT5, or AKT or combinations thereof.

The JAK-STAT pathway mediates signaling by specific cytokines and growth factors (e.g. G-CSF and GM-CSF) and their cell surface receptors. JAK proteins associate with cytokine receptors and upon binding of cytokine to its surface receptor, JAK proteins become phosphorylated at specific amino acid residues to provide binding sites for multiple signaling proteins, including STATs. Upon activation by JAK, phosphorylated STATs dimerize and translocate to the nucleus, bind to specific DNA sequences, and transcriptionally activate specific genes.

Ribosomal S6 protein belongs to S6E family of ribosomal proteins and it is involved in the control of cell growth and proliferation via selective translation (Molina H. et al.; PNAS. USA 104: 2199-2204, (2007)). It is a major substrate of Ribosomal Protein S6 Kinase (RSK) in the eukaryote ribosomes. During translation, it regulates the translation of any RNA which contains 5′ terminal oligopyrimidine sequence (5′TOP). 5′TOP encodes proteins for cell cycle progression, ribosomal proteins, and elongation factors. The phosphorylation of S6 has been linked to increase in selective 5′TOP translation. The major phosphorylation sites in S6 includes Ser235, 236, 240, and 244. The phosphorylation of S6 is stimulated by growth factors, tumor promoting agents, and mitogens. During growth arrest, S6 is dephosphorylated.

In certain embodiments, a phosphoprotein of at least one signal transduction pathway is AKT, PI3K, S6, p44/42 MAP kinase, TYK2, p38 MAP kinase, PKC, PKA, SAPK, ELK, JNK, cJun, RAS, Raf, MEK ½, MEK 3/6, MEK 4/7, ZAP-70, LAT, SRC, LCK, ERK ½, Rsk 1, PYK2, SYK, PDK1, GSK3, FKHR, AFX, PLCg, PLCy, FAK, CREB, αIIIβ3, FcεRI, BAD, p70S6K, STAT1, STAT2, STAT3, STAT5, STAT6, or combination of these proteins.

In certain embodiments, the sample is incubated with a phosphoprotein activator or one or more phosphoproteins disclosed herein. In certain embodiments, the phosphoprotein activator is SCF, FL, IL-3, G-CSF, GM-CSF or combinations thereof. Optimal incubation times and temperatures for each sample preparation step can be readily determined using routine experimentation. In one embodiment, activation by the cytokine activator can be performed for about 0.5 minutes to about 60 minutes. Alternatively, activation can be performed for about 0.5 minutes to about 30 minutes.

During this incubation, activation of a phosphoprotein of at least one signal transduction pathway can be monitored at various times to determine maximal response, interval of response, and amplitude of response. This can be done by removing aliquots from the sample at various times during incubation, such as e.g., a first incubation time period, a second incubation time period, a third incubation time period, a fourth incubation time period, a fifth incubation time period, etc. The incubation time periods can be the same length of time or they can be of different lengths of time. In one embodiment, a sample is incubated for at least a first incubation time period and a second incubation time period.

Certain embodiments encompass the preparation of a biological sample for measurement of protein epitopes in order to preserve intracellular protein epitopes for subsequent detection. Such embodiments encompass a preservation step that includes contacting said sample with a preservative in an amount to achieve a final concentration sufficient to crosslink proteins, lipids, and nucleic acid molecules; a detergent step that encompasses addition of a detergent to the biological sample in an amount to achieve a final concentration sufficient to lyse any red blood cells present in the sample and permeabilize the white blood cells; and a labeling step, wherein the sample is contacted with a detectable binding agent specific for a one or more epitopes. Specific methods are described in co-pending U.S. application Ser. No. 10/928,570, which is herein incorporated by reference in its entirety. To the extent that the sample does not contain red blood cells, i.e., the sample has been previously fractionated, it is understood that the lysis step is unnecessary.

In one embodiment, the methods herein encompass a preservation step that includes contacting the sample with a preservative in an amount to achieve a final concentration sufficient to crosslink proteins, lipids and nucleic acid molecules. The preservative concentration can be between about 0.1% and about 20%, between about 0.5% and about 15%; between about 1% and about 10%, between about 1% and about 8%, between about 1% and about 4%, between about 1% and about 2%. The preservative can be added either in concentrated solution or in diluted form to achieve the desired concentration. The preservative can be any appropriate agent desired by the user, for example, aldehyde, formaldehyde, or paraformaldehyde, or formalin.

Embodiments of the methods herein further encompass a detergent step, wherein detergent is added in an amount to achieve a final concentration sufficient to lyse any present red blood cells and permeabilize white blood cells. The detergent concentration can be selected by the user based on a variety of conditions and can be in a range of between about 0.1% and about 10%; between about 0.1% and about 8%; between about 0.1% and about 7%; between about 0.1% and about 6%; between about 0.1% and about 5%; between about 0.1% and about 4%; between about 0.1% and about 3%; between about 0.1% and about 2%; between about 0.1% and about 1%.

The detergent can be selected based on a variety of factors and can be an ionic or a non-ionic detergent. Detergents are preferably selected from among non-ionic detergents. One currently preferred detergent is ethyoxylated octylphenol, which is referred to by the commercial name of TRITON® X-100 (polyoxyethylene octyl phenyl ether). In preferred embodiments, the methods are practiced with TRITON® X-100. Suitable detergents for the invention methods can permeabilize cells and retain surface epitope integrity. Ionic detergent useful in the invention further include, IGEPAL® CA-630 (octylphenoxypolyethoxyethanol), Nonidet P-40 (NP-40) (octylphenoxypolyethoxyethanol), BRIJ®-58 (polyoxyethyleneglycol dodecyl ether), and linear alcohol alkoxylates, such as PLURAFAC ® A-38 (2-methyloxirane) and PLURAFAC® A-39.

In complex cell populations such as, for example, bone marrow aspirate, undiluted peripheral blood, and peritoneal fluid, it can be useful to distinguish cell subsets by surface markers and detect intracellular phospho-epitope staining in one procedure. Embodiments of the methods provided by the present invention encompass measurements of protein epitopes that preserves intracellular protein epitopes for subsequent detection and that are amenable to be used for combining intracellular epitope detection with detection of cell surface epitopes. In methods provided by the invention, both intracellular and extracellular epitopes can remain intact so as to allow subsequent measurement by cytometric analysis. For example, the surface detection of typical bone marrow blast markers including, for example, CD34 can be combined with intracellular epitope detection.

In a further embodiment, the methods encompass a further alcohol step that encompasses contacting the biological sample with alcohol in an amount to achieve a final concentration sufficient to unmask cellular epitopes that are lost due to cross-linking during the fixation step. As described herein, the alcohol step can preserve the majority of extracellular epitopes and can be adjusted by the user in length of incubation, temperature and concentration depending on the epitopes to be preserved.

A final alcohol concentration based on other variables including, for example, incubation time, temperature and particular epitopes targeted for unmasking and measurement can be readily selected. The final alcohol concentration can be between about 25% and about 90%, between about 30% and about 80%, between about 35% and about 65%, between about 40% and about 60%, between about 45% and about 55%. The alcohol can further be selected from the group consisting of ethanol and methanol. If desired, acetone can be substituted for alcohol in the alcohol step. The sample can be contacted with alcohol or acetone at a temperature, for example, about −30° C., about −20° C., about −10° C., about '15° C., about 0° C., about 4° C., about 6° C., about 8° C., or any other temperature that facilitates the unmasking of intracellular epitopes without reducing the reactivity of cell surface epitopes.

In one embodiment of the invention, a phosphoprotein of at least one signal transduction pathway is activated to propagate a signal. The activation level of the phosphoprotein is generally determined using capture molecules. As used herein, the term “capture molecule” refers to any molecule or complex of molecules capable of binding to a protein under suitable conditions. Thus, a capture molecule includes any molecule, e.g., protein, small organic molecule, carbohydrates (including polysaccharides), polynucleotide, lipids, etc. The selection of those conditions is well known, as well as techniques to vary or modify the binding conditions. For example, it is well known that temperature, pH and time of incubation all play a role in binding. Generally, the binding occurs with sufficient specificity to exclude significant binding to more than one ligand. In certain embodiments, the binding of the capture molecule is specific for the activated form of the phosphoprotein and thus the capture molecule does not significantly bind to the non-activated form of the phosphoprotein. In some embodiments, the capture molecule is an antibody or ligand binding fragment or analog thereof. The capture molecule can also be other proteins or nucleic acids, or portions or analogs thereof, which bind signal transduction pathway phosphoprotein in the practice of certain embodiments of the invention.

In preferred embodiments, a capture molecule is an antibody, especially monoclonal antibodies. The term antibody as used herein refers to immunoglobulin molecules and immunologically active portions of immunoglobulin (Ig) molecules. Such antibodies include, but are not limited to, polyclonal, monoclonal, mono-specific polyclonal antibodies, antibody mimics, chimeric, single chain, Fab, Fab′ and F(ab′)₂ fragments, Fv, and an Fab expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain can be a kappa chain or a lambda chain. Reference herein to antibodies includes a reference to all such classes, subclasses and types of antibody species.

It has been shown that fragments of an antibody can perform the function of binding antigens. As used herein “antigen binding fragments” includes, but is not limited to: (i) the Fab fragment consisting of V_{L}, V_{H}, C_{L} and C_{H1} domains; (ii) the Fd fragment consisting of the V_{H} and C_{H1} domains; (iii) the Fv fragment consisting of the V_{L} and V_{H} domains of a single antibody; (iv) the dAb fragment which consists of a V_{H} domain; (v) isolated CDR regions; (vi) F(ab′)₂ fragments (vii) single chain Fv molecules (scFv), wherein a V_{H} domain and a V_{L} domain are linked by a peptide linker which allows the two domains to associate to form an antigen binding site (Bird et al., Science 242:423-426 (1988); Huston et al., Proc. Natl. Acad. Sci. USA 85:5879-5883 (1988)); (viii) bispecific single chain Fv dimers (PCT/US92/09965) and (ix) diabodies, multivalent or multispecific fragments constructed by gene fusion (WO94/13804; P. Holliger et al., Proc. Natl. Acad. Sci. USA 90:6444-6448 (1993)).

The capture molecules of the invention can comprise a fluorescent label. By fluorescent label is meant a molecule that can be directly (i.e., a primary label) or indirectly (i.e., a secondary label) detected; for example a label can be visualized and/or measured or otherwise identified so that its presence or absence can be known. A compound can be directly or indirectly conjugated to a label which provides a detectable signal, e.g. fluorescers, enzyme, antibodies, particles such as magnetic particles, chemiluminescers, or specific binding molecules, etc. Specific binding molecules include pairs, such as biotin and streptavidin, digoxin and antidigoxin, etc. Labels include, but are not limited to, fluorescent labels and enzymes. In general, labels can be colored or luminescent dyes or moieties; and binding partners. Labels can also include enzymes (horseradish peroxidase, etc.) and magnetic particles. In a certain embodiment, the detection label is a primary label. A primary label is one that can be directly detected, such as a fluorophore. In certain embodiments, the labels include chromophores or phosphors but are preferably fluorescent dyes or moieties. Fluorophores can be either “small molecule” fluores, or proteinaceous fluores.

By fluorescent label is meant any molecule that can be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, CASCADE BLUE™, Texas Red, IAEDANS, EDANS, BODIPY FL, LC Red 640, Cy 5, Cy 5.5, LC Red 705 and Oregon green. Suitable optical dyes are described in the 1996 Molecular Probes Handbook by Richard P. Haugland, herein incorporated by reference. Suitable fluorescent labels also include, but are not limited to, green fluorescent protein (GFP; Chalfie et al., Science 263(5148):802-805 (1994); and EGFP; Clontech—Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki et al., J. Immunol. 150(12):5408-5417 (1993)), O-galactosidase (Nolan et al., Proc Natl Acad Sci USA 85(8):2603-2607 (1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558). All of the above-cited references are incorporated herein by reference.

Additional labels for use in the present invention include: ALEXA®-Fluor dyes (ALEXA® Fluor 350, ALEXA® Fluor 430, ALEXA® Fluor 488, ALEXA® Fluor 546, ALEXA® Fluor 568, ALEXA® Fluor 594, ALEXA® Fluor 633, ALEXA® Fluor 660, ALEXA® Fluor 680), CASCADE BLUE™, CASCADE YELLOW™ and R-phycoerythrin (PE)(Molecular Probes)(Eugene, Oreg.), FITC, Rhodamine, and Texas Red (Pierce, Rockford, Ill.), Cy5, Cy5.5, Cy7 (Amersham Life Science, Pittsburgh, Pa.). Tandem conjugate protocols for Cy5PE, Cy5.5PE, Cy7PE, Cy5.5APC, Cy7APC are known in the art. Quantitation of fluorescent probe conjugation can be assessed to determine degree of labeling and protocols including dye spectral properties are known in the art. In another embodiment, the fluorescent label is a GFP and, in at least some embodiments, a renilla, ptilosarcus, or aequorea species of GFP.

In a certain embodiment, a secondary detectable label is used. A secondary label is one that is indirectly detected; for example, a secondary label can bind or react with a primary label for detection, can act on an additional product to generate a primary label (e.g. enzymes), etc. Secondary labels include, but are not limited to, one of a binding partner pair; chemically modifiable moieties; nuclease inhibitors, enzymes such as horseradish peroxidase, alkaline phosphatases, luciferases, etc.

In a certain embodiment, the secondary label is a binding partner pair. For example, the label can be a hapten or antigen, which will bind its binding partner. For example, suitable binding partner pairs include, but are not limited to: antigens (such as proteins (including peptides) and small molecules) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid--nucleic acid binding proteins pairs are also useful. Suitable binding partner pairs include, but are not limited to, biotin (or imino-biotin) and streptavidin, digeoxinin and Abs, and PROLINX™ reagents.

In a certain embodiment, the binding partner pair comprises an antigen and an antibody that will specifically bind to the antigen. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴-10⁻⁶ M⁻¹, with less than about 10⁻⁵ to 10⁻⁹ M⁻¹ being preferred and less than about 10⁻⁷-10⁻⁹ M⁻¹ being particularly preferred.

In a certain embodiment, the secondary label is a chemically modifiable moiety. In this embodiment, labels comprising reactive functional groups are incorporated into the molecule to be labeled. The functional group can then be subsequently labeled (e.g. either before or after the assay) with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo- or hetero-bifunctional linkers as are well known (see 1994 Pierce Chemical Company catalog, technical section on cross-linkers, pages 155-200, incorporated herein by reference).

In certain embodiments, multiple fluorescent labels are employed with the capture molecules of the present invention. In a preferred embodiment, at least two fluorescent labels are used which are members of a fluorescence resonance energy transfer (FRET) pair. FRET is a phenomenon known in the art wherein excitation of one fluorescent dye is transferred to another without emission of a photon. A FRET pair consists of a donor fluorophore and an acceptor fluorophore. The fluorescence emission spectrum of the donor and the fluorescence absorption spectrum of the acceptor must overlap, and the two molecules must be in close proximity. The distance between donor and acceptor at which 50% of donors are deactivated (transfer energy to the acceptor) is defined by the Forster radius (Ro), which is typically 10-100 Å. Changes in the fluorescence emission spectrum comprising FRET pairs can be detected, indicating changes in the number of pairs that are in close proximity (i.e., within 100 Å of each other). This will typically result from the binding or dissociation of two molecules, one of which is labeled with a FRET donor and the other of which is labeled with a FRET acceptor, wherein such binding brings the FRET pair in close proximity. Binding of such molecules will result in an increased fluorescence emission of the acceptor and/or quenching of the fluorescence emission of the donor. FRET pairs (donor/acceptor) useful in the invention include, but are not limited to, EDANS/fluorescien, IAEDANS/fluorescein, fluorescein/tetramethylrhodamine, fluorescein/LC Red 640, fluorescein/Cy 5, fluorescein/Cy 5.5, and fluorescein/LC Red 705.

In another aspect of FRET, a fluorescent donor molecule and a nonfluorescent acceptor molecule (“quencher”) can be employed. In this application, fluorescent emission of the donor will increase when quencher is displaced from close proximity to the donor and fluorescent emission will decrease when the quencher is brought into close proximity to the donor. Useful quenchers include, but are not limited to, TAMRA, DABCYL, QSY 7, and QSY 33. Useful fluorescent donor/quencher pairs include, but are not limited to EDANS/DABCYL, Texas Red/DABCYL, BODIPY/DABCYL, Lucifer yellow/DABCYL, coumarin/DABCYL, and fluorescein/QSY 7 dye.

FRET and fluorescence quenching of stained cells allow for monitoring of binding of labeled molecules over time, providing continuous information regarding the time course of binding reactions. Changes in the degree of FRET can be determined as a function of the change in the ratio of the amount of fluorescence from the donor and acceptor moieties, a process referred to as “ratioing.” Changes in the absolute amount of substrate, excitation intensity, and turbidity or other background absorbance in the sample at the excitation wavelength affect the intensities of fluorescence from both the donor and acceptor approximately in parallel. Therefore, the ratio of the two emission intensities is a more robust and preferred measure of cleavage than either intensity alone.

The ratiometric fluorescent reporter system described herein has significant advantages over existing reporters for protein integration analysis, as it allows sensitive detection and isolation of both expressing and non-expressing single living cells. In a certain embodiment, the assay system uses a non-toxic, non-polar fluorescent substrate which is easily loaded and then trapped intracellularly. Modification of the fluorescent substrate by a cognate protein yields a fluorescent emission shift as substrate is converted to product. Because the reporter readout is ratiometric it is unique among reporter protein assays in that it controls for variables such as the amount of substrate loaded into individual cells. The stable, easily detected, intracellular readout eliminates the need for establishing clonal cell lines prior to expression analysis. This system and other analogous flow sorting systems can be used to isolate cells having a particular receptor clustering and/or activation profile from pools of millions of viable cells.

Antibody conjugation can be performed using standard procedures (drmr.com.abcon) or by using protein-protein/protein-dye crosslinking kits from Molecular Probes (Eugene, Oreg.). Conjugation of the label moiety to the detection molecule, such as for example an antibody, is a standard manipulative procedure in immunoassay techniques. See, for example, O'Sullivan et al., 1981, “Methods for the Preparation of Enzyme-antibody Conjugates for Use in Enzyme Immunoassay,” in Methods in Enzymology, Langone and Van Vunakis, Eds., Vol. 73 (Academic Press, New York, N.Y.), pp. 147-166. Conventional methods are available to bind the label moiety covalently to proteins or polypeptides. For example, coupling agents such as dialdehydes, carbodiimides, dimaleimides, bis-imidates, bis-diazotized benzidine, and the like, can be used to label antibodies with the above-described fluorescent, chemiluminescent, and enzyme labels. See, for example, U.S. Pat. No. 3,940,475 (fluorimetry) and U.S. Pat. No. 3,645,090 (enzymes); Hunter et al., Nature, 144:945 (1962); David et al., Biochemistry, 13:1014-1021 (1974); Pain et al., J. Immunol Methods, 40:219-230 (1981); and Nygren J., Histochem. and Cytochem., 30:407-412 (1982). Fluorescent or chemiluminescent labels can be used to increase amplification and sensitivity to about 5-10 pg/ml, or better.

In the embodiments of the invention the capture molecules are activation specific. The methods and compositions of the present invention can be used to detect any particular phosphoprotein isoform in a sample that is antigenically detectable and antigenically distinguishable from other phosphoprotein isoforms which are present in the sample. For example, as demonstrated (see, e.g., the Examples) and described herein, the activated phosphoprotein capture molecules of the present invention can be used in the present methods to identify distinct signaling cascades of a subset or subpopulation of complex cell populations; and the ordering of phosphoprotein activation (e.g., kinase activation) in potential signaling hierarchies. Further, in the methods of the present invention, the use of flow cytometry, particularly polychromatic flow cytometry, permits the multi-dimensional analysis and functional assessment of the signaling pathway in single cells.

As used herein, the terms “activated phosphoprotein capture molecule” refer to a capture molecule (i.e., an antibody) that specifically binds to a corresponding and specific antigen of an activated isoform of a phosphoprotein. In certain embodiments, the corresponding and specific antigen is a specific isoform of a phosphoprotein. In another embodiment, the binding of the activated phosphoprotein capture molecule is indicative of a specific activation state of a phosphoprotein. In other embodiments, the binding of an activated phosphoprotein capture molecule to a corresponding isoform of an activated phosphoprotein is indicative of the identity of the activated phosphoprotein and of the activation state of the activated phosphoprotein. In certain embodiments, the binding of the capture molecule is specific for the activated isoform of the phosphoprotein and thus the capture molecule does not significantly bind to one or more “non-activated” isoforms of the phosphoprotein.

In an embodiment, the activated phosphoprotein capture molecule is a peptide comprising a recognition structure that binds to a target structure on an activated phosphoprotein. A variety of recognition structures are well known in the art and can be made using methods known in the art, including by phage display libraries (see e.g., Gururaja et al., Chem. Biol. (2000) 7:515-27; Houimel et al., Eur. J. Immunol. (2001) 31:3535-45; Cochran et al., J. Am. Chem. Soc. (2001) 123:625-32; Houimel et al., Int. J. Cancer (2001) 92:748-55, each incorporated herein by reference). In a certain embodiment, the activated phosphoprotein capture molecule comprises the following recognition structure: SKVILFE—random peptide loop—SKVILFE. Capture molecules having such recognition structures can bind with high affinity to specific target structures. Further, fluorophores can be attached to such capture molecules for use in the methods of the present invention.

A variety of recognitions structures are known in the art (e.g., Cochran et al., J. Am. Chem. Soc. (2001) 123:625-32; Boer et al., Blood (2002) 100:467-73, each expressly incorporated herein by reference) and can be produced using methods known in the art (see e.g., Boer et al., Blood (2002) 100:467-73; Gualillo et al., Mol. Cell. Endocrinol. (2002) 190:83-9, each expressly incorporated herein by reference), including for example combinatorial chemistry methods for producing recognition structures such as polymers with affinity for a target structure on an activatable protein (see e.g., Barn et al., J. Comb. Chem. (2001) 3:534-41; Ju et al., Biotechnol. (1999) 64:232-9, each expressly incorporated herein by reference). In another embodiment, the activated phosphoprotein capture molecule is one that only binds to an isoform of a specific phosphoprotein that is phosphorylated and does not bind to the isoform of this phosphoprotein when it is not phosphorylated or is nonphosphorylated. In another embodiment, the activated phosphoprotein capture molecule is a protein that only binds to an isoform of a phosphoprotein that is intracellular and not extracellular, or vice versa.

Antibodies, many of which are commercially available have been produced which specifically bind to the phosphorylated isoform of a phosphoprotein but do not specifically bind to a non-phosphorylated isoform of a phosphoprotein. Particularly, many such antibodies have been produced which specifically bind to phosphorylated, activated isoforms of protein kinases and are sometimes referred to herein as kinase activation state antibodies or grammatical equivalents thereof. In certain embodiments, antibodies for use in the present invention include: antibodies against phospho-p44/42 MAP kinase (Thr202/Tyr204), phospho-TYK2 (Tyr1054/1055), phospho-p38 MAP kinase (Thr180/Tyr182), phospho-PKC-PAN substrate, phospho-PKA-substrate, phospho-SAPK/JNK (Thr183/Tyr185), phospho-tyrosine (P-tyr-100), p44/42 MAPK, phospho-MEK1/2 (Ser217/221), phospho-p90RSK (Ser381), p38 MAPK, JNK/SAPK, phospho-Raf1 (Ser259), phosphoElk-1 (Ser383), phospho-CREB (Ser133), phosphoSEK1/MKK4 (Thr261), phospho-Jun (Ser 63), phosphoMKK3/MKK6 (Ser189/207), AKT, phospho FKHR, FKHR, phospho-Gsk3 alp21, pAFX, PARP, BAD, BADser 112, BADser 136, phospho-BADser 155, p27, p21, cFLIP, MYC, p53, NFKB, Ikkα, Ikkβ, phospho-tyrosine and phospho-threonine combination. In certain embodiments, these antibodies are monoclonal antibodies. In certain embodiments these antibodies are used in various combinations.

Control capture molecules can also be used in the present invention. In some embodiments, the control capture molecule binds to an epitope a protein present in an activatable cell that is unaffected by the signaling transduction pathway activation. In one embodiment, the control capture molecule binds to a cell surface receptor that identifies a certain cell subtype within a sample containing a mixed population of cell types. In a certain embodiment, control capture molecules bind to epitopes in both activated and non-activated forms of a phosphoprotein of at least one signal transduction pathway. Such control capture molecules can be used to determine the amount of non-activated plus activated signal transduction pathway phosphoprotein in a sample. In another embodiment, control capture molecules bind to epitopes present in non-activated isoforms of a phosphoprotein but absent in activated isoforms of a phosphoprotein. Such control capture molecules can be used to determine the amount of non-activated phosphoprotein in a sample. Both types of control capture molecules can be used to determine if a change in the activation state of a phosphoprotein, for example from samples before and after treatment with a candidate bioactive agent coincides with changes in the amount of non-activated phosphoprotein. For example, such control capture molecules can be used to determine whether an increase in activated phosphoprotein of at least one signal transduction pathway is due to activation of a phosphoprotein, or due to increased expression of the phosphoprotein, or both. The use of control capture molecules is further exemplified in co-pending U.S. application Ser. No. 11/276,948, which is herein incorporated by reference in its entirety. Preferably, the control capture molecule binds to the same cell that the activated phosphoprotein capture molecule binds, albeit at an epitope that is unactivated by the pan-kinase inhibitor.

In most embodiments, a control capture molecule is added to the “same tube” as the activated phosphoprotein capture molecules. By same tube, it is understood that it is the same reaction container, be that a container, a tube, or a well in a microtiter plate or the like. The control capture molecule thus differs from the traditional isotype controls in that it provides a truer valuation of the base line fluorescence of the test cell. For example, in those embodiments where the cell being evaluated is a hematopoietic progenitor cell, the control capture molecules can bind to CD34 and CD117, which are activation independent markers of cells containing the receptor for SCF. This will permit the identification of such cells and the setting of the baseline fluorescence of the CD34⁺, CD117⁺ cell population. In other possible embodiments other cell surface markers, including other receptors that are found on leukemic stem cell populations (for example, CD135/FLT-3 receptor, PDGFR, IL-3R), and additional cell surface markers that can be used to monitor leukemic cell differentiation, including but not limited to CD13, CD15, CD16, CD33, CD64, can be used identify cell populations in a sample. When those cells are then activated and fluorescence shifts, the degree of shift is accordingly a truer measure of the increase in fluorescence. The use of these controls therefore provides a better way to reduce the background fluorescence of the cells being evaluated. Since phosphorylation states of phosphoproteins have been traditionally difficult to identify, let alone quantitate, controlling the background is important to the overall sensitivity of the methods of the present invention.

Assay systems utilizing a capture molecule and a fluorescent label to quantify captured molecules are well known. Examples of immunoassays useful in the invention include, but are not limited to, fluoroluminescence assay (FLA), chemiluminescence assay (CA), enzyme-linked immunosorbant assay (ELISA) and the like. See, for example, Johnstone and Thorpe, 1996, In: Blackwell, Immunochemistry in Practice, 3rd ed. (Blackwell Publishing, Malden, Mass.); Ausbul et al., eds., 2003, Current Protocols in Molecular Biology, Wiley & Sons (Hoboken, N.J.); Ghindilis et al., eds., 2003, Immunoassay Methods and Protocols, (Blackwell Publishing, Malden, Mass.); and U.S. Patent Publication No. 20030044865. The immunoassay can be a solid phase assay, a liquid phase assay, and the like.

The immunoassay, in one embodiment, can be designed for an automated, high-throughput instrument. For example, the ACCESS® family of instruments by Beckman Coulter, Inc. is well suited to effectuate the methods of the invention. The ACCESS® Immunoassay System allows for rapid throughput of up to 100 tests per hour through the use of a reaction vessel loader that has the capacity for up to 3 hours of continuous sample processing.

In an embodiment, flow cytometry is used to detect fluorescence. Other methods of detecting fluorescence can also be used, e.g., Quantum dot methods (see, e.g., Goldman et al., J. Am. Chem. Soc. (2002) 124:6378-82; Pathak et al., J. Am. Chem. Soc. (2001) 123:4103-4; and Remade et al., Proc. Natl. Sci. USA (2000) 18:553-8, each incorporated herein by reference).

For a solid-phase immunoassay, the capture molecule is immobilized to a solid support. Immobilization conventionally is accomplished by insolubilizing the capture molecule either before the assay procedure, as by adsorption to a water-insoluble matrix or surface (U.S. Pat. No. 3,720,760) or as by non-covalent or covalent coupling (for example, using glutaraldehyde or carbodiimide cross-linking, with or without prior activation of the support with, e.g., nitric acid and a reducing agent as described in U.S. Pat. No. 3,645,852 or in Rotmans et al.; J. Immunol. Methods, 57:87-98 (1983)), or afterward, e.g., by immunoprecipitation.

The solid phase used for immobilization can be any inert support or carrier that is essentially water insoluble and useful in immunometric assays, including supports in the form of, e.g., surfaces, particles, porous matrices, etc. Examples of commonly used supports include small sheets, SEPHADEX® gels, polyvinyl chloride, plastic beads, and assay plates or test tubes manufactured from polyethylene, polypropylene, polystyrene, and the like, including 96-well microtiter plates, as well as particulate materials such as filter paper, agarose, cross-linked dextran, and other polysaccharides. Capture molecules can also be immobilized on a substrate, such as a polymeric bead, colloidal metal or iron oxide particle. Beads can be plastic, glass, or any other suitable material, typically in the 1-20 micron range. In some embodiments, paramagnetic beads are used. Colloidal metal particles such as colloidal gold and silver particles and iron oxide particles can be prepared using many different procedures commercially available or otherwise known to those skilled in the art.

Alternatively, reactive water-insoluble matrices such as cyanogen-bromide-activated carbohydrates and the reactive substrates described in U.S. Pat. Nos. 3,969,287; 3,691,016; 4,195,128; 4,247,642; 4,229,537; and 4,330,440 can be used for capture molecule immobilization. In one embodiment, the immobilized capture molecules are coated on a microtiter plate, and in another embodiment the solid phase is a multi-well microtiter plate that can analyze several samples at one time.

The solid phase is coated with the capture molecules as defined above, which can be linked by a non-covalent or covalent interaction or physical linkage as desired. Techniques for attachment include those described in U.S. Pat. No. 4,376,110 and the references cited therein. If covalent, the plate or other solid phase is incubated with a cross-linking agent together with the capture molecules under conditions well known in the literature.

Commonly used cross-linking agents for attaching the capture molecules to the solid-phase substrate include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-((p-azidophenyl)-dithio)propioimidate yield photoactivatable intermediates capable of forming cross-links in the presence of light.

The coated plates are then typically treated with a blocking agent that binds non-specifically with and saturates the unoccupied binding sites to prevent unwanted binding of the free ligand to the excess sites on the wells of the plate. Examples of appropriate blocking agents for this purpose include, e.g., gelatin, bovine serum albumin, egg albumin, casein, and non-fat milk. The blocking treatment typically takes place under conditions of ambient temperatures for about 1-4 hours, typically about 1.5 to 3 hours.

The amount of capture molecule employed is sufficiently large to give a good signal in comparison with the calibration standards, but is generally not in molar excess compared to the maximum expected level of a phosphoprotein of at least one signal transduction pathway that is of interest in the sample. For sufficient sensitivity, the amount of test sample should be added such that the immobilized capture molecules are in molar excess of the maximum molar concentration of free analyte of interest anticipated in the test sample after appropriate dilution of the sample.

Generally, the conditions for incubation of sample and immobilized capture molecule are selected to maximize analytical sensitivity of the assay to minimize dissociation, and to ensure that sufficient analyte of interest that is present in the sample binds with the immobilized capture molecule. It is understood that the selection of optimum reaction conditions generally requires only routine experimentation. The incubation is accomplished at fairly constant temperatures, ranging from about 0° C. to about 40° C., generally at or about room temperature. The time for incubation is generally no greater than about 10 hours. The duration of incubation can be longer if a protease inhibitor is added to prevent proteases in the test sample from degrading the phosphoprotein of at least one signal transduction pathway of interest.

The present specification provides methods for determining a phosphoprotein activation profile in a sample containing hematopoietic cells. A phosphoprotein activation profile refers to degree to which a phosphoprotein is phosphorylated. Such a profile can be assessed from a single timepoint, or can be measured from two or more timepoints. Additionally, such a profile can be assessed under a single condition or under a plurality of conditions. In one embodiment, a phosphoprotein activation profile can comprise a qualitative measurement of whether a phosphoprotein is phosphorylated or unphosphorylated. In another embodiment, a phosphoprotein activation profile can comprise a quantitative measurement of the degree to which a phosphoprotein is phosphorylated. In other embodiments, a phosphoprotein activation profile determined from two or more timepoints can be used to calculate the phosphorylation rate of a phosphoprotein and assess how various conditions can affect that rate.

Typically, a phosphoprotein activation profile for the same phosphoprotein can be determined under two or more conditions and the resulting profiles compared. For example, measuring the phosphorylation of a phosphoprotein under conditions where the cells are treated or untreated with a phosphoprotein activator. As another example, measuring the phosphorylation of a phosphoprotein under conditions where the cells are treated or untreated with a phosphoprotein inhibitor. As yet another example, measuring the phosphorylation of a phosphoprotein under conditions where the cells were obtained from a healthy individual or an individual having or suspected of having a leukemia.

In certain embodiments, methods for determining a phosphoprotein activation profile in a sample containing hematopoietic cells comprises contacting a preserved activated sample with a plurality of fluorescently labeled capture molecules, said plurality of capture molecules comprising at least one capture molecule capable of binding to an activated phosphoprotein in the sample and at least one control capture molecule, wherein the control capture molecule binds to a protein in the hematopoietic cells that is unactivated by the phosphoprotein activator. In such an embodiment, the preserved, activated hematopoietic cells captured by the capture molecules are detected using one of the immunoassay formats described above. In certain embodiments, the fluorescence detection detects the labeled capture molecules bound to the activated state of the unmasked intracellular epitopes. The preserved hematopoietic cells are similarly detected. Therefore in certain embodiments, the immunoassay detects fluorescence of the preserved cells captured by the binding of the control capture molecules.

When using fluorescent labeled components in the methods and compositions of the present invention, it will recognized that different types of fluorescent monitoring systems, e.g., flow cytometry systems, can be used to practice the invention. In some embodiments, flow cytometry systems are used or systems dedicated to high throughput screening, e.g. 96-well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation which has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation.

In another embodiment, capture molecules are immobilized using beads analogous to those known and used for standardization in flow cytometry. Attachment of a multiplicity of activation state specific capture molecules to beads can be done by methods known in the art and/or described herein. Such conjugated beads can be contacted with a sample, preferably a cell extract, under conditions which allow for a multiplicity of activated rphosphoproteins, if present, to bind to the multiplicity of immobilized capture molecules. A second multiplicity of capture molecules comprising control capture molecules capable of binding to a phosphoprotein in its non-activation state which are uniquely labeled can be added to the immobilized activated phosphoprotein capture molecule complex and the beads can be sorted by flow cytometry on the basis of the presence of each label, wherein the presence of label indicates binding of corresponding second capture molecule and the presence of corresponding activated phosphoprotein.

Once the fluorescences of the activated phosphoprotein capture molecules and control capture molecules have been detected, their fluorescence can be compared. The terms “correlate” or “compare”, or their grammatical equivalents, are intended to be used interchangeably, unless the particular context connotes a different meaning. Correlating/comparing is meant comparing, in any way, the performance and/or results of a first analysis with the performance and/or results of a second analysis. For example, as described in greater detail below, the level of SCF-stimulated pERK activation in a bone marrow sample from an AML patient differs from the level of SCF-stimulated pERK activation in a bone marrow sample from a normal individual. By identifying a particular activity level, the phosphoprotein's activity can act as an indicator or as a predictor of prognosis for a specific disease condition, thereby “correlating” activity with disease condition status. In the diseases and conditions discussed elsewhere herein, the disease or condition can be characterized by an increased activation response when exposed to the phosphoprotein activator. Similarly, as described elsewhere herein the responsiveness of the disease or condition to treatment can be identified by evaluating the fluorescence of the various capture molecules to identify a decrease in the phosphoprotein activation response.

In other embodiments, the fluorescence of the activated phosphoprotein of at least one signal transduction pathway can be evaluated against the fluorescence of an unactivated phosphoprotein. The purpose in these evaluations is to discern whether a difference exists between the fluorescence signal generated by the activated vs. unactivated or the activated vs. standardized reference sample. Generally, these evaluations constitute a second correlation step. In at least some embodiments, the unactivated reference sample is a second aliquot of the sample.

The standardized reference sample is, in one embodiment, a manufacturer-set value of expected fluorescence of an activated or an unactivated cell sample treated under highly reproducible conditions. These types of standardized reference values are intended to serve as surrogates to the values that the end user would achieve were they to run a parallel sample. A purpose of these standardized reference values is to achieve efficiency in labor for the end user in that the end user would not need to run a parallel sample, and the labor and reagent costs associated with preparing and running such a parallel sample. Manufacturers of diagnostic reagent kits, such as Beckman Coulter, are well accustomed to preparing standardized reference values for their reagents and kits.

The immunoassay of the present invention provides a higher degree of specificity than the present assays described in the art. Specificity is provided through the use of vigorous controls. Since the strength of many signals is low, as well as transient in nature, the high background levels do not allow for application of the assays in a clinical setting. In one embodiment of the present invention, CD34⁺, CD117⁺ cells are identified by flow cytometry using side and forward scatter coupled with expression of the cell surface markers. In a further embodiment, the phosphorylation of activated phosphoprotein is measured in stained CD34⁺, CD117⁺ cells treated with a cytokine for various amounts of time. This phosphorylation can be reported as a mean fluorescence intensity of stimulated cells over the baseline level at various time periods of stimulation, as a frequency of positive stained cells, or as the fold change of the positive/negative ratio at various time periods of stimulation over the baseline level. Embodiments of the methods herein provide meaningful data measuring the response of single cells in a total cell population, particularly where the target population of cells exists as a low percentage of the total cell population. For hematopoietic diseases, characterizing the kinetics of baseline phosphoprotein activation profiles in normal, healthy tissue is essential, in order to fully understand both the major differences as well as the fine distinctions observed in the diseased state.

In various additional embodiments of the methods herein, activated phosphoprotein kinetic profiles are measured from a healthy individual as well as an individual suspected of having a hematopoetic disease or condition. The resulting profiles are compared to one another in order to confirm whether or not the individual suspected of having a hematopoetic disease or condition can be diagnosed as such. In an embodiment, activated phosphoprotein kinetic profiles of SCF-, FL-, IL-3-, and GM-CSF-mediated phosphorylation of S6, ERK, STAT3, STAT5, or any combination thereof in CD34⁺, CD117⁺ cells are measured. In another embodiment, the hematopoetic disease or condition is a leukemia, such as, e.g., an AML, an ALL, a CLL, a lymphoma, a follicular lymphoma, or a multiple myeloma.

In an embodiment, after SCF activation of a sample, increased phosphorylation of ERK and AKT in the sample from a healthy individual, and only an increased phosphorylation of AKT, but not ERK in the sample from an individual suspected of having a hematopoetic disease or condition is indicative of the disease or condition.

In another embodiment, increased basal levels of phosphorylated STAT5 in a sample from an individual suspected of having a hematopoetic disease or condition as compared to the basal levels of phosphorylated STAT5 in a sample from a healthy individual, is indicative of the disease or condition. In aspects of this embodiment, increased basal levels of phosphorylated STAT5 in a sample from an individual suspected of having a hematopoetic disease or condition is, e.g., 25% or more higher, 50% or more higher, 75% or more higher, 100% or more higher, 200% or more higher, or 300% or more higher, than the basal levels of phosphorylated STAT5 measured in a sample from a healthy individual. In other aspects of this embodiment, increased basal levels of phosphorylated STAT5 in a sample from an individual suspected of having a hematopoetic disease or condition is between, e.g., 25% to 50% higher, 25% to 75% higher, 25% to 100% higher, 25% to 200% higher, 25% to 300% higher, 50% to 75% higher, 50% to 100% higher, 50% to 200% higher, 50% to 300% higher, 75% to 100% higher, 75% to 200% higher, 75% to 300% higher, 100% to 200% higher, or 100% to 300% higher, than the basal levels of phosphorylated STAT5 measured in a sample from a healthy individual.

In another embodiment, increased basal levels of phosphorylated S6 in a sample from an individual suspected of having a hematopoetic disease or condition as compared to the basal levels of phosphorylated S6 in a sample from a healthy individual, is indicative of the disease or condition. In aspects of this embodiment, increased basal levels of phosphorylated S6 in a sample from an individual suspected of having a hematopoetic disease or condition is, e.g., 25% or more higher, 50% or more higher, 75% or more higher, 100% or more higher, 200% or more higher, or 300% or more higher, than the basal levels of phosphorylated S6 measured in a sample from a healthy individual. In other aspects of this embodiment, increased basal levels of phosphorylated S6 in a sample from an individual suspected of having a hematopoetic disease or condition is between, e.g., 25% to 50% higher, 25% to 75% higher, 25% to 100% higher, 25% to 200% higher, 25% to 300% higher, 50% to 75% higher, 50% to 100% higher, 50% to 200% higher, 50% to 300% higher, 75% to 100% higher, 75% to 200% higher, 75% to 300% higher, 100% to 200% higher, or 100% to 300% higher, than the basal levels of phosphorylated S6 measured in a sample from a healthy individual.

In another embodiment, increased basal levels of phosphorylated AKT in a sample from an individual suspected of having a hematopoetic disease or condition as compared to the basal levels of phosphorylated STAT5 in a sample from a healthy individual, is indicative of the disease or condition. In aspects of this embodiment, increased basal levels of phosphorylated AKT in a sample from an individual suspected of having a hematopoetic disease or condition is, e.g., 25% or more higher, 50% or more higher, 75% or more higher, 100% or more higher, 200% or more higher, or 300% or more higher, than the basal levels of phosphorylated AKT measured in a sample from a healthy individual. In other aspects of this embodiment, increased basal levels of phosphorylated AKT in a sample from an individual suspected of having a hematopoetic disease or condition is between, e.g., 25% to 50% higher, 25% to 75% higher, 25% to 100% higher, 25% to 200% higher, 25% to 300% higher, 50% to 75% higher, 50% to 100% higher, 50% to 200% higher, 50% to 300% higher, 75% to 100% higher, 75% to 200% higher, 75% to 300% higher, 100% to 200% higher, or 100% to 300% higher, than the basal levels of phosphorylated AKT measured in a sample from a healthy individual.

In yet another embodiment, after GM-CSF activation of a sample, increased phosphorylation of AKT in the sample from an individual suspected of having a hematopoetic disease or condition, but no increased phosphorylation is AKT in the sample from a healthy individual, is indicative of the disease or condition. In aspects of this embodiment, increased phosphorylated of AKT in a sample from an individual suspected of having a hematopoetic disease or condition is, e.g., 25% or more higher, 50% or more higher, 75% or more higher, 100% or more higher, 200% or more higher, or 300% or more higher, than the levels of phosphorylated AKT measured in a sample from a healthy individual. In other aspects of this embodiment, increased phosphorylated of AKT in a sample from an individual suspected of having a hematopoetic disease or condition is between, e.g., 25% to 50% higher, 25% to 75% higher, 25% to 100% higher, 25% to 200% higher, 25% to 300% higher, 50% to 75% higher, 50% to 100% higher, 50% to 200% higher, 50% to 300% higher, 75% to 100% higher, 75% to 200% higher, 75% to 300% higher, 100% to 200% higher, or 100% to 300% higher, than the levels of phosphorylated AKT measured in a sample from a healthy individual.

In still another embodiment, after SCF activation of a sample, a lower AKT phosphorylation rate in the sample from an individual suspected of having a hematopoetic disease or condition, as compared to an AKT phosphorylation rate in the sample from a healthy individual, is indicative of the disease or condition. In aspects of this embodiment, the AKT phosphorylation rate in a sample from an individual suspected of having a hematopoetic disease or condition is lower by, e.g., 25% or more, 50% or more, 75% or more, 100% or more, 125% or more, or 150% or more, then the AKT phosphorylation rate in a sample from a healthy individual. In other aspects of this embodiment, the AKT phosphorylation rate in a sample from an individual suspected of having a hematopoetic disease or condition is lower by, e.g., 25% to 50%, 25% to 75%, 25% to 100%, 25% to 125%, 25% to 150%, 50% to 75%, 50% to 100%, 50% to 125%, 50% to 150%, then the AKT phosphorylation rate in a sample from a healthy individual.

In an embodiment, after SCF activation of a sample, an increased level of ERK phosphorylation in the sample from an individual suspected of having a hematopoetic disease or condition, as compared to the level of ERK phosphorylation in the sample from a healthy individual, is indicative of the disease or condition. In aspects of this embodiment, the level of ERK phosphorylation in the sample from an individual suspected of having a hematopoetic disease or condition is increased by, e.g., 1-fold or more, 2-fold or more, 3-fold or more, 4-fold or more, 5-fold or more, 6-fold or more, 7-fold or more, 8-fold or more, 9-fold or more, or 10-fold or more, as compared to the level of ERK phosphorylation in the sample from a healthy individual. In other aspects, the level of ERK phosphorylation in the sample from an individual suspected of having a hematopoetic disease or condition is increased by, e.g., 1-fold to 2-fold, 1-fold to 5-fold, 1-fold to 10-fold, 2-fold to 5-fold, 2-fold to 7-fold, 2-fold to 10-fold, 3-fold to 6-fold, 3-fold to 7-fold, 3-fold to 10-fold, 5-fold to 7-fold, or 5-fold to 10-fold, as compared to the level of ERK phosphorylation in the sample from a healthy individual.

In another embodiment, after FL activation of a sample, an increased level of ERK phosphorylation in the sample from an individual suspected of having a hematopoetic disease or condition, as compared to the level of ERK phosphorylation in the sample from a healthy individual, is indicative of the disease or condition. In aspects of this embodiment, the level of ERK phosphorylation in the sample from an individual suspected of having a hematopoetic disease or condition is increased by, e.g., 1-fold or more, 2-fold or more, 3-fold or more, 4-fold or more, 5-fold or more, 6-fold or more, 7-fold or more, 8-fold or more, 9-fold or more, or 10-fold or more, as compared to the level of ERK phosphorylation in the sample from a healthy individual. In other aspects, the level of ERK phosphorylation in the sample from an individual suspected of having a hematopoetic disease or condition is increased by, e.g., 1-fold to 2-fold, 1-fold to 5-fold, 1-fold to 10-fold, 2-fold to 5-fold, 2-fold to 7-fold, 2-fold to 10-fold, 3-fold to 6-fold, 3-fold to 7-fold, 3-fold to 10-fold, 5-fold to 7-fold, or 5-fold to 10-fold, as compared to the level of ERK phosphorylation in the sample from a healthy individual.

In yet another embodiment, after SCF activation of a sample, an increased level of S6 phosphorylation in the sample from an individual suspected of having a hematopoetic disease or condition, as compared to the level of S6 phosphorylation in the sample from a healthy individual, is indicative of the disease or condition. In aspects of this embodiment, the level of S6 phosphorylation in the sample from an individual suspected of having a hematopoetic disease or condition is increased by, e.g., 5-fold or more, 10-fold or more, 15-fold or more, 20-fold or more, 25-fold or more, 30-fold or more, 35-fold or more, 40-fold or more, 45-fold or more, 50-fold or more, 55-fold or more, or 60-fold or more, as compared to the level of S6 phosphorylation in the sample from a healthy individual. In other aspects, the level of S6 phosphorylation in the sample from an individual suspected of having a hematopoetic disease or condition is increased by, e.g., 5-fold to 10-fold, 5-fold to 20-fold, 5-fold to 30-fold, 5-fold to 40-fold, 5-fold to 50-fold, 5-fold to 60-fold, 10-fold to 20-fold, 10-fold to 30-fold, 10-fold to 40-fold, 10-fold to 50-fold, 10-fold to 60-fold, 20-fold to 30-fold, 20-fold to 40-fold, 20-fold to 50-fold, 20-fold to 60-fold, 30-fold to 40-fold, 30-fold to 50-fold, or 30-fold to 60-fold, as compared to the level of S6 phosphorylation in the sample from a healthy individual.

In still another embodiment, after IL-3 activation of a sample, an increased level of STAT5 phosphorylation in the sample from an individual suspected of having a hematopoetic disease or condition, as compared to the level of ERK phosphorylation in the sample from a healthy individual, is indicative of the disease or condition. In aspects of this embodiment, the level of ERK phosphorylation in the sample from an individual suspected of having a hematopoetic disease or condition is increased by, e.g., 1-fold or more, 2-fold or more, 3-fold or more, 4-fold or more, 5-fold or more, or 6-fold or more, as compared to the level of STAT5 phosphorylation in the sample from a healthy individual. In other aspects, the level of STAT5 phosphorylation in the sample from an individual suspected of having a hematopoetic disease or condition is increased by, e.g., 1-fold to 2-fold, 1-fold to 3-fold, 1-fold to 4-fold, 1-fold to 5-fold, 1-fold to 6-fold, 2-fold to 3-fold, 2-fold to 4-fold, 2-fold to 5-fold, 2-fold to 6-fold, 3-fold to 4-fold, 3-fold to 5-fold, 3-fold to 6-fold, 4-fold to 5-fold, or 4-fold to 6-fold, as compared to the level of STAT5 phosphorylation in the sample from a healthy individual.

In still another embodiment, after GM-CSF activation of a sample, an increased level of STAT5 phosphorylation in the sample from an individual suspected of having a hematopoetic disease or condition, as compared to the level of ERK phosphorylation in the sample from a healthy individual, is indicative of the disease or condition. In aspects of this embodiment, the level of ERK phosphorylation in the sample from an individual suspected of having a hematopoetic disease or condition is increased by, e.g., 1-fold or more, 2-fold or more, 3-fold or more, 4-fold or more, 5-fold or more, or 6-fold or more, as compared to the level of STAT5 phosphorylation in the sample from a healthy individual. In other aspects, the level of STAT5 phosphorylation in the sample from an individual suspected of having a hematopoetic disease or condition is increased by, e.g., 1-fold to 2-fold, 1-fold to 3-fold, 1-fold to 4-fold, 1-fold to 5-fold, 1-fold to 6-fold, 2-fold to 3-fold, 2-fold to 4-fold, 2-fold to 5-fold, 2-fold to 6-fold, 3-fold to 4-fold, 3-fold to 5-fold, 3-fold to 6-fold, 4-fold to 5-fold, or 4-fold to 6-fold, as compared to the level of STAT5 phosphorylation in the sample from a healthy individual.

Embodiments herein provide the advantage of the methods herein being performed on samples quickly after the samples are obtained, with minimal processing required. The normal bone marrow samples used in embodiments herein were from healthy, adult donors. The donations were made solely for research purposes, and the donors were compensated for their time and discomfort. Once the bone marrow was aspirated, it was passed along very quickly (within an hour), with minimal processing, for experimentation. Thus, samples were fresh and relatively unperturbed.

In various embodiments of the methods herein, CD34⁺, CD117⁺ cells are used as they represent a primitive hematopoietic cell population, which includes hematopoietic stem cells (HSCs) and leukemia stem cells (LSCs). The presence of CD117 ensures that the cells of each population also possess KIT. The presence of CD34⁺ cells is important in some embodiments considering the current understanding of LSCs in AML. Following diagnosis, the majority of AMLs initially respond to treatment, but relapse, with resistant disease, frequently occurs, and is often lethal for most patients. The prevailing hypothesis suggests that current therapies reduce tumor bulk (primarily leukemic blasts without tumor induction properties) but not LSCs, which are relatively insensitive to therapy and possess the required potential to initiate relapse and resistance. Consequently, LSCs are a target of developing clinical therapies.

Various embodiments of the methods herein make use of cytokines that regulate hematopoiesis. In the examples herein, SCF, FL, IL-3, G-CSF, and GM-CSF are used because they are among the major cytokines that regulate hematopoiesis. They share a number of common properties, including autocrine/paracrine regulation, overlapping and redundant functionality, synergy in combination with other cytokines, and activation of similar signal transduction mechanisms. However, each cytokine has a distinct range of regulatory activity on the hematopoietic system, regulation that is mediated by a unique membrane-bound receptor.

In certain embodiments, the invention relates to methods of monitoring the activity of an inhibitor of one or more signal transduction pathways which have been administered to patients. When these patients are receiving a signal transduction pathway protein inhibitor, the inhibitor shuts down or reduces activation of a phosphoprotein. Normally, these inhibitors titrate from the patient or test sample over time and are re-administered to maintain their effectiveness/efficacy. At a time just prior to the re-administration of the inhibitor, a patient sample (or in the case of a tissue culture assay—a cell sample) can be obtained and the white blood cells in that sample can be tested using the assays of the present invention. If the inhibitor is being effective to treat the disease or condition, the assay will reveal a change in the activation response toward a response that is more similar to that observed in a normal, i.e., non-diseased, sample. Thus, the present invention, in at least one embodiment, provides a highly effective, sensitive assay to monitor the progression of the clinical treatment of diseases or conditions characterized by an aberrant signal transduction pathway phosphoprotein activation.

In certain embodiments, the method comprises simultaneously determining the presence of activated isoforms of a multiplicity of signal transduction pathway phosphoproteins using a multiplicity of antibodies that specifically bind to actived phosphorylated isoforms of the multiplicity of activated phosphoproteins.

Additional ways for determining phosphoprotein activation are provided by the present invention. Substrates that are specifically recognized by protein kinases and phosphorylated thereby are known. Antibodies that specifically bind to such phosphorylated substrates but do not bind to such non-phosphorylated substrates (phospho-substrate antibodies) can be used to determine the presence of activated kinase in a sample.

Using certain embodiments, altered levels of activity of signal transduction pathway phosphoproteins can be associated with the prognosis of many diseases or conditions including, but not limited to neoplastic conditions associated with bone marrow.

In an embodiment, an activation state profile for a phosphoprotein of at least one signal transduction pathway is determine for a single cell. Such methods comprise providing a population of cells and sorting the population of cells by flow cytometry. In certain embodiments, cells are separated on the basis of the activation state of at least two signal transduction pathway phosphoproteins. Activation state-specific antibodies are used to sort cells on the basis of signal transduction pathway phosphoprotein activation state. In an embodiment, a multiplicity of signal transduction pathway phosphoprotein activation state antibodies are used to simultaneously determine the activation state of a multiplicity of phosphoproteins as disclosed herein. In another embodiment, cell sorting by flow cytometry on the basis of the activation state of at least two phosphoproteins as disclosed herein is combined with a determination of other flow cytometry readable outputs, such as the presence of surface markers, granularity and cell size to provide a correlation between the activation state of a multiplicity of phosphoproteins and other cell qualities measurable by flow cytometry for single cells. In certain embodiments, the presence of the cell surface markers CD34 and CD117 are used to identify cell populations having the receptor for SCF.

The present invention can also be used to determine the presence of cellular subsets, based on correlated phosphoprotein activation within complex cellular mixtures such as bone marrow hematopoietic progenitor cells or leukemic stem cells. These subsets can represent different differentiation or activation states or different cell lineages or sublineages.

It will also be recognized that a homogeneous cell population is desirable for studying signal transduction in order that variances in signaling between cells not qualitatively and quantitatively mask signal transduction events. The ultimate homogeneous system is the single cell. The present invention provides methods for the analysis of signal transduction in single cells, where the activated state of the signal transducing phosphoprotein involved is antigenically distinguishable from its non-activated state. These methods also provide for the identification of distinct signaling cascades for both artificial and stimulatory conditions in complex cell populations, such as hematopoietic progenitor (blast) cells or leukemic stem cells.

The methods provided herein can also involve the use of specific inhibitors of a signal transduction pathway. The methods provided herein can also involve the use of other pharmacological inhibitors of signaling pathways. These inhibitors can be used as controls to ensure that antibodies specifically bind to activated isoforms of a phosphoprotein. For example, an inhibitor of a signal transduction pathway phosphoprotein known to phosphorylate and activate a kinase can be used to inhibit phosphorylation of the kinase and examine whether an antibody specifically recognizes a phosphorylated isoform of the kinase. Alternatively, the inhibitors can be used to further probe signaling pathways and correlations in phosphoprotein activity, particularly in single cells. For example, inhibitors can be used to evaluate whether or not a signaling pathway is constitutively activated.

In certain embodiments, the activity of a phosphoprotein of at least one signal transduction pathway activity is determined using two or more activation state specific antibodies. In embodiments where two or more antibodies are used, the antibodies are uniquely labeled, meaning that a first activation state antibody recognizing a first signal transduction pathway phosphoprotein comprises a first label, and second activation state antibody recognizing a second signal transduction pathway phosphoprotein comprises a second label, wherein the first and second label are detectable and distinguishable, making the first antibody and the second antibody uniquely labeled. The use of a second signal transduction pathway phosphoprotein serves as an internal control to confirm specificity of the measured activity.

Although exemplified herein with regard to intracellular phosphoproteins/epitopes, the methods of the invention are equally applicable for preparation of samples aimed at measuring other post-translational modifications including, for example, ubiquitination, glycosylation, methylation, acetylation, palmitolyation, or protein-protein interactions. Thus, the invention enables the detection of non-phospho epitopes of a variety of proteins within cells, expanding the utility of the methods further. Labeled binding agents can be selected based on the particular cellular events to be studied. The methods provided by the invention allow for the examination of pathways in detailed time courses and pathway-specific manners that have previously not been available. Although diverse intracellular epitopes can be selected for flow cytometric analysis, it is understood that the user can optimize and tailor the method provided herein for the specific epitope in question by taking into account factors including, for example, localization, conformation/structure, accessibility by antibodies, and stability of the epitope. The methods provided herein are generally applicable to multicolor, multiparameter cytometry analysis.

It is understood that the steps of the assays provided herein can vary in their order. It is also understood, however, that while various options (of compounds, properties selected or order of steps) are provided herein, the options are also each provided individually, and can each be individually segregated from the other options provided herein. Moreover, steps which are obvious and known in the art that will increase the sensitivity of the assay are intended to be within the scope of this invention. For example, there can be additionally washing steps, blocking steps, etc.

In an embodiment, the reaction mixture or cells are contained in a well of a 96-well plate or other commercially available multi-well plate. In an alternate embodiment, the reaction mixture or cells are in a flow cytometry machine. Other multi-well plates useful in the present invention include, but are not limited to 384-well plates and 1536-well plates. Still other vessels for containing the reaction mixture or cells and useful in the present invention will be apparent.

The addition of the components of the assay for detecting the activation state or activity of a signal transduction pathway phosphoprotein, or inhibition of such activation state or activity, can be sequential or in a predetermined order or grouping under conditions appropriate for the activity that is assayed for. Such conditions are described here and known in the art.

In an embodiment, the methods of the invention include the use of liquid handling components. The liquid handling systems can include robotic systems comprising any number of components. In addition, any or all of the steps outlined herein can be automated; thus, for example, the systems can be completely or partially automated.

As will be appreciated there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In an embodiment, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In an embodiment, platforms for multi-well plates, multi-tubes, holders, cartridges, mini-tubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station.

In an embodiment, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

The compounds identified using the disclosed assay are potentially useful as therapeutics for many disease states including neoplastic conditions. The amount of such compound(s) will be an amount that yields the desired degree of inhibition of a signal transduction pathway phosphoprotein can generally be between 0.001 and 10000 μM.

As a matter of convenience, the method of this invention can be provided in the form of a kit. Such a kit is a packaged combination comprising the basic elements of: a) a cytokine activator of the PI3K-AKT, mTOR, RAS-MAPK, and/or JAK-STAT pathways; b) a capture molecule that binds to CD34; c) a capture molecule that binds to CD117; and d) a plurality of capture molecules that bind specifically to at least one activated phosphoprotein selected from the group consisting of pS6, pERK, pAKT, pSTAT5, and pSTAT3. In certain embodiments, the kit contains at least two capture molecules that bind at least two signal transduction pathway phosphoproteins. In certain embodiments, the kit contains at least three capture molecules that bind at least three signal transduction pathway phosphoproteins. In certain embodiments, the kit contains at least four capture molecules that bind at least four signal transduction pathway phosphoproteins. In certain embodiments, the kit contains at least five capture molecules that bind at least five signal transduction pathway phosphoproteins.

In one embodiment, the kit can further provide inhibitors of a signal transduction pathway. These inhibitors are useful to confirm that the signal transduction pathway capture molecule is specific to its target phosphoprotein and does not generally inhibit multiple functions within the cell. Additionally, these inhibitors can be used to assess whether a signal transduction pathway is constitutively activated, e.g., in an individual having or suspected of having a leukemia. In certain embodiments, the signal transduction pathway inhibitor is a MAPK pathway protein inhibitor. Several MAPK pathway protein inhibitors are commercially available and include, but are not limited to UO126, AZD6244, PD0325901, XL518, hypothemycin, anthrax lethal factor, RAF265, PLX4032, XL281, Bay 43-9006, and Zarnestra. In other embodiments, the inhibitor is a PI3K-AKT pathway inhibitor. Inhibitors of this pathway are commercially available and include, but are not limited to, rapamycin, Ly294002, and GDC-0941.

In another embodiment, the kit further comprises a solid support for the capture molecules, which can be provided as a separate element or as an element on which the capture molecules are already immobilized. Hence, the capture molecules in the kit either can be immobilized already on a solid support, or can become immobilized on a support that is included with the kit or provided separately from the kit. Where the capture molecule is labeled with an enzyme, the kit will ordinarily include substrates and cofactors required by the enzyme, where the label is a fluorophore, a dye precursor that provides the detectable chromophore, and where the label is biotin, an avidin such as avidin, streptavidin, either alone or conjugated to a chromophore. In other embodiments, the kit can further include an instruction sheet, describing how to carry out the assay of the kit.

Aspects of the present specification can be described as follows:
1. A method for determining a phosphoprotein activation profile in hematopoietic cells, the method comprising the steps of:
   a) incubating a test sample comprising hematopoietic cells with a phosphoprotein activator for at least a first incubation time period and a second incubation time period, wherein the hematopoietic cells comprise a phosphoprotein of at least one signal transduction pathway; and wherein the phosphoprotein activator is capable of activating the phosphoprotein of at least one signal transduction pathway present in the hematopoietic cells of the test sample;
   b) contacting the test sample comprising hematopoietic cells incubated for at least a first incubation time period and a second incubation time period with one or more fluorescently labeled capture molecules, the one or more fluorescently labeled capture molecules comprising at least one fluorescently labeled activated phosphoprotein capture molecule capable of binding to the phosphoprotein of at least one signal transduction pathway activated by the phosphoprotein activator; and
   c) detecting fluorescence of the one or more fluorescently labeled capture molecules from test sample comprising hematopoietic cells incubated for at least a first incubation time period and a second incubation time period; wherein the fluorescence of the at least one fluorescently labeled activated phosphoprotein capture molecule detected for the first incubation time period and the fluorescence of the at least one fluorescently labeled activated phosphoprotein capture molecule detected for the second incubation time period determines the phosphoprotein activation profile in a test sample comprising hematopoietic cells.
2. A method for determining a phosphoprotein activation profile in hematopoietic cells, the method comprising the steps of:
   a) incubating a test sample comprising hematopoietic cells with a phosphoprotein inhibitor for at least a first incubation time period and a second incubation time period, wherein the hematopoietic cells comprise a phosphoprotein of at least one signal transduction pathway; and wherein the phosphoprotein inhibitor is capable of inhibiting the phosphoprotein of at least one signal transduction pathway present in the hematopoietic cells of the test sample;
   b) contacting the test sample comprising hematopoietic cells incubated for at least a first incubation time period and a second incubation time period with one or more fluorescently labeled capture molecules, the one or more fluorescently labeled capture molecules comprising at least one fluorescently labeled activated phosphoprotein capture molecule capable of binding to the phosphoprotein of at least one signal transduction pathway inhibited by the phosphoprotein inhibitor; and
   c) detecting fluorescence of the one or more fluorescently labeled capture molecules from test sample comprising hematopoietic cells incubated for at least a first incubation time period and a second incubation time period; wherein the fluorescence of the at least one fluorescently labeled activated phosphoprotein capture molecule detected for the first incubation time period and the fluorescence of the at least one fluorescently labeled activated phosphoprotein capture molecule detected for the second incubation time period determines the phosphoprotein activation profile in a test sample comprising hematopoietic cells.
3. The embodiment of 1 or 2, wherein in step (b) the one or more fluorescently labeled capture molecules further comprises at least one fluorescently labeled control capture molecule capable of binding to a protein present in the hematopoietic cells that is not activated by the phosphoprotein activator.
4. The embodiment of 3, wherein in step (c) the fluorescence of the at least one fluorescently labeled control capture molecule detected for the first incubation time period is subtracted from the fluorescence of the at least one fluorescently labeled activated phosphoprotein capture molecule detected for the first incubation time period and the fluorescence of the at least one fluorescently labeled control capture molecule detected for the second incubation time period is subtracted from the fluorescence of the at least one fluorescently labeled activated phosphoprotein capture molecule detected for the second incubation time period in order to determine the phosphoprotein activation profile in a test sample comprising hematopoietic cells.
5. The embodiments of 1-4, wherein the test sample is from a healthy individual.
6. The embodiments of 1-4, wherein the test sample is from an individual having a disease or disorder associated with the at least one signal transduction pathway.
7. The embodiment of 6, wherein the disease or disorder associated with the at least one signal transduction pathway is a leukemia.
8. The embodiment of 7, wherein the leukemia is an acute myelogenous leukemia, an acute lymphocytic leukemia, a chronic lymphocytic leukemia, a lymphoma, a follicular lymphoma, or a multiple myeloma.
9. The embodiments of 1-4, wherein the test sample is from an individual receiving a targeted inhibitor of the at least one signal transduction pathway.
10. The embodiments of 1-9, wherein the test sample comprising hematopoietic cells is a sample from a bone marrow, a bone, a lymph node, or a cell suspension.
11. The embodiments of 1-9, wherein the hematopoietic cells comprise lymphocytes, hematopoietic progenitor cells, CD34⁺ CD117⁺ cells, CD34⁻ CD117⁺ cells, hematopoietic stem cells, leukemia stem cells, myeloid progenitor cells, granulocytes, or monocytes.
12. The embodiments of 1 and 3-11, wherein the phosphoprotein activator is a cytokine.
13. The embodiment of 12, wherein the cytokine comprises SCF, FL, IL-3, G-CSF, GM-CSF, or any combination thereof.
14. The embodiments of 1-13, wherein the at least one signal transduction pathway signal transduction pathway includes a PI3K-AKT pathway, a mTOR pathway, a RAS-MAPK pathway, a JAK-STAT pathway, or any combination thereof.
15. The embodiments of 1-14, wherein the phosphoprotein of at least one signal transduction pathway includes a S6, an ERK, an AKT, a STAT3, a STAT5, or any combination thereof.
16. The embodiments of 1-15, wherein in step (a) the first incubation time period and the second incubation time period are each for about 0.5 minute to about 60 minutes.
17. The embodiments of 1-15, wherein in step (a) the first incubation time period and the second incubation time period are each for about 2 minutes to about 30 minutes.
18. The embodiments of 1-17, wherein in step (a) separate aliquots of the test sample comprising hematopoietic cells are incubated for the first incubation time period and the second incubation time period or the same aliquot of the test sample comprising hematopoietic cells is incubated for the first incubation time period and the second incubation time period.
19. The embodiments of 1-18, wherein the at least one fluorescently-labeled phosphoprotein capture molecule includes a fluorescently-labeled pS6 capture molecule, a fluorescently-labeled pERK capture molecule, a fluorescently-labeled pAKT capture molecule, a fluorescently-labeled pSTAT3 capture molecule, a fluorescently-labeled pSTAT5 capture molecule, or any combination thereof.
20. The embodiments of 3-19, wherein the at least one fluorescently-labeled control capture molecule includes a fluorescently-labeled CD34 capture molecule, a fluorescently-labeled CD45 capture molecule, a fluorescently-labeled CD117 capture molecule, any combination thereof.
21. The embodiments of 1-20, wherein in step (c) detecting is accomplished by cytometry.
22. The embodiments of 1-21, wherein in step (c) the fluorescence of the one or more fluorescently labeled capture molecules for at least a first incubation time period and a second incubation time period detected is analyzed as an area under the curve, a frequency of positive stained cells, a ratio of positive stained cells to negative stained cells, a mean fluorescence intensity, a median fluorescence intensity, a mode fluorescence intensity, or the time/duration of a positive response.
23. The embodiments of 1-22, wherein the phosphoprotein activation profile determined in step (c) is indicative of a disease or condition.
24. The embodiments of 1-23, further comprising evaluating the phosphoprotein activation profile determined in step (c) to a phosphoprotein activation profile determined in a reference sample comprising hematopoietic cells, wherein the reference sample comprising hematopoietic cells is a sample not incubated with a phosphoprotein activator for at least a first incubation time period and a second incubation time period.
25. The embodiment of 24, wherein the reference sample is a second aliquot of the test sample comprising hematopoietic cells or a standardized reference sample.
26. The embodiments of 1 and 3-25, further comprising incubating the test sample comprising hematopoietic cells with an inhibitor prior to incubating the test sample comprising hematopoietic cells with the phosphoprotein activator, wherein the inhibitor is capable of inhibiting the activation of a phosphoprotein of at least one signal transduction pathway present in the hematopoietic cells of the test sample.
27. The embodiments of 2-26, wherein the inhibitor is UO126, AZD6244, PD0325901, XL518, hypothemycin, anthrax lethal factor, RAF265, PLX4032, XL281, Bay 43-9006, Zarnestra, rapamycin, Ly294002, GDC-0941, or any combination thereof.
28. The embodiments of 1-27, further comprising before step (b) the step of preserving the test sample comprising hematopoietic cells with a preservative.
29. The embodiment of 28, wherein the preservative is an aldehyde, a paraformaldehyde, a formaldehyde, or any combination thereof.
30. The embodiments of 1-29, further comprising treating the preserved hematopoietic cells in the test sample with an permealizing agent.
31. The embodiment of 30, wherein permealizing agent comprises a detergent.
32. The embodiment of 31, wherein the detergent is added at a concentration of between about 0.1% (v/v) and about 10% (v/v).
33. The embodiment of 31 or 32, wherein the detergent is Triton X-100, Nonidet P-40 (NP-40), or Brij-58.
34. The embodiments of 1-33, further comprising treating the preserved hematopoietic cells in the test sample with an unmasking agent.
35. The embodiment of 34 wherein unmasking agent comprises an alcohol.
36. The embodiment of 35, wherein the alcohol is added at a concentration of between about 25% (v/v) and about 90% (v/v).
37. The embodiment of 36 or 37, wherein the alcohol is ethanol or methanol.
38. A method for detecting leukemia, the method comprising the steps of:
   a) incubating a test sample comprising hematopoietic cells and a reference sample comprising hematopoietic cells with a phosphoprotein activator, wherein the test sample is obtained from an individual having or suspected of having a leukemia; wherein the reference sample is obtained from an individual not having or not suspected of having a leukemia; wherein the hematopoietic cells of the test sample and the reference sample comprise a phosphoprotein of at least one signal transduction pathway; and wherein the phosphoprotein activator is capable of activating the phosphoprotein of at least one signal transduction pathway present in the hematopoietic cells of the test sample;
   b) contacting the test sample comprising hematopoietic cells and a reference sample comprising hematopoietic cells with one or more fluorescently labeled capture molecules, wherein the one or more fluorescently labeled capture molecules comprise at least one fluorescently labeled activated phosphoprotein capture molecule capable of binding to the phosphoprotein of at least one signal transduction pathway activated by the phosphoprotein activator;
   c) detecting fluorescence of the one or more fluorescently labeled capture molecules present in the test sample and the reference sample; and
   d) comparing the fluorescence detected for the test sample comprising hematopoietic cells to the fluorescence detected for the reference sample comprising hematopoietic cells, wherein a difference in the fluorescence detected for the test sample comprising hematopoietic cells relative to the fluorescence detected for the reference sample comprising hematopoietic cells is indicative of the leukemia.
39. A method for detecting leukemia, the method comprising the steps of:
   a) incubating a test sample comprising hematopoietic cells and a reference sample comprising hematopoietic cells with a phosphoprotein inhibitor, wherein the test sample is obtained from an individual having or suspected of having a leukemia; wherein the reference sample is obtained from an individual not having or not suspected of having a leukemia; wherein the hematopoietic cells of the test sample and the reference sample comprise a phosphoprotein of at least one signal transduction pathway; and wherein the phosphoprotein inhibitor is capable of inhibiting the phosphoprotein of at least one signal transduction pathway present in the hematopoietic cells of the test sample;
   b) contacting the test sample comprising hematopoietic cells and a reference sample comprising hematopoietic cells with one or more fluorescently labeled capture molecules, wherein the one or more fluorescently labeled capture molecules comprise at least one fluorescently labeled activated phosphoprotein capture molecule capable of binding to the phosphoprotein of at least one signal transduction pathway inhibited by the phosphoprotein inhibitor;
   c) detecting fluorescence of the one or more fluorescently labeled capture molecules present in the test sample and the reference sample; and
   d) comparing the fluorescence detected for the test sample comprising hematopoietic cells to the fluorescence detected for the reference sample comprising hematopoietic cells, wherein a difference in the fluorescence detected for the test sample comprising hematopoietic cells relative to the fluorescence detected for the reference sample comprising hematopoietic cells is indicative of the leukemia.
40. The embodiment of 38 or 39, wherein in step (b) the one or more fluorescently labeled capture molecules further comprises at least one fluorescently labeled control capture molecule capable of binding to a protein present in the hematopoietic cells that is not activated by the phosphoprotein activator.
41. The embodiment of 40, wherein in step (c) the fluorescence of the at least one fluorescently labeled control capture molecule detected in the test sample is subtracted from the fluorescence of the at least one fluorescently labeled activated phosphoprotein capture molecule detected in the test sample and the fluorescence of the at least one fluorescently labeled control capture molecule detected in the reference sample is subtracted from the fluorescence of the at least one fluorescently labeled activated phosphoprotein capture molecule detected in the reference sample in order to determine the fluorescence detected for the test sample and the reference sample.
42. The embodiments of 38-41, wherein the leukemia is an acute myelogenous leukemia, an acute lymphocytic leukemia, a chronic lymphocytic leukemia, a lymphoma, a follicular lymphoma, or a multiple myeloma.
43. The embodiments of 38-42, wherein the test sample is from an individual receiving a targeted inhibitor of the at least one signal transduction pathway.
44. The embodiments of 38-43, wherein the test sample is from an individual receiving a leukemia treatment.
45. The embodiments of 38-44, wherein the test sample comprising hematopoietic cells is a sample from a bone marrow, a bone, a lymph node, or a cell suspension.
46. The embodiments of 38-45, wherein the hematopoietic cells comprise lymphocytes, hematopoietic progenitor cells, CD34⁺ CD117⁺ cells, CD34⁻ CD117⁺ cells, hematopoietic stem cells, leukemia stem cells, myeloid progenitor cells, granulocytes, or monocytes.
47. The embodiments of 38-46, wherein the reference sample comprising hematopoietic cells is a sample from a bone marrow, a bone, a lymph node, or a cell suspension.
48. The embodiments of 38-47, wherein the hematopoietic cells comprise lymphocytes, hematopoietic progenitor cells, CD34⁺ CD117⁺ cells, CD34⁻ CD117⁺ cells, hematopoietic stem cells, leukemia stem cells, myeloid progenitor cells, granulocytes, or monocytes.
49. The embodiments of 38 and 40-48, wherein the phosphoprotein activator is a cytokine.
50. The embodiment of 49, wherein the cytokine comprises SCF, FL, IL-3, G-CSF, GM-CSF, or any combination thereof.
51. The embodiments of 38-50, wherein the at least one signal transduction pathway signal transduction pathway includes a PI3K-AKT pathway, a mTOR pathway, a RAS-MAPK pathway, a JAK-STAT pathway, or any combination thereof.
52. The embodiments of 38-51, wherein the phosphoprotein of at least one signal transduction pathway includes a S6, an ERK, an AKT, a STAT3, a STAT5, or any combination thereof.
53. The embodiments of 38-52, wherein in step (a) incubation of the test sample and the reference sample are each for about 0.5 minute to about 60 minutes.
54. The embodiments of 38-52, wherein in step (a) incubation of the test sample and the reference sample are each for about 2 minutes to about 30 minutes.
55. The embodiments of 38-54, wherein the at least one fluorescently-labeled phosphoprotein capture molecule includes a fluorescently-labeled pS6 capture molecule, a fluorescently-labeled pERK capture molecule, a fluorescently-labeled pAKT capture molecule, a fluorescently-labeled pSTAT3 capture molecule, a fluorescently-labeled pSTAT5 capture molecule, or any combination thereof.
56. The embodiments of 38-55, wherein the at least one fluorescently-labeled control capture molecule includes a fluorescently-labeled CD34 capture molecule, a fluorescently-labeled CD45 capture molecule, a fluorescently-labeled CD117 capture molecule, any combination thereof.
57. The embodiments of 38-56, wherein in step (c) detecting is accomplished by cytometry.
58. The embodiments of 38-57, wherein in step (c) the fluorescence detected is analyzed as an area under the curve, a frequency of positive stained cells, a ratio of positive stained cells to negative stained cells, a mean fluorescence intensity, a median fluorescence intensity, a mode fluorescence intensity, or the time/duration of a positive response.
59. The embodiments of 38 and 40-58, further comprising incubating the test sample comprising hematopoietic cells and the reference sample comprising hematopoietic cells with an inhibitor prior to incubating the test sample comprising hematopoietic cells and the reference sample comprising hematopoietic cells with the phosphoprotein activator, wherein the inhibitor is capable of inhibiting the activation of a phosphoprotein of at least one signal transduction pathway present in the hematopoietic cells of the test sample.
60. The embodiment of 39-59, wherein the inhibitor is UO126, AZD6244, PD0325901, XL518, hypothemycin, anthrax lethal factor, RAF265, PLX4032, XL281, Bay 43-9006, Zarnestra, rapamycin, Ly294002, GDC-0941, or any combination thereof.
61. A method for detecting a signal transduction activation state in an individual having or suspected of having a disease or condition associated with activation of a signal transduction pathway, the method comprising the steps of:
   a) determining a phosphoprotein activation profile of at least one signal transduction pathway from a hematopoietic cell population in a test sample, the test sample obtained from an individual having or suspected of having a disease or condition associated with activation of a signal transduction pathway;
   b) determining a phosphoprotein activation profile of at least one signal transduction pathway from a hematopoietic cell population in a reference sample, the reference sample obtained from an individual not having or not suspected of having a disease or condition associated with activation of a signal transduction pathway, wherein the phosphoprotein activation profile of at least one signal transduction pathway measured from the test sample and the reference sample is the same; and
   c) comparing the phosphoprotein activation profile measured in step (a) with the phosphoprotein activation profile measured in step (b), wherein identifying a difference in the phosphoprotein activation profile measured in step (a) from the phosphoprotein activation profile measured in step (b) is indicative of the disease or condition associated with activation of a signal transduction pathway.
62. The embodiment of 61, wherein determining a phosphoprotein activation profile of step (a) and step (b) is performed according to the embodiments of 1-37.
63. The embodiment of 61 or 62, wherein the disease or condition is leukemia.
64. The embodiments of 61-63, further comprising repeating step (a) with a test sample from the individual after the individual has received a therapeutic agent to treat the disease or condition and monitoring the effectiveness of that therapeutic agent by monitoring for a change between the activation profile from the individual before and after treatment.
65. The embodiments of 61-64, wherein the test sample is from a patient receiving a targeted inhibitor of a signaling pathway.
66. A method for detecting a leukemia, the method comprising the steps of:
   a) determining a phosphoprotein activation profile of at least one signal transduction pathway from a hematopoietic cell population in a test sample, the test sample obtained from an individual having or suspected of having a leukemia;
   b) determining a phosphoprotein activation profile of at least one signal transduction pathway from a hematopoietic cell population in a reference sample, the reference sample obtained from an individual not having or not suspected of having a leukemia, wherein the phosphoprotein activation profile of at least one signal transduction pathway measured from the test sample and the reference sample is the same; and
   c) comparing the phosphoprotein activation profile measured in step (a) with the phosphoprotein activation profile measured in step (b), wherein identifying a difference in the phosphoprotein activation profile measured in step (a) from the phosphoprotein activation profile measured in step (b) is indicative of the leukemia.
67. The embodiment of 66, wherein determining a phosphoprotein activation profile of step (a) and step (b) is performed according to the embodiments of 1-37.
68. The embodiment of 66 or 67, further comprising repeating step (a) with a sample from the individual after the individual has received a therapeutic agent to treat the leukemia and monitoring the effectiveness of that therapeutic agent by monitoring for a change between the activation profile from the individual before and after treatment.
69. A kit for determining a phosphoprotein activation profile in a sample containing hematopoietic cells, the kit comprising:
   a) a cytokine activator of a PI3K-AKT pathway, a mTOR pathway, a RAS-MAPK pathway, a JAK-STAT pathway, or any combination thereof;
   b) a CD34 capture molecule;
   c) a CD117 capture molecule; and
   d) one or more of phosphoprotein capture molecules, the one or more phosphoprotein capture molecules comprising a pS6 capture molecule, a pERK capture molecule, a pAKT capture molecule, a pSTAT3 capture molecule, a pSTAT5 capture molecule, or any combination thereof.
70. The embodiment of 69, wherein the cytokine activator is a SCF, a FL, a IL-3, a IL-27, a GM-CSF, or any combination thereof.
71. The embodiment of 69 or 70, wherein the CD34 capture molecule is a fluorescently-labeled CD34 capture molecules or a chemiluminescent label CD34 capture molecules.
72. The embodiments of 69-71, wherein the CD117 capture molecule is a fluorescently-labeled CD117 capture molecules or a chemiluminescent label CD117 capture molecules.
73. The embodiments of 69-72, wherein the one or more phosphoprotein capture molecules comprise one or more fluorescently-labeled phosphoprotein capture molecules or one or more chemiluminescent label phosphoprotein capture molecules.
74. The embodiment of 73, wherein the one or more fluorescently-labeled phosphoprotein capture molecules comprise a fluorescently-labeled pS6 capture molecule, a fluorescently-labeled pERK capture molecule, a fluorescently-labeled pAKT capture molecule, a fluorescently-labeled pSTAT3 capture molecule, a fluorescently-labeled pSTAT5 capture molecule, or any combination thereof.
75. The embodiments of 68-74, wherein the CD34 capture molecule, the CD34 capture molecule, and the one or more phosphoprotein capture molecules comprise one or more antibodies or antigen binding fragments thereof.
76. The embodiments of 69-75, further comprising one or more inhibitors of one or more signal transduction pathways, one or more signal transduction pathways including a PI3K-AKT pathway, mTOR pathway, RAS-MAPK pathway, JAK-STAT pathway, or any combination thereof.
77. The embodiment of 76, wherein the one or more inhibitors is UO126, AZD6244, PD0325901, XL518, hypothemycin, anthrax lethal factor, RAF265, PLX4032, XL281, Bay 43-9006, Zarnestra, rapamycin, Ly294002, GDC-0941, or any combination thereof.
78. A method for detecting a leukemia, the method comprising the steps of:
   a) incubating a test sample comprising hematopoietic cells and a reference sample comprising hematopoietic cells with a SCF, wherein the test sample is obtained from an individual having or suspected of having a leukemia; wherein the reference sample is obtained from an individual not having or not suspected of having a leukemia; wherein the hematopoietic cells of the test sample and the reference sample comprise an ERK; and wherein the SCF is capable of activating the ERK present in the hematopoietic cells of the test sample and the reference sample;
   b) contacting the test sample comprising hematopoietic cells and the reference sample comprising hematopoietic cells with one or more fluorescently labeled capture molecules, wherein the one or more fluorescently labeled capture molecules comprise at least one fluorescently labeled activated pERK capture molecule capable of binding to the ERK activated by the SCF;
   c) detecting fluorescence of the one or more fluorescently labeled capture molecules present in the test sample and the reference sample; and
   d) comparing the fluorescence detected for the test sample comprising hematopoietic cells to the fluorescence detected for the reference sample comprising hematopoietic cells, wherein a 1-fold or more increase in the fluorescence for activated ERK detected in the test sample comprising hematopoietic cells relative to the fluorescence for activated ERK detected in the reference sample comprising hematopoietic cells is indicative of the leukemia.
79. A method for detecting a leukemia, the method comprising the steps of:
   a) incubating a test sample comprising hematopoietic cells and a reference sample comprising hematopoietic cells with a FL, wherein the test sample is obtained from an individual having or suspected of having a leukemia; wherein the reference sample is obtained from an individual not having or not suspected of having a leukemia; wherein the hematopoietic cells of the test sample and the reference sample comprise an ERK; and wherein the FL is capable of activating the ERK present in the hematopoietic cells of the test sample and the reference sample;
   b) contacting the test sample comprising hematopoietic cells and the reference sample comprising hematopoietic cells with one or more fluorescently labeled capture molecules, wherein the one or more fluorescently labeled capture molecules comprise at least one fluorescently labeled activated pERK capture molecule capable of binding to the ERK activated by the FL;
   c) detecting fluorescence of the one or more fluorescently labeled capture molecules present in the test sample and the reference sample; and
   d) comparing the fluorescence detected for the test sample comprising hematopoietic cells to the fluorescence detected for the reference sample comprising hematopoietic cells, wherein a 1-fold or more increase in the fluorescence for activated ERK detected in the test sample comprising hematopoietic cells relative to the fluorescence for activated ERK detected in the reference sample comprising hematopoietic cells is indicative of the leukemia.
80. A method for detecting a leukemia, the method comprising the steps of:
   a) incubating a test sample comprising hematopoietic cells and a reference sample comprising hematopoietic cells with a SCF, wherein the test sample is obtained from an individual having or suspected of having a leukemia; wherein the reference sample is obtained from an individual not having or not suspected of having a leukemia; wherein the hematopoietic cells of the test sample and the reference sample comprise a S6; and wherein the SCF is capable of activating the S6 present in the hematopoietic cells of the test sample and the reference sample;
   b) contacting the test sample comprising hematopoietic cells and the reference sample comprising hematopoietic cells with one or more fluorescently labeled capture molecules, wherein the one or more fluorescently labeled capture molecules comprise at least one fluorescently labeled activated pS6 capture molecule capable of binding to the S6 activated by the SCF;
   c) detecting fluorescence of the one or more fluorescently labeled capture molecules present in the test sample and the reference sample; and
   d) comparing the fluorescence detected for the test sample comprising hematopoietic cells to the fluorescence detected for the reference sample comprising hematopoietic cells, wherein a 10-fold or more increase in the fluorescence for activated S6 detected in the test sample comprising hematopoietic cells relative to the fluorescence for activated S6 detected in the reference sample comprising hematopoietic cells is indicative of the leukemia.
81. A method for detecting a leukemia, the method comprising the steps of:
   a) incubating a test sample comprising hematopoietic cells and a reference sample comprising hematopoietic cells with a IL-3, wherein the test sample is obtained from an individual having or suspected of having a leukemia; wherein the reference sample is obtained from an individual not having or not suspected of having a leukemia; wherein the hematopoietic cells of the test sample and the reference sample comprise a STAT5; and wherein the IL-3 is capable of activating the STAT5 present in the hematopoietic cells of the test sample and the reference sample;
   b) contacting the test sample comprising hematopoietic cells and the reference sample comprising hematopoietic cells with one or more fluorescently labeled capture molecules, wherein the one or more fluorescently labeled capture molecules comprise at least one fluorescently labeled activated pSTAT5 capture molecule capable of binding to the STAT5 activated by the IL-3;
   c) detecting fluorescence of the one or more fluorescently labeled capture molecules present in the test sample and the reference sample; and
   d) comparing the fluorescence detected for the test sample comprising hematopoietic cells to the fluorescence detected for the reference sample comprising hematopoietic cells, wherein a 1-fold or more increase in the fluorescence for activated STAT5 detected in the test sample comprising hematopoietic cells relative to the fluorescence for activated STAT5 detected in the reference sample comprising hematopoietic cells is indicative of the leukemia.
82. A method for detecting a leukemia, the method comprising the steps of:
   a) incubating a test sample comprising hematopoietic cells and a reference sample comprising hematopoietic cells with a GM-CSF, wherein the test sample is obtained from an individual having or suspected of having a leukemia; wherein the reference sample is obtained from an individual not having or not suspected of having a leukemia; wherein the hematopoietic cells of the test sample and the reference sample comprise a STAT5; and wherein the GM-CSF is capable of activating the STAT5 present in the hematopoietic cells of the test sample and the reference sample;
   b) contacting the test sample comprising hematopoietic cells and the reference sample comprising hematopoietic cells with one or more fluorescently labeled capture molecules, wherein the one or more fluorescently labeled capture molecules comprise at least one fluorescently labeled activated pSTAT5 capture molecule capable of binding to the STAT5 activated by the GM-CSF;
   c) detecting fluorescence of the one or more fluorescently labeled capture molecules present in the test sample and the reference sample; and
   d) comparing the fluorescence detected for the test sample comprising hematopoietic cells to the fluorescence detected for the reference sample comprising hematopoietic cells, wherein a 1-fold or more increase in the fluorescence for activated STAT5 detected in the test sample comprising hematopoietic cells relative to the fluorescence for activated STAT5 detected in the reference sample comprising hematopoietic cells is indicative of the leukemia.
83. A method for detecting a leukemia, the method comprising the steps of:
   a) incubating a test sample comprising hematopoietic cells and a reference sample comprising hematopoietic cells with a SCF, wherein the test sample is obtained from an individual having or suspected of having a leukemia; wherein the reference sample is obtained from an individual not having or not suspected of having a leukemia; wherein the hematopoietic cells of the test sample and the reference sample comprise an ERK and an AKT; and wherein the SCF is capable of activating both the ERK and the AKT present in the hematopoietic cells of the test sample and the reference sample;
   b) contacting the test sample comprising hematopoietic cells and the reference sample comprising hematopoietic cells with one or more fluorescently labeled capture molecules, wherein the one or more fluorescently labeled capture molecules comprise at least one fluorescently labeled activated pERK capture molecule capable of binding to the ERK activated by the SCF and at least one fluorescently labeled activated pAKT capture molecule capable of binding to the AKT activated by the SCF;
   c) detecting fluorescence of the one or more fluorescently labeled capture molecules present in the test sample and the reference sample; and
   d) comparing the fluorescence detected for the test sample comprising hematopoietic cells to the fluorescence detected for the reference sample comprising hematopoietic cells, wherein an increase in the fluorescence for activated ERK and activated AKT detected in the reference sample comprising hematopoietic cells, but only an increase in the fluorescence for activated AKT detected in the test sample comprising hematopoietic cells is indicative of the leukemia.
84. A method for detecting a leukemia, the method comprising the steps of:
   a) contacting a test sample comprising hematopoietic cells and a reference sample comprising hematopoietic cells with one or more fluorescently labeled capture molecules, wherein the test sample is obtained from an individual having or suspected of having a leukemia; wherein the reference sample is obtained from an individual not having or not suspected of having a leukemia; wherein the one or more fluorescently labeled capture molecules comprise at least one fluorescently labeled activated pS6 capture molecule capable of binding phosphorylated S6, at least one fluorescently labeled activated pAKT capture molecule capable of binding phosphorylated AKT, at least one fluorescently labeled activated pSTAT5 capture molecule capable of binding phosphorylated STAT5, or any combination thereof;
   b) detecting fluorescence of the one or more fluorescently labeled capture molecules present in the test sample and the reference sample; and
   c) comparing the fluorescence detected for the test sample comprising hematopoietic cells to the fluorescence detected for the reference sample comprising hematopoietic cells, wherein an increase in the fluorescence for phosphorylated S6, phosphorylated AKT or phosphorylated STAT5 detected in the test sample comprising hematopoietic cells relative to the fluorescence for phosphorylated S6, phosphorylated AKT or phosphorylated STAT5 detected in the reference sample comprising hematopoietic cells is indicative of the leukemia.
85. A method for detecting a leukemia, the method comprising the steps of:
   a) incubating a test sample comprising hematopoietic cells and a reference sample comprising hematopoietic cells with a GM-CSF, wherein the test sample is obtained from an individual having or suspected of having a leukemia; wherein the reference sample is obtained from an individual not having or not suspected of having a leukemia; wherein the hematopoietic cells of the test sample and the reference sample comprise an AKT; and wherein the GM-CSF is capable of activating the AKT present in the hematopoietic cells of the test sample and the reference sample;
   b) contacting the test sample comprising hematopoietic cells and the reference sample comprising hematopoietic cells with one or more fluorescently labeled capture molecules, wherein the one or more fluorescently labeled capture molecules comprise at least one fluorescently labeled activated pAKT capture molecule capable of binding to the AKT activated by the GM-CSF;
   c) detecting fluorescence of the one or more fluorescently labeled capture molecules present in the test sample and the reference sample; and
   d) comparing the fluorescence detected for the test sample comprising hematopoietic cells to the fluorescence detected for the reference sample comprising hematopoietic cells, wherein an increase in the fluorescence for activated AKT detected in the test sample comprising hematopoietic cells relative to the fluorescence for activated AKT detected in the reference sample is indicative of the leukemia.
86. A method for detecting a leukemia, the method comprising the steps of:
   a) incubating a test sample comprising hematopoietic cells and a reference sample comprising hematopoietic cells with a SCF for at least a first incubation time period and a second incubation time period, wherein the test sample is obtained from an individual having or suspected of having a leukemia; wherein the reference sample is obtained from an individual not having or not suspected of having a leukemia; wherein the hematopoietic cells of the test sample and the reference sample comprise an AKT; and wherein the SCF is capable of activating the AKT present in the hematopoietic cells of the test sample and the reference sample;
   b) contacting the test sample comprising hematopoietic cells and the reference sample comprising hematopoietic cells incubated for at least the first incubation time period and the second incubation time period with one or more fluorescently labeled capture molecules, wherein the one or more fluorescently labeled capture molecules comprise at least one fluorescently labeled activated pAKT capture molecule capable of binding to the AKT activated by the SCF;
   c) detecting fluorescence of the one or more fluorescently labeled capture molecules present in the test sample and the reference sample incubated for at least the first incubation time period and the second incubation time period; and
   d) comparing the fluorescence detected for the test sample comprising hematopoietic cells to the fluorescence detected for the reference sample comprising hematopoietic cells incubated for at least the first incubation time period and the second incubation time period to determine an AKT phosphorylation rate in the test sample and an AKT phosphorylation rate in the reference sample, wherein a lower AKT phosphorylation rate detected in the test sample relative to the AKT phosphorylation rate detected in the reference sample is indicative of the leukemia.

### EXAMPLES

### Example 1

### Phosphoprotien Response of Immunophenotypically Defined Cell Populations from Normal Bone Marrow to Growth Factor/Cytokine Stimulation

Bone marrow (BM) samples were collected in 7.5 mL Iscove's modified Dulbecco's medium (IMDM) supplemented with 100U penicillin, 0.01 mg/mL streptomycin, and 70 μg/mL heparin sodium salt from 10 patients undergoing hip replacement surgery, who were otherwise healthy. These patients were from a pool of surgery patients, age 49 to 77, and represented 7 female and 8 male patients. Wright Giemsa stained slides prepared from these samples were examined by Hematopathologist (AC) to confirm that no underlying hematological disorders were present. These BM samples were rinsed twice with IMDM to release cells, filtered using a 100 μm Nylon cell strainer to remove marrow particles, and centrifuged for at RT fro 7 minutes at 200 g. The cell pellet was washed twice with sterile IMDM to remove traces of fat and re-suspended in IMDM to a concentration of 3×10⁷ cells/mL.

To assess the signal transduction response after growth factor simulation, a series comprising two sets of tubes including about 3×10⁶ cells/100 μL were incubated at 37° C. for 30 minutes. After this pre-incubation period, tubes with then processes with either a stimulant or inhibitor. For signal stimulation, tubes were incubated at 2 minutes, 3.5 minutes, 7 minutes, 15 minutes, and 30 minutes with one of the following cytokines to stimulate protein phosphorylation: 10 ng/100 μL SCF, 50 ng/100 μL FL, 10 ng/100 μL GM-CSF, 10 ng/100 μL IL-3, or 10 ng/100 μL G-CSF. To examine the effects of protein phosphorylation inhibition, tubes were incubated with an inhibitor cocktail comprising 100 μM/100 μL UO126, 1 μM/100 μL LY294002, and 1 μM/100 μL rapamycin. One unstimulated tube receiving no stimulant or inhibitor was processed to assess basal levels of phospho-protein expression. After stimulation the cells were immediately fixed in 10% formaldehyde solution at 37° C. Pre-labeling with CD13, CD16 and CD64 antibodies was carried out 20 minutes prior to formaldehyde fixation. After fixation, one set of tubes was permeabilized without methanol for pAKT, pERK and pS6 staining and the other for 80% methanol permeabilized for pSTAT3 and pSTAT5 staining. After permeablization, the cells were washed in PBS with 2% BSA and blocked by re-suspended in 75 μL of PBS comprising 25% heat inactivated normal mouse serum, 25% normal rabbit serum. To stain cells with fluorescent conjugated monoclonal antibodies, samples were incubated on ice for 45 minutes in 100 μL of an antibody staining solution including either: 1) CD15-Pacific Orange, CD34-Phycoerythrin-Texas Red, CD117-Phycoerythrin-Cyanin 5.5, CD45-Allophycocyanin-Alexa Fluor 750 (or CD45-Pacific Orange, in which case CD15-Pacific Orange was omitted), pS6-Pacific Blue, pERK-Alexa Fluor 488 and pAKT-Alexa Fluor 647 or 2) CD15-Pacific Orange, CD34-Phycoerythrin-Texas Red, CD117-Phycoerythrin-Cyanin 5.5, CD45-Allophycocyanin-Alexa Fluor 750 or CD45-Pacific Blue, pSTAT3-Alexa Fluor 488 and pSTAT5-Phycoerythrin. The stained cells were washed twice with ice cold PBS containing 2% BSA and centrifuged at 800 g for 6 minutes at 4° C. The supernatant was discarded and cells re-suspended in 250 μL of chilled wash buffer for data acquisition. Stained cells were examined using either GALIOS flow cytometer (Beckman Coulter) or CYAN ADP (Beckman Coulter) equipped with 488 nm, 405 nm and 635 nm lasers to detect the degree of fluorescence. Data were analyzed using FCS Express (De Novo Software, CA) analysis software.

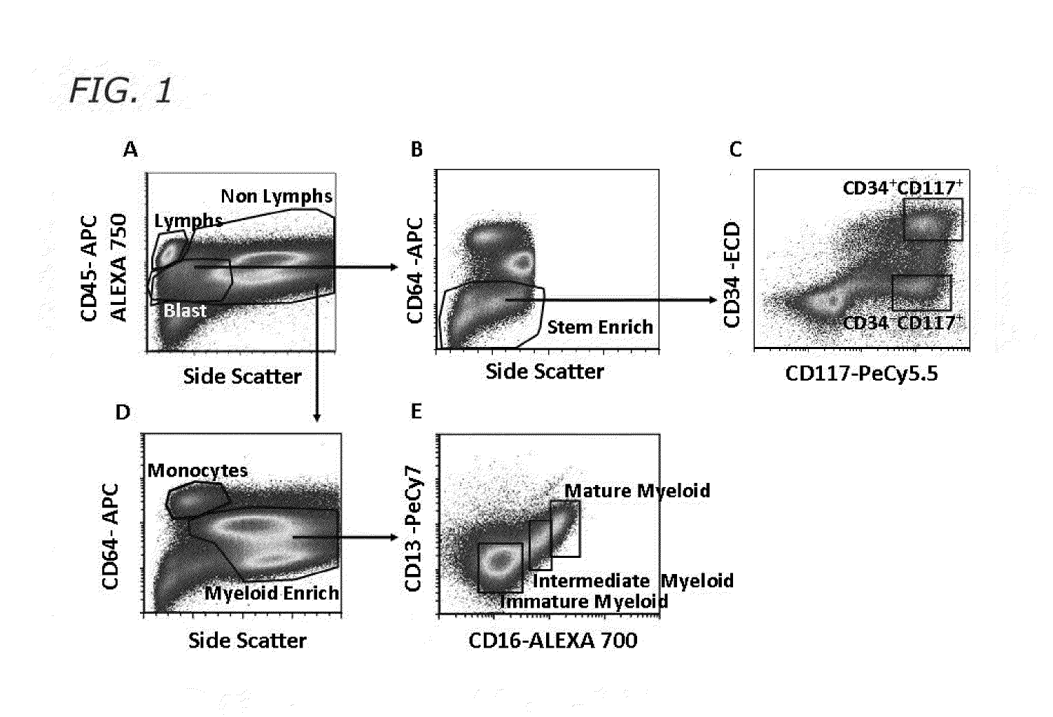

Specific phosphoprotein expression of pERK, pS6, pAKT, pSTAT5, and pSTAT3 was examined in seven discrete phenotypically defined populations based on 7-color immunophenotyping (FIG. 1). The percent of responding (phosphorprotein positive) cells for each of these populations was determined from individual single parameter histograms based on a fixed integration region set on the inhibitor treated control at about 3% positives. For responding populations in which the phosphoprotein fluorescent staining distribution showed limited or no overlap with inhibitor treated distribution, the median fluorescence intensity of the total population was used for calculation of responding signal to noise (S/N) (median fluorescence intensity of the responding population divided by the median fluorescence intensity of the lymphocytes for that particular sample). For populations exhibiting a bimodal response, the phosphoprotein fluorescence intensity median of the responding cells was taken for only the responding cell peak. In the rare occasion where the responding population showed significant overlap with the inhibitor treated control, S/N of the response was not calculated.

Tables 1-5 summarizes the signaling responses of pERK, pS6, pAKT, pSTAT3 and pSTAT5 in the six different cell populations of normal BM with SCF, FL, GM-CSF, IL-3 and G-CSF stimulation. The tables include the mean and the standard deviation of the percent responding cell population as well as the signal/noise (S/N) ratio for the 5 different phosphoproteins in response to the above-mentioned stimuli in each of these populations. Treatment of the lymphocytes with either cytokines/growth factors or inhibitors showed no modulation of the phosphoprotein epitopes studied (pERK, pS6, pAKT, pSTAT3 or pSTAT5) or the background fluorescence levels. The range of arbitrary MFI of the lymphocyte population for pERK, pS6, pAKT, pSTAT3 and pSTAT5 were 2.3-4.1 (CV=22.5%), 3.4-8.5 (CV=33.6%), 7.1-10.2 (CV=12.7%), 2.7-4.6 (CV=18.9%), and 2.6-4.9 (CV=22.5%) respectively. Lymphocytes thus served as a de facto internal negative control and their median fluorescence intensity (MFI) was used for normalization of the phosphoprotein measurements.

Stimulation of normal BM with SCF resulted in up-regulation of pERK, pAKT and pS6 showing percent responding of 89.8%, 58.0% and 81.7% in the CD34⁺ blast population, respectively (S/N of 13.3, 7.5 and 48.4, respectively) (Table 1). Similarly, activation of pERK (67.7%), pAKT (60.6%) and pS6 (51.9%) was seen in the CD34⁻CD117⁺ population, although the magnitude of response was slightly lower than seen in the CD34⁺ cells. The phospho-protein response distributions were homogeneous with the exception of pS6 response in the CD34⁻CD117⁺ cells which had a fraction positive of 53.8% responding to SCF. No pSTAT3 or pSTAT5 activation was seen in these two populations. As expected with SCF stimulation, no response was seen in the other immature or mature granulocyte and monocyte populations.

TABLE 1
SCF-Stimulated Phosphorylation in Immunophenotypically Defined Cell Populations
from Healthy Donor BM Samples

| Phospho- | | Percent (%) Responding Cells² | | | | | |
|---|---|---|---|---|---|---|---|
| protein | Parameter¹ | CD34⁺ | CD117⁺ | G1 | G2 | G3 | Mono |
| pERK | Mean | 89.8 ± 6.9 | 67.7 ± 20.1 | 1.6 ± 0.8 | 2.8 ± 2.1 | 6.5 ± 7.2 | 13.6 ± 3.6 |
| | S/N ratio | 13.3 ± 4.9 | 8.6 ± 3.2 | 2.9 ± 0.6 | 2.7 ± 0.5 | 3.1 ± 0.6 | 3.2 ± 2.1 |
| pS6 | Mean | 81.7 ± 16.3 | 51.9 ± 16.9 | 2.1 ± 1.4 | 2.1 ± 1.2 | 3.8 ± 2.6 | 43.5 ± 19.9 |
| | S/N ratio | 48.4 ± 39.3 | 108.2 ± 94.9 | 3.3 ± 1.9 | 2.9 ± 1.8 | 3.5 ± 2.3 | 11.2 ± 12.3 |
| pAKT | Mean | 58.0 ± 27.3 | 60.6 ± 33.5 | 2.9 ± 2.4 | 3.9 ± 3.9 | 4.2 ± 3.0 | 6.5 ± 8.2 |
| | S/N ratio | 7.5 ± 5.7 | 7.3 ± 5.4 | 2.2 ± 1.1 | 2.3 ± 1.0 | 2.6 ± 1.2 | 2.4 ± 1.0 |
| pSTAT3 | Mean | 7.3 ± 4.3 | 2.6 ± 1.4 | 3.0 ± 3.5 | 2.2 ± 1.8 | 4.7 ± 4.0 | 4.4 ± 2.0 |
| | S/N ratio | 1.9 ± 0.2 | 2.5 ± 0.6 | 3.3 ± 0.5 | 3.3 ± 0.5 | 3.6 ± 0.6 | 2.4 ± 0.3 |
| pSTAT5 | Mean | 11.6 ± 3.0 | 7.4 ± 4.7 | 6.8 ± 8.0 | 4.9 ± 5.6 | 6.9 ± 6.7 | 9.5 ± 13.8 |
| | S/N ratio | 1.5 ± 0.9 | 2.2 ± 0.7 | 3.1 ± 0.7 | 3.2 ± 0.7 | 3.8 ± 0.8 | 2.5 ± 0.6 |

¹Mean values are scaled 0 to 100%. S/N ration values are scaled to highest value seen for each phosphoprotein.
²CD34⁺, CD34⁺/CD117⁺ cells; CD117⁺, CD34⁻/CD117⁺ cells; G1, immature myeloid cells; G2, intermediate myeloid cells; G3, mature myeloid cells; and Mono, monocytes as defined in gating scheme shown in FIG. 1.

FL stimulation showed homogeneous activation of pERK and pS6 with percent responding of 76.9% and 68.8%, respectively, but no activation of pSTAT3 or pSTAT5 in normal CD34⁺ blast cells; a response similar to SCF stimulation (Table 2). But in contrast to SCF, pAKT response with FL stimulation was heterogeneous with a discrete fraction responding of 34.3%. In comparison to SCF stimulation, with FL, the pERK, pAKT, and pS6 percent responding in the CD34⁻CD117⁺ blast population was lower and the magnitude (S/N) of response reduced in the latter two. No activation was seen in the other granulocyte populations. However, uupregulation of pERK and pS6 with percent responding of approximately 68% was seen in the monocytes (S/N of 12.8 and 33.4, respectively) as was a small percent responding of 21.5% for pAKT. As with SCF, no activation of pSTAT3 or pSTAT5 was seen with FL stimulation in any cell populations of these normal BMs.

TABLE 2
FL-Stimulated Phosphorylation in Immunophenotypically Defined Cell Populations from
Healthy Donor BM Samples

| Phospho- | | Percent (%) Responding Cells² | | | | | |
|---|---|---|---|---|---|---|---|
| protein | Parameter¹ | CD34⁺ | CD117⁺ | G1 | G2 | G3 | Mono |
| pERK | Mean | 76.9 ± 11.0 | 37.1 ± 8.8 | 3.3 ± 5.4 | 6.3 ± 11.2 | 9.1 ± 15.6 | 67.4 ± 18.2 |
| | S/N ratio | 11.9 ± 3.6 | 10.9 ± 2.9 | 2.9 ± 0.7 | 2.8 ± 0.6 | 3.6 ± 1.3 | 12.8 ± 6.7 |
| pS6 | Mean | 68.8 ± 21.1 | 36.1 ± 16.4 | 2.1 ± 1.5 | 2.5 ± 1.9 | 3.9 ± 2.7 | 67.6 ± 23.7 |
| | S/N ratio | 31.1 ± 26.0 | 44.4 ± 44.4 | 3.3 ± 1.9 | 2.9 ± 1.8 | 3.6 ± 2.3 | 33.4 ± 30.5 |
| pAKT | Mean | 34.3 ± 19.4 | 20.5 ± 28.3 | 3.0 ± 4.3 | 3.6 ± 5.0 | 3.8 ± 2.5 | 21.5 ± 23.8 |
| | S/N ratio | 7.2 ± 4.9 | 2.8 ± 2.9 | 2.3 ± 1.1 | 2.4 ± 1.0 | 2.7 ± 1.3 | 4.2 ± 3.0 |
| pSTAT3 | Mean | 13.5 ± 9.4 | 3.5 ± 2.2 | 2.7 ± 3.3 | 1.8 ± 1.4 | 5.3 ± 5.6 | 4.8 ± 3.9 |
| | S/N ratio | 3.5 ± 3.3 | 2.6 ± 0.6 | 3.4 ± 0.4 | 3.3 ± 0.6 | 3.7 ± 0.5 | 2.6 ± 0.4 |
| pSTAT5 | Mean | 16.1 ± 6.1 | 5.7 ± 5.8 | 2.7 ± 2.5 | 2.0 ± 1.6 | 3.1 ± 1.0 | 3.1 ± 1.9 |
| | S/N ratio | 1.9 ± 1.4 | 2.1 ± 0.6 | 2.6 ± 0.5 | 2.7 ± 0.5 | 3.2 ± 0.7 | 3.9 ± 4.9 |

¹Mean values are scaled 0 to 100%. S/N ration values are scaled to highest value seen for each phosphoprotein.
²CD34⁺, CD34⁺/CD117⁺ cells; CD117⁺, CD34⁻/CD117⁺ cells; G1, immature myeloid cells; G2, intermediate myeloid cells; G3, mature myeloid cells; and Mono, monocytes as defined in gating scheme shown in FIG. 1.

With GM-CSF stimulation, heterogeneous, bimodal, pERK (fraction positive μ=30.1%) and pS6 (μ=60.1%) responses were seen in the CD34⁺blast populations in most samples. No pAKT was detected in these cells (Table 3). pSTAT5 was homogeneously upregulated in the CD34⁺ blasts (74.7% responding), but no upregulation of pSTAT3 was seen in the blast and monocyte populations with GM-CSF stimulation. In contrast, the CD34⁻CD117⁺ cells showed lower pERK stimulation with a percent responding of about 30% and a pSTAT5 S/N of about 15 fold which was also lower compared to the CD34⁺ blast population. Upregulation of pERK was seen in the immature and mature granulocytes and in the monocytes with percent responding of 53.9%, 91.5% and 90.7%, respectively. pSTAT3 was upregulated in the immature and mature granulocytes with percent responding of 58.7% and 59.1%, respectively, but not in the monocytes. Although pAKT showed little upregulation in the monocytes, pS6 was activated. pSTAT5 was robustly activated in the immature, mature granulocyte and monocyte (percent responding all above 90%) populations. A steady increase in magnitude (S/N) of pSTAT5 signal in response to GM-CSF was observed with increasing myeloid maturation rising from 6.2 to 11.3.

TABLE 3
GM-CSF-Stimulated Phosphorylation in Immunophenotypically Defined Cell Populations
from Healthy Donor BM Samples

| Phospho- | | Percent (%) Responding Cells² | | | | | |
|---|---|---|---|---|---|---|---|
| protein | Parameter¹ | CD34⁺ | CD117⁺ | G1 | G2 | G3 | Mono |
| pERK | Mean | 30.1 ± 11.5 | 26.2 ± 13.9 | 53.9 ± 20.8 | 91.5 ± 16.0 | 90.7 ± 15.0 | 52.0 ± 22.6 |
| | S/N ratio | 10.6 ± 4.4 | 3.8 ± 0.4 | 6.2 ± 1.8 | 9.4 ± 2.4 | 11.3 ± 3.1 | 11.4 ± 4.5 |
| pS6 | Mean | 60.1 ± 14.1 | 38.5 ± 11.5 | 3.4 ± 1.3 | 11.3 ± 15.9 | 8.3 ± 7.0 | 76.9 ± 11.8 |
| | S/N ratio | 35.7 ± 31.7 | 52.7 ± 44.0 | 5.1 ± 3.2 | 5.7 ± 3.7 | 5.7 ± 3.6 | 25.8 ± 20.8 |
| pAKT | Mean | 22.5 ± 23.4 | 13.3 ± 23.7 | 11.2 ± 19.8 | 17.6 ± 26.1 | 17.5 ± 26.0 | 17.6 ± 27.3 |
| | S/N ratio | 3.2 ± 3.3 | 2.2 ± 1.3 | 3.0 ± 1.6 | 3.2 ± 1.6 | 3.7 ± 1.9 | 3.6 ± 2.3 |
| pSTAT3 | Mean | 10.4 ± 7.9 | 7.9 ± 8.7 | 58.7 ± 39.9 | 59.1 ± 30.4 | 76.0 ± 30.8 | 6.0 2.7 |
| | S/N ratio | 1.7 ± 0.2 | 2.6 ± 0.8 | 6.8 ± 2.5 | 7.3 ± 2.1 | 8.8 ± 2.7 | 2.3 ± 0.3 |
| pSTAT5 | Mean | 74.7 ± .6.2 | 58.2 ± 11.8 | 98.2 ± 1.4 | 98.8 ± 0.4 | 98.9 ± 0.5 | 83.2 ± 28.1 |
| | S/N ratio | 19.8 ± 9.7 | 15.3 ± 4.6 | 16.8 ± 3.8 | 18.3 ± 5.5 | 24.1 ± 6.3 | 14.7 ± 4.5 |

¹Mean values are scaled 0 to 100%. S/N ration values are scaled to highest value seen for each phosphoprotein.
²CD34⁺, CD34⁺/CD117⁺ cells; CD117⁺, CD34⁻/CD117⁺ cells; G1, immature myeloid cells; G2, intermediate myeloid cells; G3, mature myeloid cells; and Mono, monocytes as defined in gating scheme shown in FIG. 1.

Activation of pERK (S/N=12.2) in the CD34⁺ blast population (percent responding of 26%) was seen along with pS6 and pSTAT5 percent responding of 33% and 76%, respectively, following IL-3 stimulation (Table 4). Although similar signaling patterns, in the monocytes percent responding was higher than in the CD34⁺ blasts but magnitude of response was lower (S/N in monocytes of pERK, pS6 and pSTAT5: 11.4, 26.0 and 13.5, respectively). The granulocyte populations showed some upregulation of pERK (percent responding 25.6% in immature to 41.1% in mature) but no activation of pS6. As with GM-CSF stimulation, pSTAT5 showed increasing magnitude (S/N) with granulocyte maturation. The magnitude of the IL-3 response was however lower in comparison to GM-CSF stimulation respectively in the immature (13.8 versus 16.8) and mature (17.7 versus 24.1) granulocytes. No upregulation of pAKT or pSTAT3 was observed in any of the populations with IL-3 stimulation.

TABLE 4
IL3-Stimulated Phosphorylation in Immunophenotypically Defined Cell Populations from
Healthy Donor BM Samples

| Phospho- | | Percent (%) Responding Cells² | | | | | |
|---|---|---|---|---|---|---|---|
| protein | Parameter¹ | CD34⁺ | CD117⁺ | G1 | G2 | G3 | Mono |
| pERK | Mean | 26.2 ± 9.1 | 10.0 ± 7.1 | 25.6 ± 26.2 | 47.4 ± 30.0 | 41.1 ± 18.1 | 41.8 ± 19.4 |
| | S/N ratio | 12.2 ± 3.5 | 2.7 ± 0.6 | 4.3 ± 1.6 | 5.7 ± 2.4 | 6.2 ± 2.3 | 11.4 ± 4.7 |
| pS6 | Mean | 50.7 ± 11.3 | 33.2 ± 12.3 | 2.7 ± 1.5 | 4.2 ± 3.3 | 5.5 ± 3.1 | 71.4 ± 10.5 |
| | S/N ratio | 39.5 ± 31.2 | 60.3 ± 49.5 | 4.5 ± 2.4 | 4.1 ± 2.3 | 4.7 ± 2.6 | 26.0 ± 21.0 |
| pAKT | Mean | 11.8 ± 11.0 | 3.7 ± 4.0 | 3.4 ± 3.4 | 5.3 ± 5.2 | 5.6 ± 4.1 | 9.6 ± 14.6 |
| | S/N ratio | 1.8 ± 0.8 | 1.9 ± 1.0 | 2.5 ± 1.2 | 2.6 ± 1.1 | 2.9 ± 1.4 | 3.1 ± 1.7 |
| pSTAT3 | Mean | 9.3 ± 6.1 | 5.6 ± 4.3 | 6.2 ± 5.2 | 4.6 ± 2.7 | 7.5 ± 5.6 | 4.9 ± 2.9 |
| | S/N ratio | 1.9 ± 0.4 | 2.6 ± 0.6 | 3.3 ± 0.6 | 3.3 ± 0.7 | 3.7 ± 0.7 | 2.3 ± 0.4 |
| pSTAT5 | Mean | 76.0 ± 7.5 | 59.3 ± 16.0 | 96.6 ± 3.1 | 98.7 ± 0.7 | 98.2 ± 1.4 | 90.7 ± 4.3 |
| | S/N ratio | 19.3 ± 8.9 | 12.4 ± 2.0 | 13.8 ± 3.0 | 14.2 ± 3.2 | 17.7 ± 3.8 | 13.5 ± 4.0 |

¹Mean values are scaled 0 to 100%. S/N ration values are scaled to highest value seen for each phosphoprotein.
²CD34⁺, CD34⁺/CD117⁺ cells; CD117⁺, CD34⁻/CD117⁺ cells; G1, immature myeloid cells; G2, intermediate myeloid cells; G3, mature myeloid cells; and Mono, monocytes as defined in gating scheme shown in FIG. 1.

G-CSF showed activation of pERK, pS6, pAKT, pSTAT3 and pSTAT5 at varying levels across the cell populations studied (Table 5). Response in CD34⁺ blast population for pERK and pAKT were heterogeneous with 47.1% and 22.5% discrete fractions of responding cells, respectively. The pERK response in the CD34⁺ blasts was more robust (10.2 S/N) than in the other G-CSF responding populations (S/N range 3.2 to 6.8). Monocytes showed limited to no response to G-CSF stimulation. In contrast, granulocyte populations showed homogeneous pERK activation to varying levels (percent responding 22.9% in immature to 46.0% in mature). pSTAT3 response in these populations was homogenous and robust with a percent responding of about 90% and S/N>13, higher than that seen with any other growth factor stimulation studied. In contrast, percent responding (about 70%) and magnitude of the pSTAT5 response (S/N 8.7 to 11.7) was lower in the granulocyte subsets studied compared to either GM-CSF (16.8 to 24.1) or IL-3 (13.8 to 17.7) stimulation. The magnitude of G-CSF stimulated pSTAT5 in the CD34⁺ blasts (25.2) was higher than the other populations and, as with other stimuli, showed increasing response with granulocyte maturation. No expression of pS6 or pAKT was seen within the granulocytes. However, the CD34⁺ blasts and monocytes showed some low levels of pS6 (percent responding of 52% and 42.2% respectively).

TABLE 5
G-CSF-Stimulated Phosphorylation in Immunophenotypically Defined Cell Populations
from Healthy Donor BM Samples

| Phospho- | | Percent (%) Responding Cells² | | | | | |
|---|---|---|---|---|---|---|---|
| protein | Parameter¹ | CD34⁺ | CD117⁺ | G1 | G2 | G3 | Mono |
| pERK | Mean | 47.1 ± 12.6 | 39.2 ± 13.9 | 22.9 ± 24.1 | 55.2 ± 33.1 | 46.0 ± 30.3 | 12.7 ± 8.8 |
| | S/N ratio | 10.2 ± 2.7 | 6.8 ± 2.5 | 4.2 ± 1.6 | 5.8 ± 2.4 | 6.2 ± 2.2 | 3.2 ± 1.6 |
| pS6 | Mean | 52.0 ± 34.2 | 41.5 ± 26.5 | 2.7 ± 1.9 | 2.9 ± 2.3 | 4.2 ± 3.0 | 42.2 ± 30.2 |
| | S/N ratio | 30.7 ± 25.2 | 12.9 ± 10.1 | 4.0 ± 2.3 | 3.7 ± 2.1 | 4.0 ± 2.4 | 17.2 ± 16.8 |
| pAKT | Mean | 22.5 ± 18.4 | 16.6 ± 19.8 | 5.3 ± 10.9 | 4.1 ± 3.3 | 4.4 ± 3.1 | 4.1 ± 4.9 |
| | S/N ratio | 6.4 ± 5.9 | 2.7 ± 1.8 | 2.5 ± 1.5 | 2.5 ± 1.2 | 2.8 ± 1.4 | 2.6 ± 1.2 |
| pSTAT3 | Mean | 77.8 ± 19.1 | 46.2 ± 26.8 | 91.2 ± 20.4 | 91.3 ± 19.3 | 89.2 ± 24.9 | 27.8 ± 31.8 |
| | S/N ratio | 9.8 ± 3.0 | 9.9 ± 4.6 | 13.3 ± 4.8 | 13.3 ± 5.1 | 14.0 ± 5.5 | 6.1 ± 2.7 |
| pSTAT5 | Mean | 82.6 ± 14.0 | 49.8 ± 25.2 | 70.9 ± 26.5 | 71.6 ± 22.0 | 71.4 ± 17.7 | 29.5 ± 19.9 |
| | S/N ratio | 25.2 ± 13.3 | 17.0 ± 11.9 | 8.7 ± 1.9 | 9.7 ± 2.3 | 11.7 ± 3.0 | 5.9 ± 2.0 |

¹Mean values are scaled 0 to 100%. S/N ration values are scaled to highest value seen for each phosphoprotein.
²CD34⁺, CD34⁺/CD117⁺ cells; CD117⁺, CD34⁻/CD117⁺ cells; G1, immature myeloid cells; G2, intermediate myeloid cells; G3, mature myeloid cells; and Mono, monocytes as defined in gating scheme shown in FIG. 1.

### Example 2

### Analysis of Growth Factor/Cytokine Responsiveness of a Cell Population with the Presence of its Cognate Receptor

To characterize immunophenotype and growth factor receptor expression of cell samples, three tubes each containing about 3×10⁶ cells were incubated with 2 mL of NH₄Cl-based red blood cell (RBC) lysing solution at RT for 5 minutes. The incubated cells were centrifuged at RT fro 5 minutes at 200 g, and the resulting cell pellets washed twice with PBS and blocked by re-suspended in 75 μL of PBS comprising 25% heat inactivated normal mouse serum, 25% normal rabbit serum. To stain cells with fluorescent conjugated monoclonal antibodies, samples were incubated at 4° C. for 30 minutes in 100 μL of an antibody staining solution including the following fluorescent-labeled antibodies directed to membrane antigens: CD11b-Pacific Blue, CD15-Pacific Orange, CD34-Phycoerythrin-Texas Red, CD117-Phycoerythrin-Cyanin 5.5, CD13-Phycoerythrin-Cyanin 7, CD64-Allophycocyanin, CD16-Alexa Fluor 700 and CD45-Allophycocyanin-Alexa Fluor 750. Of these, one tube served as the Phycoerythrin fluorescence-minus-one (FMO) no antibody control to ensure proper gating, and the remaining 2 tubes were incubated with CD114-Phycoerythrin, CD115 or CD135-Phycoerythrin. Single color compensation controls were also processed to create a compensation matrix that was applied to all samples. The stained cells were washed twice with ice cold PBS containing 2% BSA and centrifuged at 800 g for 6 minutes at 4° C. The supernatant was discarded and cells re-suspended in 250 μL of chilled wash buffer for data acquisition. Cells were examined using either Galios flow cytometer (Beckman Coulter) or CyAn ADP (Beckman Coulter) equipped with 488 nm, 405 nm and 635 nm lasers. Data were analyzed using FCS Express (De Novo Software, CA) analysis software.

This analysis indicates that for some populations, there was a clear homogeneous signaling response indicative of the entre population of cells responding to a given stimuli. This homogeneous response was reflective of a homogeneous receptor expression pattern in these cell populations. For example, homogeneous receptor staining was seen for both the G-CSF receptor (CD114) and the GM-CSF receptor (CD116) in the monocyte and all granulocyte populations, and homogenous responses were seen to stimulation with either of these growth factors. Conversely, lymphocytes showed no response to G-CSF stimulation and these cells do not express the G-CSF receptor (CD114⁻). Additionally, for some populations, there were clear bimodal signaling responses indicative of only a subset of cells responding to a given stimulus. For example, there were bimodal responses to G-CSF and FL with clear responding and non-responding subpopulations in the CD34⁺CD117⁺ population. A bimodal response to G-CSF was also seen in the CD34⁻CD117⁺ subset where bimodal CD114 expression was also seen. In general, this was reflective of growth factor receptor expression in responding subpopulations. For example, there was a biomodal expression pattern of the G-CSF receptor (CD114) and FLT-3 receptor (CD135) in CD34⁺CD117⁺ population. Subsequent analysis demonstrated a relatively good correlation (y=0.9227x−6.2009; r²=0.89) between percent CD34⁺CD117⁺ cells staining positive for the G-CSF receptor (CD114⁺) and the percent CD34⁺CD117⁺ cells responding to G-CSF stimulation as assessed by pERK phosphorylation. These results indicate that there was a clear correlation between the responsiveness of a cell population for a growth factor or cytokine and the presence of its cognate receptor.

### Example 3

### Phosphoprotien Response of Immunophenotypically Defined Cell Populations from AML Bone Marrow to Growth Factor/Cytokine Stimulation

Using normal BM signaling profiles of Example 1 as comparison, analysis of 14 AML samples was carried out to identify differences in signaling responses. White blood cells from BM or peripheral blood (PB) samples were obtained from 14 diagnosed AML patients undergoing routine clinical flow cytometry analysis using IRB approved protocols. The AML patients included newly diagnosed AML, AMLs arising in a background of MDS, therapy related AMLs and previously diagnosed AML post therapy. The median age of the patients at diagnosis was 51 years (range: 29-75 years) and represented 5 female and 9 male patients. The median white blood cell was 6.4 million/mL (range: 0.9-106 M/mL) and the BM morphologic blast count was an average of 46% (range: 0%-90%). The abnormal cell immunophenotype and gene mutation (FLT-3 and NPM1) status are summarized in Table 6.

TABLE 6
Characteristics of AML Patients

| | WBC | | | | | | | | | |
|---|---|---|---|---|---|---|---|---|---|---|
| | Count | BM Differential (%)² | | | | | | | Final | Genetic |
| Patient¹ | (M/mL) | B | GP | EP | M | L | E | Immunophenotype | Diagnosis³ | mutations |
| AML1 | 3.4 | 28 | 40 | 1 | 2 | 21 | 8 | CD117⁺, CD34⁻, CD33⁺, MPO⁺, | AML-M2 | FLT3ITD⁺ |
| (BM) | | | | | | | | CD13^{dim}, CD64^{dim}, CD11b⁻, | relapse | |
| | | | | | | | | HLADR⁺ | | |
| AML2 | 13.7 | 73 | 11 | 0 | 1 | 16 | 0 | CD34⁺, CD13⁺, CD33^{dim}. MPO⁻, | AML arising | Negative |
| (PB) | | | | | | | | CD117⁺, CD64⁻, CD11b⁻, CD7^{+,} | from MPN | |
| | | | | | | | | HLADR⁺, CD19^{partial+}, TdT⁺ | | |
| AML3 | 6 | 31 | 28 | 14 | 0 | 6 | 20 | CD34⁺, CD64⁻, MPO⁺, CD7⁺, | AML-M1 | FLT3ITD⁺ |
| (BM) | | | | | | | | CD117^{dim}, CD33⁺, CD11b⁻, | relapse | |
| | | | | | | | | CD13⁻, HLADR⁺ | | |
| AML4 | 2.9 | 10 | 14 | 6 | 6 | 9 | 53 | CD34⁺, CD13⁺, CD33⁺, MPO⁻, | AML | Negative |
| (BM) | | | | | | | | CD117^{dim}, CD11b⁻, CD64−, | relapse, | |
| | | | | | | | | HLADR⁺ | 10% blasts | |
| AML5 | 87.3 | 66 | 4 | 14 | 3 | 7 | 2 | CD34⁺, CD13⁺, CD33^{dim}, MPO⁺, | AML- | Negative |
| (BM) | | | | | | | | CD117⁺, CD11b⁻, CD14⁻, | M1/M2 | |
| | | | | | | | | CD64^{dim partial} | (NSE+ rare | |
| | | | | | | | | | blast) | |
| AML6 | 5 | 32 | 25 | 0 | 7 | 35 | 0 | CD34⁺, CD117⁺, CD13⁺, CD7⁺, | AML arising | Negative |
| (PB) | | | | | | | | CD14⁻, CD33⁺, MPO^{dim}, CD15⁻, | from MDS | |
| | | | | | | | | HLADR⁺, CD64^{dim}, CD11b⁻ | | |
| AML7 | 5.3 | 0 | 57 | 2 | 4 | 4 | 33 | CD34⁺, CD117⁺, CD13⁺, MPO⁺, | Normocellular, | Negative |
| (BM) | | | | | | | | CD33⁺ | no | |
| | | | | | | | | | leukemia | |
| | | | | | | | | | evidence, | |
| | | | | | | | | | tAML | |
| | | | | | | | | | history | |
| AML8 | 6.7 | 20 | 34 | 1 | 3 | 10 | 31 | CD34⁺, CD13⁺, CD33⁺, CD64⁻, | AML | Negative |
| (BM) | | | | | | | | CD117^{dim}, MPO⁻, CD11b⁻, | relapse, 15-20% | |
| | | | | | | | | HLADR⁺ | blasts | |
| AML9 | 12.9 | 60 | 19 | 5 | 1 | 5 | 9 | CD34⁺, CD117⁺, CD13⁺, MPO⁺, | AML-M2 | NPM1⁺ |
| (BM) | | | | | | | | CD33⁺, CD64^{dim}, CD14⁻, | | |
| | | | | | | | | CD11b^{dim}, CD7^{dim} | | |
| AML10 | 0.9 | 84 | 1 | 1 | 1 | 3 | 1 | CD34⁺, CD117⁺, CD13⁺, CD64⁻, | tAML | Negative |
| (BM) | | | | | | | | CD33^{dim}, MPO^{dim}, HLADR⁺, | relapse | |
| | | | | | | | | CD11b⁻ | | |
| AML11 | 7.7 | 90 | 1 | 0 | 0 | 6 | 3 | CD34⁺, CD117⁺, CD13⁺, CD33⁺, | AML | Negative |
| (BM) | | | | | | | | MPO^{dim/-}, HLADR⁺, CD64⁻, | relapse | |
| | | | | | | | | CD11b⁻, CD7^{partial} | | |
| AML12 | 1.5 | 11 | 58 | 3 | 1 | 10 | 17 | CD34⁺, CD13⁺, CD33⁺, MPO⁺, | AML | — |
| (BM) | | | | | | | | CD117⁺, HLADR⁺, CD64⁻, | relapse with | |
| | | | | | | | | CD11b⁺, CD14⁻, CD7⁺ | MDS | |
| | | | | | | | | | related | |
| | | | | | | | | | changes | |
| AML13 | 54.5 | 90 | 0 | 1 | 1 | 8 | 1 | CD34⁺, CD117⁺, CD13⁻, MPO⁺, | AML M2 | Negative |
| (BM) | | | | | | | | CD33⁺, HLADR⁺, CD64⁻, CD3⁻, | relapse | |
| | | | | | | | | CD11b⁻, CD2⁺, CD7⁺, CD19^{dim}, | | |
| | | | | | | | | CD10⁻ | | |
| AML14 | 106 | 68 | 8 | 1 | 0 | 16 | 5 | CD34⁺, CD64⁻, CD13⁺, CD33⁻, | AML M2 | — |
| (BM) | | | | | | | | CD117⁺, MPO^{dim}, HLADR⁺ | relapse | |

¹BM, Bone marrow; PB, Peripheral blood
²B, Blasts; GP, Granulocyte precursor; EP, Eosinophil precursor; E, Erythrocyte precursor; M, Monocytes; L, Lymphocytes.
³MPO, Myeloperoxidase; MPN, Myeloproliferative neoplasm; MDS, Myelodysplastic syndrome; FLT3ITD, FLT-3 internal tandem duplication; NPM1, nucleophosmin 1.

Experiments performed to assess the signal transduction response after growth factor simulation were performed as described for Example 1.

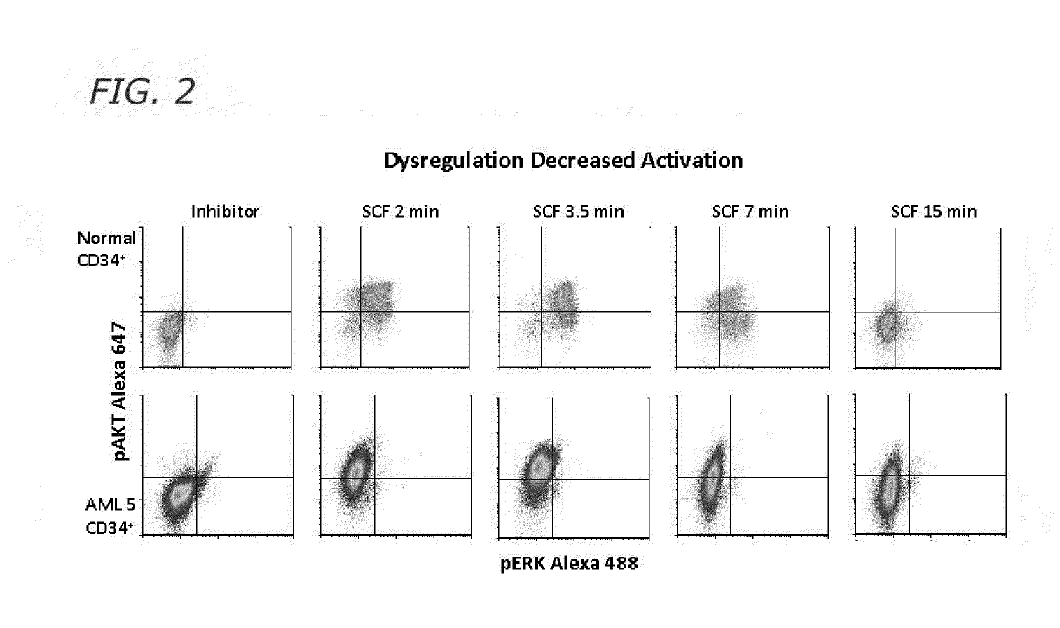

Easily identifiable alterations in downstream signaling response were seen across all growth factor/cytokine pathways and phospho-proteins studied in 12 of the 14 AML samples analyzed when compared to responses in immunophenotypically similar normal BM cell subsets, with specific alterations varying from patient to patient and population to population. Further, when adequate numbers of cells were available for testing, multiple abnormalities were identified per patient, although it is important to note that a subset of responses were also normal. A representative subset of the clear, overt alterations seen are summarized in Table 7. FIG. 2 shows pERK signaling following SCF stimulation as one example of dysregulated, loss of response in AML cells as compared to cells obtained from a health individual.

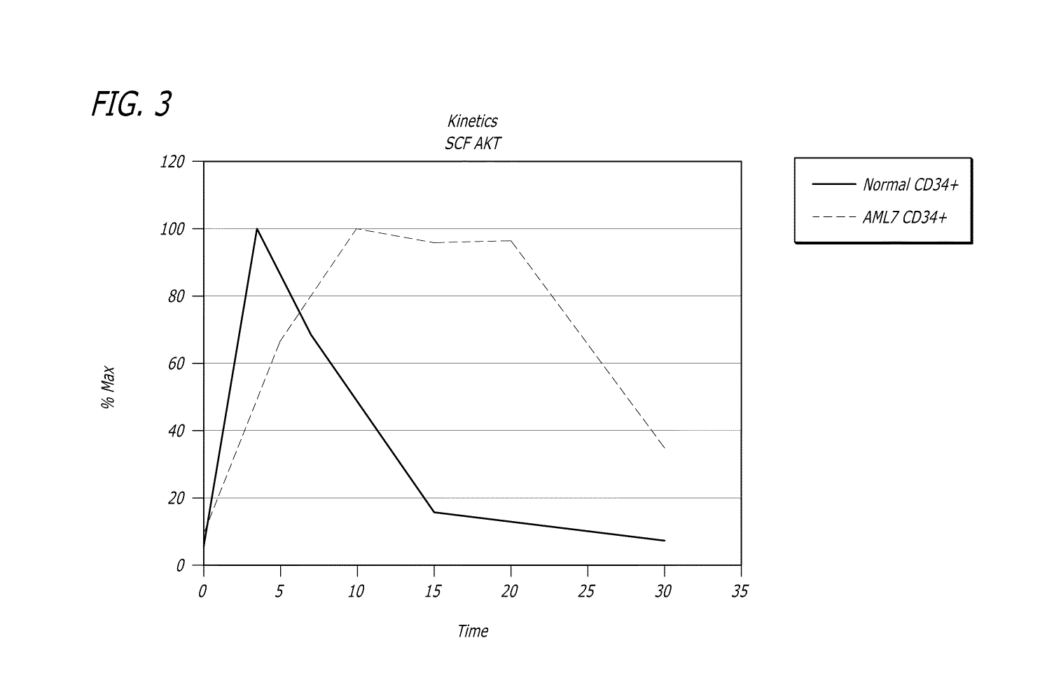

Summarizing across all patients, the differences seen spanned a wide spectrum and included altered kinetics of activation, increased basal, or constitutive, levels of phospho-protein expression and abnormal responses to cytokine/growth factor stimulation, either reduced or enhanced activation. For example, in cells from normal individuals, SCF leads to upregulation of pERK and pAKT in the CD34⁺ cells. However in AML5, SCF stimulation led to the typical upregulation of pAKT in the CD34⁺ cells, but no pERK upregulation was seen. In this example, the positive pAKT response serves as an internal control documenting presence of functional receptor. Another example of abnormal signaling patterns is shown by the analysis of AML1. AML1 showed constitutive level of pSTAT5 phosphorylation (S/N=7.7) in the CD34⁻CD117⁺ blast population, however, none of the normal BMs analyzed showed significant basal levels of pSTAT5 in the CD34⁻CD117⁺ myeloid cells. Increased basal levels of pS6 and pAKT (data not shown) were also observed in some patient samples. Analysis of phosphorproteins in AML9 versus normal cells shows differences in signaling downstream of the GM-CSF receptor that illustrate activation of a target not normally seen. Typically, there is no pAKT upregulation in normal CD34⁺ cells after GM-CSF stimulation, but in contrast, AML9 showed a measurable increase in pAKT in the CD34⁺ blasts following GM-CSF stimulation. AML7 illustrates another important type of difference observed between normal and the AML samples, namely altered kinetics of phosphorprotein activation following stimulation. AML7 showed significant delayed activation, and prolonged expression, of pAKT in the abnormal blasts in response to SCF as compared to normal (FIG. 3).

TABLE 7
Cytokine-Stimulated Phosphorylation in AML Cell Populations

| | Growth | | | |
|---|---|---|---|---|
| Patient¹ | Factor² | Phosphoprotein | Normal Sample | AML Sample |
| AML1 | Constitutive | pSTAT5 | 3.0 | 95.7 |
| | FL | pERK | 76.9 | 10.3 |
| AML2 | SCF | pERK | 89.8 | 4.8 |
| | GM-CSF | pSTAT5 | 75.1 | 14.6 |
| | Constitutive | pS6 | 5.7 | 49.6 |
| AML3 | G-CSF | pSTAT3 | 77.8 | 7.0 |
| | G-CSF | pSTAT5 | 82.6 | 8.3 |
| AML5 | SCF | pERK | 13.3 | 3.4 |
| | SCF | pERK | 89.8 | 11.9 |
| | FL | pERK | 76.9 | 8.1 |
| | FL | pAKT | 76.9 | 8.1 |
| | GM-CSF | pSTAT5 | 74.7 | 2.0 |
| AML6 | GM-CSF | pSTAT5 | 74.7 | 2.5 |
| | SCF | pERK | 89.8 | 3.0 |
| AML7 | SCF | pAKT | — | — |
| AML8 | IL-3 | pERK | 26.2 | 57.3 |
| | GM-CSF | pSTAT5 | 74.7 | 10.6 |
| | Constitutive | pS6 | 5.7 | 61.4 |
| AML9 | GM-CSF | pAKT | 16.2 | 49.2 |
| | FL | pERK | 76.9 | 12.0 |
| AML11 | SCF | pERK | 89.8 | 3.3 |
| | SCF | pAKT | 58.0 | 3.2 |
| AML12 | SCF | pERK | 89.8 | 3.3 |
| AML13 | GM-CSF | pSTAT5 | 74.7 | 14.6 |
| | SCF | pERK | 89.8 | 4.8 |
| AML14 | GM-CSF | pSTAT5 | 74.7 | 4.7 |

¹Samples from AML4 and AML10 yielded insufficient cell numbers to perform experiments.
²Constitutive represents an altered basal stimulation (untreated sample) where the signal transduction cascade phosphorylates phosphoproteins in the absence of growth factor/cytokine stimulation.

### Example 4

### Phosphoprotein Activation Profiles in Bone Marrow Samples from Healthy Donor Individuals

Phosphoprotein kinetic profiles were analyzed in bone marrow aspiration samples from nine healthy, adult donors (i.e., normal bone marrow or NBM) and from five AML patients. Samples from healthy donors included 5 males and 4 females, ranging in age from 26 to 49 years. Approximately 10 to 20 mL of bone marrow (BM) samples were collected from the posterior iliac crest of the volunteers using IRB approved protocols. The BM samples were filtered using a 40 μm Nylon cell strainer to remove marrow particles and the white blood cell concentration determined using a blood analyzer. The samples contained approximately 7 to 31 million white blood cells (WBCs) per mL. The samples were immediately used for subsequent experimentation.

To assess the signal transduction response after growth factor simulation, 100 to 200 μL of processed samples including about 2×10⁶ WBC/100 μL were added to tubes and incubated at 37° C. for 30 minutes. For inhibition experiments, cells were incubated with 100 μM U0126, 1 μg/mL rapamycin, or both for at least 20 minutes prior to cytokine addition and in all cases for 30 min prior to formaldehyde addition. After this pre-incubation period, tubes were incubated at 0, 1, 2, 4, 6, 8, 10, 15, 20, 25, 30, and 60 minutes with one of the following cytokines to stimulate protein phosphorylation: 100 ng/mL SCF, 25 ng/mL FL, 25 ng/mL GM-CSF, or 25 ng/mL IL-3. After stimulation the cells were immediately fixed by the addition of formaldehyde to a final concentration of 4% v/v and incubated for 10 minutes at room temperature. Following fixation, red blood cells were lysed by the addition of TRITON-X/PBS solution (0.1% v/v final concentration Triton X-100) supplemented with a 1 x phosphatase inhibitor cocktail (PIC; final concentrations: 0.2 mM sodium orthovanadate, 2 mM sodium pyrophosphate decahydrate, 2 mM [3-glycerophosphate, and 10 mM sodium fluoride) and incubation at 37° C. for 20 minutes. The incubated solution was chilled by the addition of ice-cold Wash Buffer (final concentration 1×PBS and either 4% bovine serum albumin (BSA) or 4% heat-inactivated fetal bovine serum (FBS), supplemented with 1×PIC), and the samples were centrifuged at 300 g for 6 minutes at 4° C.). If the lysis was incomplete, samples were retreated with 0.1% Triton X-100 at 37° C. for 10 to 20 minutes. After removal of the supernatant, cells were washed twice in ice-cold Wash Buffer supplemented with 1×PIC and permeabilized by the addition of pre-chilled 80% methanol while simultaneously vortexing the tube and incubated on ice for 10 minutes. Methanol was removed by centrifugation, the pellet was washed with ice-cold 1×PBS, and non-specific binding was blocked by the addition of ice-cold Wash Buffer supplemented with 1×PIC and incubation at 4° C. for a minimum of 30 minutes.

To stain cells with fluorescent conjugated monoclonal antibodies, the incubated solution was centrifuged to remove the supernatant and the pellet was incubated for 60 minutes at 4° C. with an antibody staining solution including 0.25 μg to 0.50 μg of pS6-Pacific Blue, pERK-ALEXA 488, pSTAT5-ALEXA 647, CD34-PE, CD45-PerCP, and CD117-PC7. Samples were washed three times with ice-cold Wash Buffer supplemented with 1×PIC, resuspended in 0.5 mL ice-cold Wash Buffer supplemented with 1×PIC including 0.5 μg/mL DAPI, and analyzed by flow cytometryusing a BD LSR II Flow Cytometer, equipped with a High Throughput Sampler (HTS).

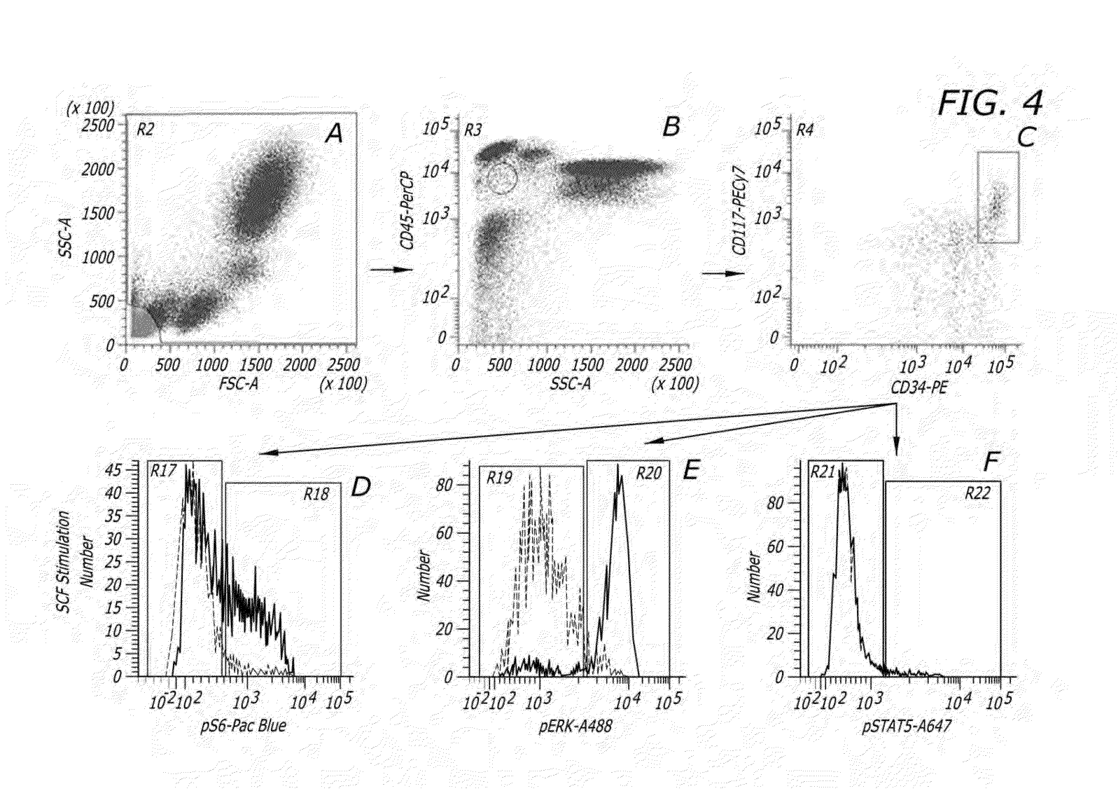

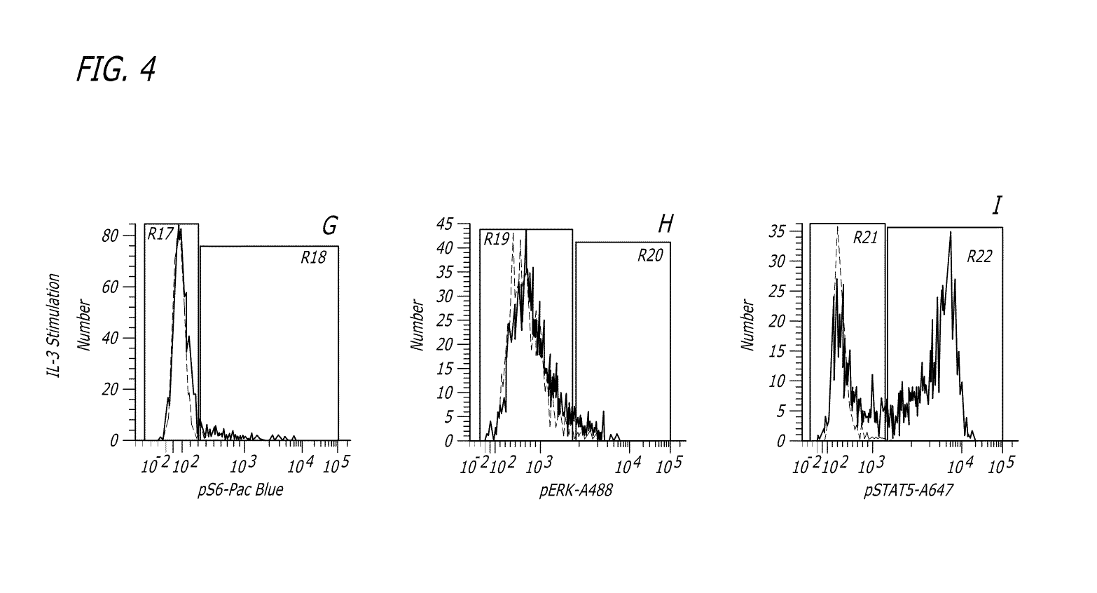

A gating strategy is shown in FIG. 4. First, doublets, aggregates, and debris were excluded by gating on single cell events in the SSC-Area versus SSC-Height histogram. Then, in events passing through this gate, debris near the origin of the FSC-Area versus SSC-Area plot (gray in FIG. 4A) was excluded by drawing a gate around an area encompassing, primarily, lymphocytes, monocytes, and granulocytes (LMG, black in FIG. 4A). Next, in cells passing through the LMG gate, the blast region (circle in FIG. 4B) was identified in the CD45 versus SSC-Area histogram (FIG. 4B). From this blast population, CD34+, CD117+ cells were identified in the CD34 versus CD117 plot (boxed area in FIG. 4C). Finally, these cells were further analyzed for cytokine-mediated increases in phosphorylation by constructing gates in the single parameter histograms (for positively and negatively stained events, i.e., for the phosphorylated and non-phosphorylated forms, respectively, of each protein) for pS6 (FIGS. 4D and 4G), pERK (FIGS. 4E and 4H), and pSTAT5 (FIGS. 4F and 4I). The single parameter histograms show the responses typically obtained for SCF-stimulated pS6 (4 min; FIG. 4D), pERK (2 min; FIG. 4E), and pSTAT5 (2 min; FIG. 4F) as well as for IL-3 stimulated pS6 (FIG. 4G), pERK (FIG. 4H), and pSTAT5 (FIG. 41) at 8 min. The dotted lines in each of these histograms represent unstimulated cells at t=0 min. FCS files were processed with WinList 6.0 3D (Verity Software House). Analysis consisted of a gating strategy to monitor cytokine-mediated phosphorylation in CD34+, CD117+ cells, detailed examination of the phosphorylation data from pS6, pERK, and pSTAT5 single parameter plots (by at least three different methods), and graphical depiction of the resulting kinetic profiles with GraphPad Prism version 5.03 for Windows (GraphPad Software, Inc.). Following this gating scheme, data were analyzed by calculating the area under the curve (AUC), the frequency of positive-stained cells, and the median fluorescence intensity (MFI).

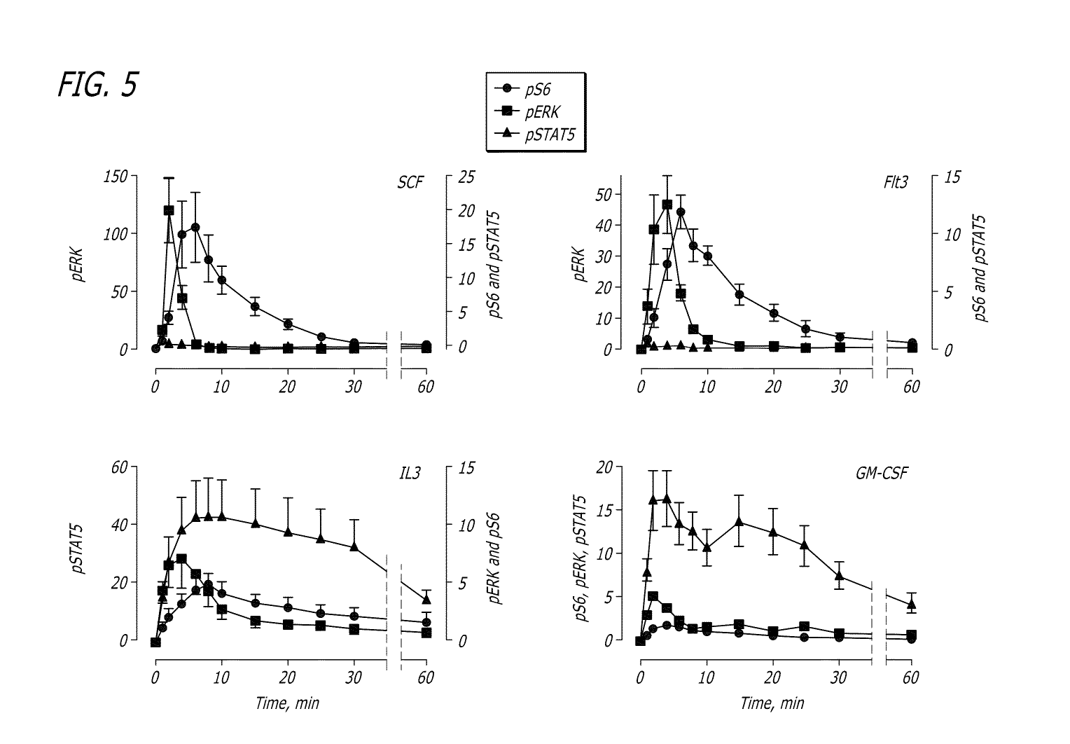

The composite kinetic profiles for SCF-, FL-, IL-3-, and GM-CSF-stimulated phosphorylation of pERK, pS6, and pSTAT5 in CD34+, CD117+ cells from healthy donors are shown in FIG. 5 as mean fold stimulation ±SEM at each time point. In general, SCF and FL were good stimulators of pERK and pS6, but had very little, if any, discernable effect on pSTAT5. In contrast, both IL-3 and GM-CSF were good stimulators of pSTAT5, with IL-3 being the better of the two. In addition, IL3 stimulated pERK and pS6, but at levels less than that observed for SCF and FL. GM-CSF only weakly stimulated pERK and pS6.

SCF-stimulated ERK phosphorylation was rapid (reaching a maximum at tₘₐₓ=2 min), transient (the response interval was 8.7 min with a range of 8 to 10 min), and, of the three phosphoproteins, showed the greatest response to SCF (amplitude=120 with a range of 17 to 257)(FIG. 5, Table 8). In comparison, phosphorylation of S6 was less robust (at its maximum, approximately 6-fold less than pERK) and less rapid (tₘₐₓ=5.8; range=4 to 8 min), but longer in duration (interval=28 min; range=20 to 30 min). Furthermore, SCF had little or no effect on STAT5 phosphorylation in these cells, showing little or no response above control. (Table 8).

TABLE 8
Cytokine-Stimulated Phosphorylation in CD34⁺, CD117⁺ Cell Populations from Healthy Donor Samples

| | pERK | | | pS6 | | | pSTAT5 | | |
|---|---|---|---|---|---|---|---|---|---|
| | | tₘₐₓ | RI | | tₘₐₓ | RI | | tₘₐₓ | RI |
| Cytokine | Amp¹ | (min)² | (min)³ | Amp. | (min) | (min) | Amp. | (min) | (min) |
| SCF | 120 ± 28 | 2.0 ± 0 | 8.7 ± 0.3 | 18 ± 5.1 | 5.8 ± 0.5 | 28 ± 1.2 | 0.8 ± 0.2 | 2.4 ± 0.4 | Variable |
| FL | 51 ± 11 | 3.4 ± 0.4 | 18 ± 1.5 | 12 ± 1.4 | 6.3 ± 0.3 | 37 ± 6.0 | 0.8 ± 0.2 | 4.3 ± 1.9 | Variable |
| IL-3 | 6.9 ± 2.4 | 5.3 ± 2.2 | Variable | 4.2 ± 1.0 | 7.4 ± 0.6 | Variable | 38 ± 12 | 11 ± 2.8 | >60 |
| GM-CSF | 4.5 ± 0.6 | 2.5 ± 0.4 | Variable | 1.8 ± 0.3 | 6.6 ± 1.4 | Variable | 17 ± 2.9* | 9.9 ± 2.9 | >60 |

¹Amp, peak amplitude of fold stimulation, Fold stimulation was calculated by estimating the AUC of both positive-stained and negative-stained cells, then expressing this as the ratio of positives to negatives at each t = n, and, finally, as the fold change of positives/negatives at t = n over the baseline ratio at t = 0 min).
²tₘₐₓ, peak time.
³RI, response interval.
*For biphasic responses, the larger of two values was used in the calculation of the statistic.

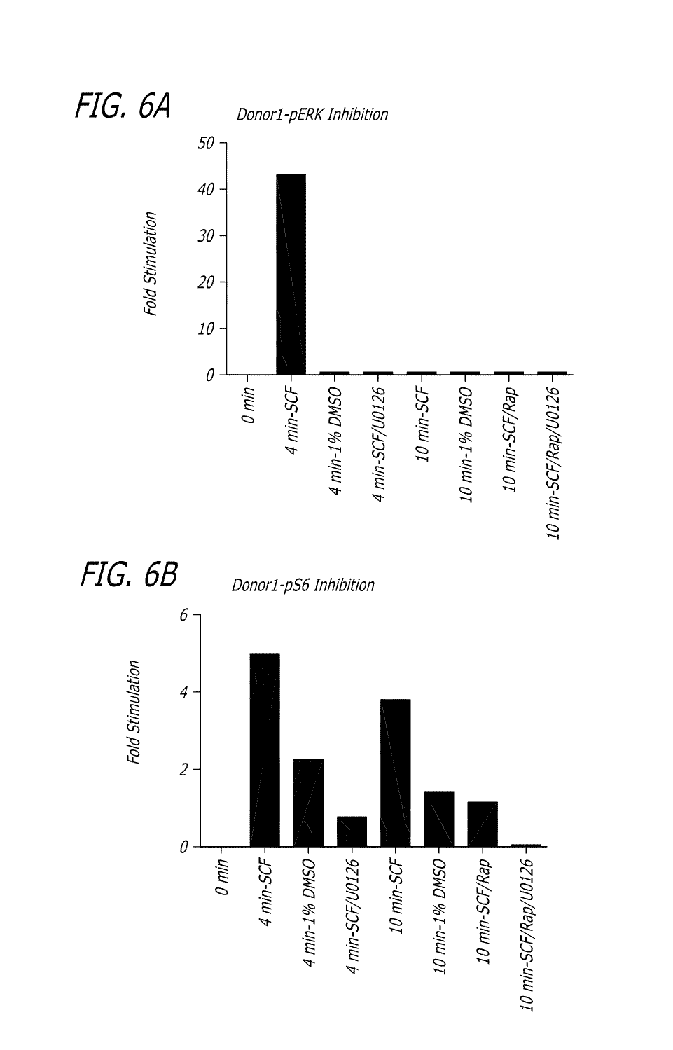

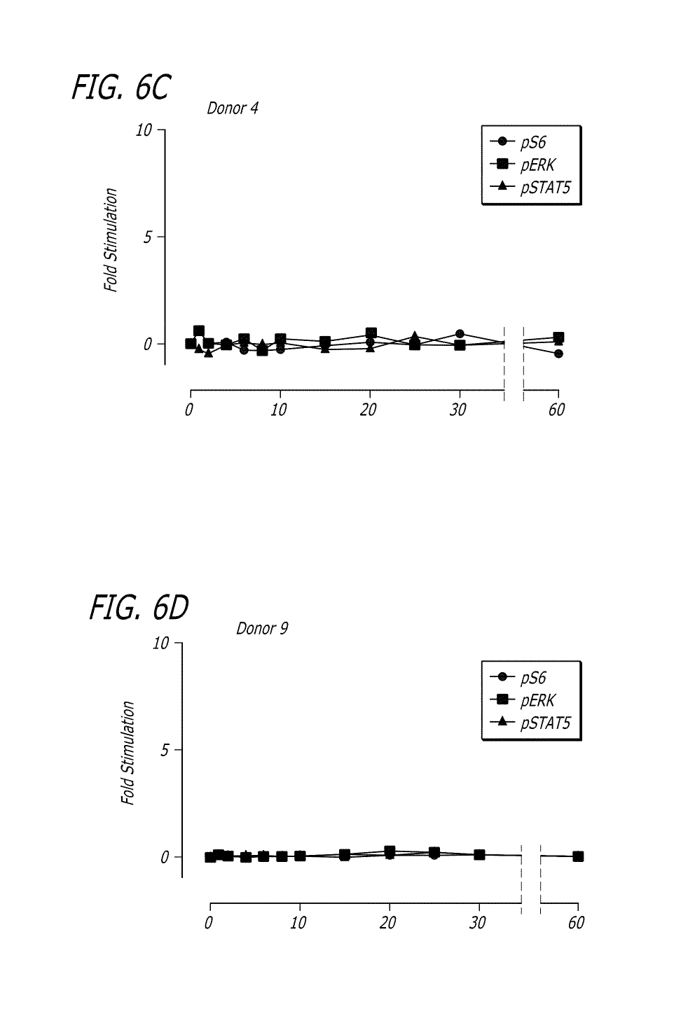

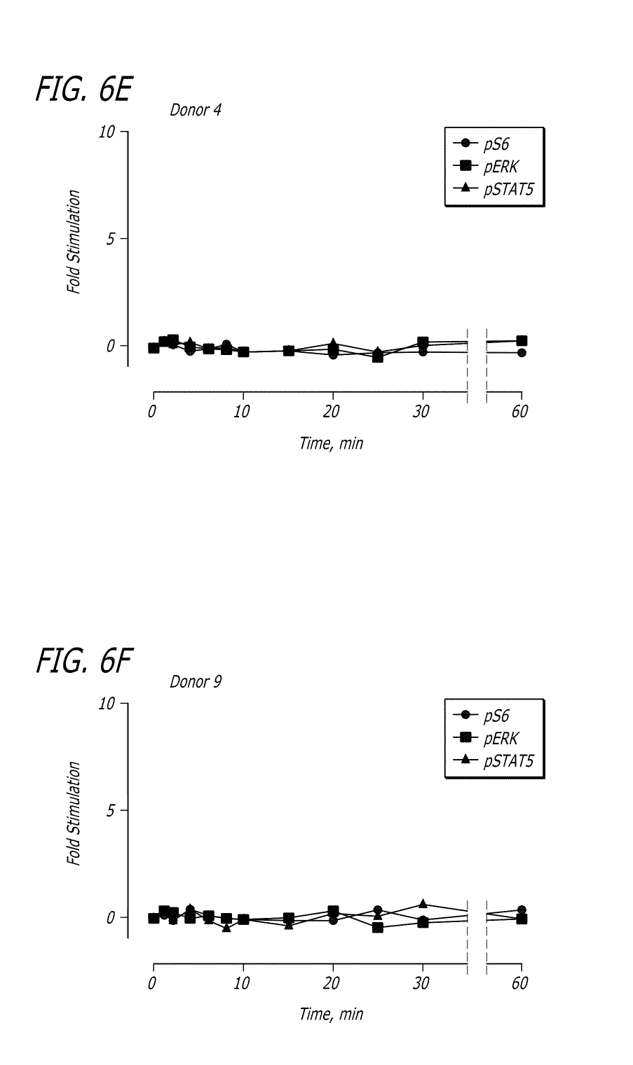

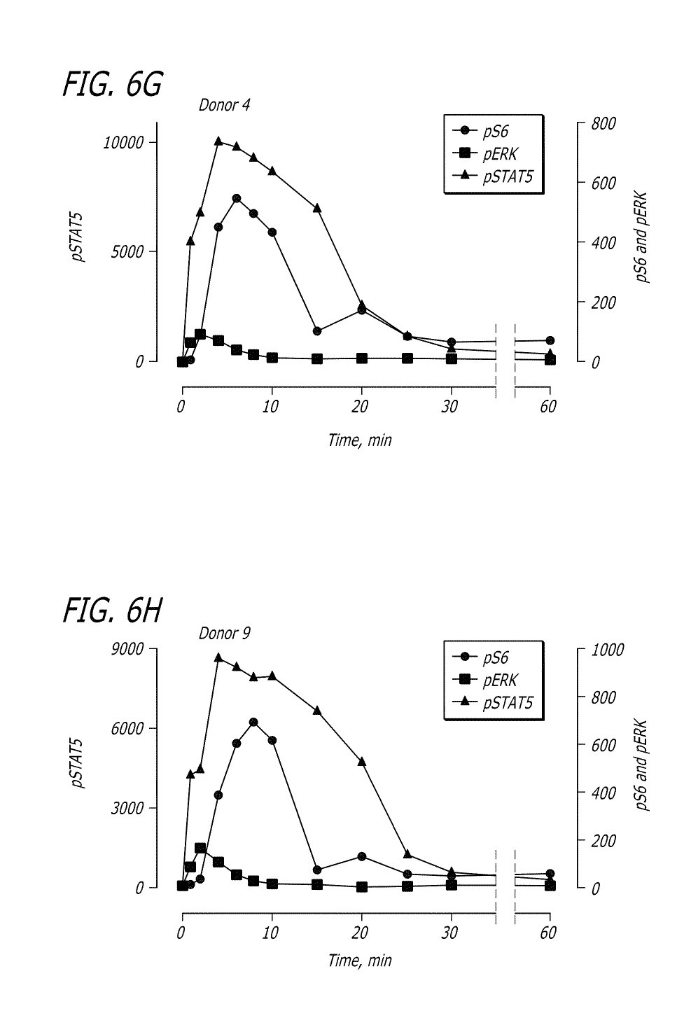

Inhibitor studies with U0126 and rapamycin to block MEK and mTOR, respectively, demonstrated the specificity of the SCF-stimulated response. U0126 completely inhibited SCF-stimulated ERK phosphorylation (FIG. 6A), and, furthermore, partially blocked SCF-stimulated S6 phosphorylation at 4 min (FIG. 6B). In addition, treatment with rapamycin partially blocked S6 phosphorylation at 10 min, and, when combined with U0126, completely inhibited S6 phosphorylation (FIG. 6B). Partial inhibition of S6 phosphorylation by each inhibitor separately, as well as complete inhibition when the two inhibitors were combined, suggest that both MEK and mTOR pathways contribute to SCF-stimulated S6 phosphorylation in CD34⁺, CD117⁺ cells. In addition, the specificity of SCF-mediated phosphorylation in CD34⁺, CD117⁺ cells is demonstrated further by the lack of SCF's effect in lymphocytes, cells which are not known to possess receptors for SCF and thus would not be expected to show any detectable increase in phosphorylation. (FIGS. 6C and 6D).

FL-stimulated phophoprotein profile was similar to that observed for SCF, but with some distinguishing nuances in ERK, but not in S6 or STAT5, phosphorylation (FIG. 5, Table 8). In comparison to SCF, FL-stimulated phosphorylation of ERK was less robust (amplitude=51; range=20 to 103), less rapid (reaching a maximum at tₘₐₓ=3.4 min), but lasted longer (18 min with a range of 15 to 25 min). For samples exhibiting the greatest FL stimulation, the pERK response was approximately 2.5-fold less than those samples maximally stimulated with SCF. Contrasting ERK phosphorylation, the trends observed for FL-stimulated S6 phosphorylation were similar to those observed for SCF treatment: the amplitude was 12 with a range of 5 to 15; tₘₐₓ was 6.3 min with a range of 6 to 8 min; and the response interval was 37 min with a range of 25 to 60 min. Like SCF, FL did not stimulate STAT5 phosphorylation. Like SCF, FL elicited no response in lymphocytes, demonstrating the specificity of this cytokine in CD34⁺, CD117⁺ cells (FIGS. 6E and 6F).

The phosphoprotein profile of IL-3-stimulated pERK, pS6, and pSTAT5 (FIG. 5, Table 8) is very different from the SCF- and FL-mediated profiles: STAT5 phosphorylation was rapid and robust, with a slow, gradual decay over the later 80% of the time course, whereas phosphorylation of both ERK and S6 was muted. IL-3-stimulated pSTAT5 reached its maximum (amplitude =38 with a range of 7 to 110) at 11 min (range=6 to 30 min) and slowly decayed to approximately 30% to 50% of its maximal value at 60 min. In contrast, the amplitudes of pERK and pS6 were relatively small (6.9 and 4.2, respectively) in comparison to both IL-3 stimulated pSTAT5 as well as SCF- and FL-stimulated pERK and pS6.

Of the cytokines tested, the profile mediated by GM-CSF was the most variable (FIG. 5, Table 8). In general, GM-CSF-stimulated phosphorylation of STAT5 was robust, reaching a maximum (amplitude=17 with a range of 7.6 to 30) at approximately 10 min (range=2 to 20 min), and then decayed slowly throughout the remainder of the time course; four of the eight samples exhibited some bimodality in their decay. In addition, GM-CSF very weakly stimulated pERK and pS6 (amplitudes=4.5 and 1.8, respectively), and, of the cytokines tested, was the weakest stimulator of these phophoproteins. As a point of comparison, the ability of GM-CSF to stimulate pS6, pERK, and pSTAT5 was also evaluated in monocytes (FIG. 6G and 6H). GM-CSF very strongly stimulated both pSTAT5 and pS6: the maximal fold stimulation of pSTAT5 was approximately 10,000 or about 500 times the level observed in CD34⁺, CD117⁺ cells. Similarly, the maximal fold stimulation of pS6 was approximately 500 or about 100 times the level observed in CD34⁺, CD117⁺ cells. In contrast, pERK was moderately stimulated in monocytes; the maximal fold stimulation was approximately 100. As this comparison demonstrated, GM-CSF is a very weak effector in CD34⁺, CD117⁺ cells.

In conclusion, the SCF- and FL-mediated profiles for pS6, pERK, and pSTAT5 in CD34⁺, CD117⁺ cells from NBM are, in general, very similar, with SCF and FL stimulating rapid, transient phosphorylation of ERK, less rapid but longer-lived phosphorylation of S6, and little, if any, phosphorylation of STAT5. Notably, both KIT and FL are class III RTKs, and thus share some structural and functional homology, which may explain, in part, their similar profiles, as a common preference (determined at the level of the receptor) for signaling via PI3K-AKT and RAS-MAPK over JAK-STAT. A comparable argument can be made for the IL-3- and GM-CSF-mediated profiles in CD34⁺, CD117⁺ cells from healthy donor samples. In this case, IL-3 and GM-CSF stimulated rapid and relatively sustained phosphorylation of STAT5, but only weak to moderate phosphorylation of S6 and ERK. Receptors for these ligands share a common signal transduction subunit, which, in a manner analogous to KIT and FLT-3, may signal preferentially via JAK-STAT over PI3K-AKT and RAS-MAPK.

### Example 5

### Stability of Stimulated Signals

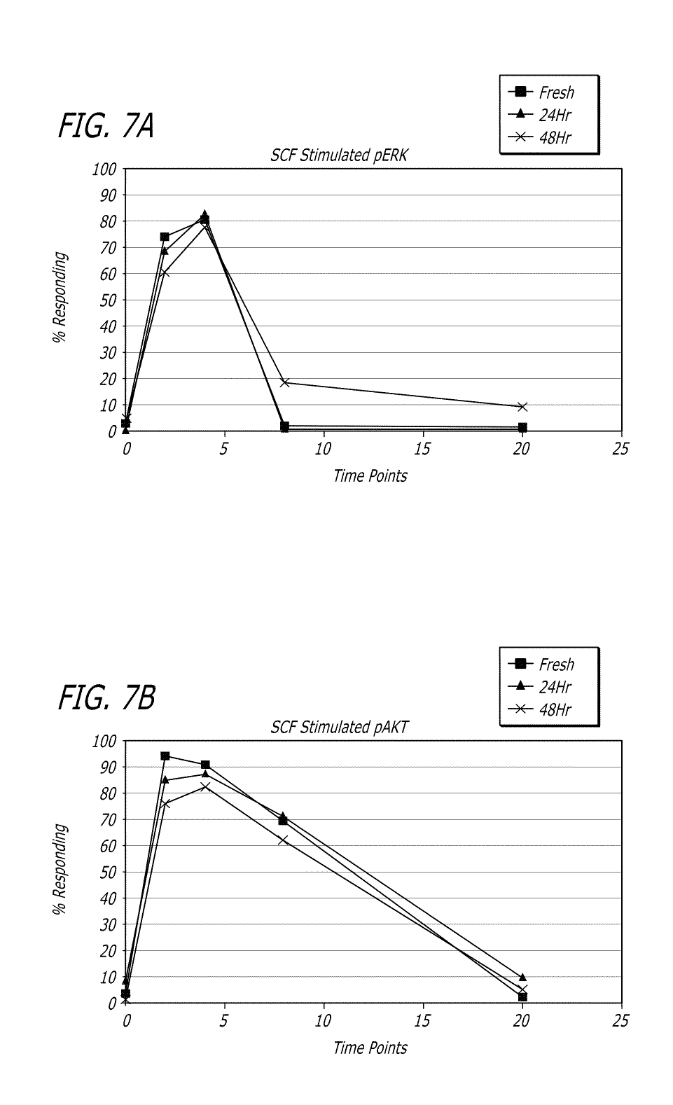

The stability of stimulated signals in normal bone marrow samples was tested using normal bone marrow samples stimulated with SCF for 0, 2, 4, 8, or 20 minutes. The pERK or pAKT signaling in these samples was measured again after 24 hours or 48 hours of sample storage at room temperature past the initial stimulation time period. The percent positive responding CD34⁺, CD117⁺ cells were plotted against the stimulation time points. The results are shown in FIG. 7A (pERK signaling) and FIG. 7B (pAKT signaling). It is shown that over a 48-hour time period after the initial stimulation, there was minimal change in signaling profiles.

### Example 6

### Phosphoprotein Activation Profiles in Bone Marrow Samples from AML Individuals

Bone marrow samples from AML patients were processed in the same manner as bone marrow samples from healthy donors as described in Example 4, except that there was about a 24 hour delay between collection of the sample and its use in subsequent experimentation.

AML1 was from a 71-year-old woman, who was diagnosed 3 years prior with AML subtype M4 secondary to chemotherapy for breast cancer; prior therapy is unknown. CBC at presentation was 40.7×10⁶ WBC/mL, 1.78×10⁹ RBC/mL, 11×10⁶ PLT/mL, HGB of 5.7 g/dL, HCT of 19.7%, and 60% circulating blasts. Bone marrow consisted of 58% blasts. Cytogenetics showed t(9;11)(p22;q23) translocation, which is consistent with therapy-related AML. Flow cytometry showed two abnormal populations: First, a predominant cell population characterized by CD13^{+ (partial)}, CD71^{+ (partial)}, HLA-DR^{+ (partial)}, CD11b^{+ (partial)}, CD15^{+ (partial)}, CD16^{+ (partial)}, CD7⁻, CD56⁻, CD34^{+ (partial)}, CD117^{+ (heterogeneous expression)}, and CD38⁺. The second population consisted of atypical monocytes (14% blasts), which were CD14⁺, CD13⁺, CD11b⁺, CD15⁺, and CD33^{+ (moderate)}; they also exhibited an abnormally low expression of HLA-DR and CD4. The patient was in remission following induction chemotherapy.

AML2 is from a 29-year-old woman, who was diagnosed 16 months prior with AML subtype M2. The first relapse occurred 2 months after the initial diagnosis; the current specimen represents the second relapse. CBC at presentation was 11.5×10⁶ WBC/mL, 3.91×10⁹ RBC/mL, 26×10⁶ PLT/mL, HGB of 11.3 g/dL, HCT of 34.2%, and rare (<1%) circulating blasts. Bone marrow consisted of 44% blasts. Cytogenetics was normal. Flow cytometry showed blasts characterized by CD13⁺, CD14⁻, HLA-DR^{dim}, CD22^{dim/negative}, CD33^{+ (moderate)}, CD11b^{dim}, CD15^{dim}, CD117⁺, CD34^{+ (partial)}, CD4^{dim}, and CD38⁺, and were partially myeloperoxidase positive (5%). The patient was in remission following mitoxantrone and etoposide therapy, and was awaiting a cord blood transplant.

AML3 is from a 76-year-old female, who came to her clinician's office for allergy testing. Two months earlier she had a normal CBC. The subsequent diagnosis was AML subtype M4Eo. CBC at presentation was 19.6×10⁶ WBC/mL, 2.46×10⁹ RBC/mL, 29×10⁶ PLT/mL, HGB of 7.6 g/dL, HCT of 23.1%. Peripheral blood differential revealed 4% PMNs, 24% lymphocytes, 40% monocytes, 2% eosinophils, and 30% blasts. Bone marrow consisted of 82% blasts (including promonocytes). Cytogenetics showed t(16;16). Flow cytometry showed approximately 21% CD34⁺ cells and approximately 30% CD14⁺ (monocytic) cells in the bone marrow.

AML4 is from a 20-year-old female with acute monoblastic leukemia with maturation (FAB-M5b) and with CNS complications. WBC at presentation was 111×10⁶ WBC/mL. Bone marrow consisted of 88.5% blasts; blasts were CD34 and CD117 negative. Cytogenetic examination was normal. Molecular studies showed a FLT-3 internal tandem duplication with an allelic ratio of 0.87.

AML5 is from a 63-year-old female with acute myelogenous leukemia without maturation (WHO; FAB-M1). WBC at presentation was 124.3×10⁶ WBC/mL. Bone marrow consisted of 95% blasts; CD34 expression was only partial. Cytogenetic examination was normal. FISH for t(8;21) and rearrangement of 16q22 were negative.

To assess the signal transduction response of AML cell populations after growth factor simulation, the AM1L samples were incubated with SCF, FL, IL-3, and GM-CSF, fixed, permeabilized, and stained as described in Example 4. The cell populations were analyzed by flow cytometry using a gating strategy as described in Example 4 and shown in FIG. 4. Bone marrow from AML2 was processed the same way, except, due to its limited quantity, was only stimulated with SCF for 0, 2, 6, 10, 20, and 30 minutes. Results are expressed as the frequency of positive-stained cells, i.e., frequency=(positive-stained cells)/[(positive-stained cells)+(negative-stained cells)]×100.

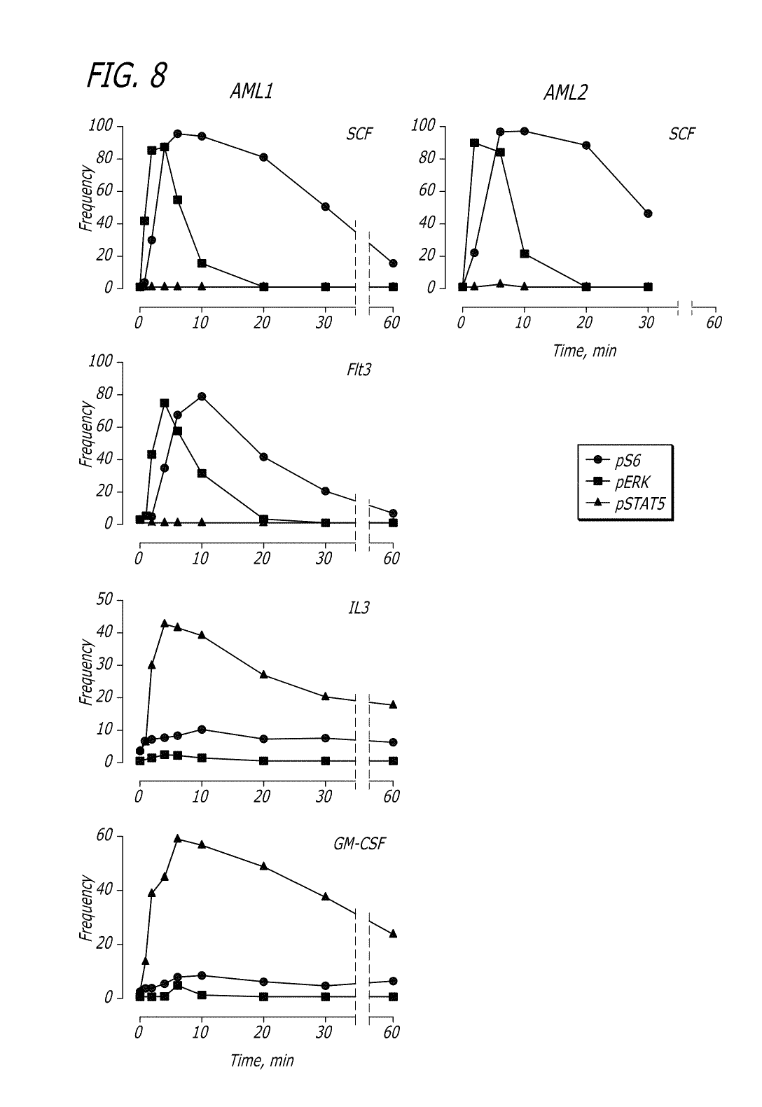

An initial analysis shows that the general trends of SCF-, FL-, IL-3-, and GM-CSF-mediated phosphorylation of ERK, S6, and STAT5 that were observed in healthy donor samples appear to be retained in AML1 and (for SCF treatment only) in AML2 (FIG. 8, Table 9). That is, SCF- and FL-stimulated rapid and transient phosphorylation of ERK, less rapid but longer-lived phosphorylation of S6, and very weak phosphorylation of STAT5 (relative to ERK and S6). In addition, both IL-3 and GM-CSF were strong stimulators of pSTAT5, moderate stimulators of pERK, and weak stimulators of pS6.

TABLE 9
Cytokine-Stimulated Phosphorylation in CD34⁺, CD117⁺ Cell Populations from AML
Samples

| | pERK | | | pS6 | | | pSTAT5 | | |
|---|---|---|---|---|---|---|---|---|---|
| | | tₘₐₓ | RI | | tₘₐₓ | | | tₘₐₓ | |
| Cytokine | Amp¹ | (min)² | (min)³ | Amp. | (min) | RI (min) | Amp. | (min) | RI (min) |
| SCF | 1600 | 4 | 30 | 1100 | 6 | 30 | 32 | 60 | UD* |
| SCF⁴ | 1300 | 2 | 20 | 620 | 10 | 30 | 2 | 2 | 6 |
| FL | 460 | 4 | 30 | 140 | 10 | 30 | 2 | 2 | 2 |
| IL-3 | 5 | 6 | 10 | 1 | 10 | 20 | 150 | 6 | >60 |
| GM-CSF | 6 | 6 | 10 | 1 | 10 | 20 | 110 | 6 | >60 |

¹Amp, peak amplitude of fold stimulation, Fold stimulation was calculated by estimating the AUC of both positive-stained and negative-stained cells, then expressing this as the ratio of positives to negatives at each t = n, and, finally, as the fold change of positives/negatives at t = n over the baseline ratio at t = 0 min).
²tₘₐₓ, peak time.
³RI, response interval.
⁴This SCF experiment was done on AML2 samples, all other results are based on AML1 samples.
*UD, undetermined, in this particular sample, the level of pSTAT5 increased throughout the time course reaching a maximum at 60 min without evidence of decay.

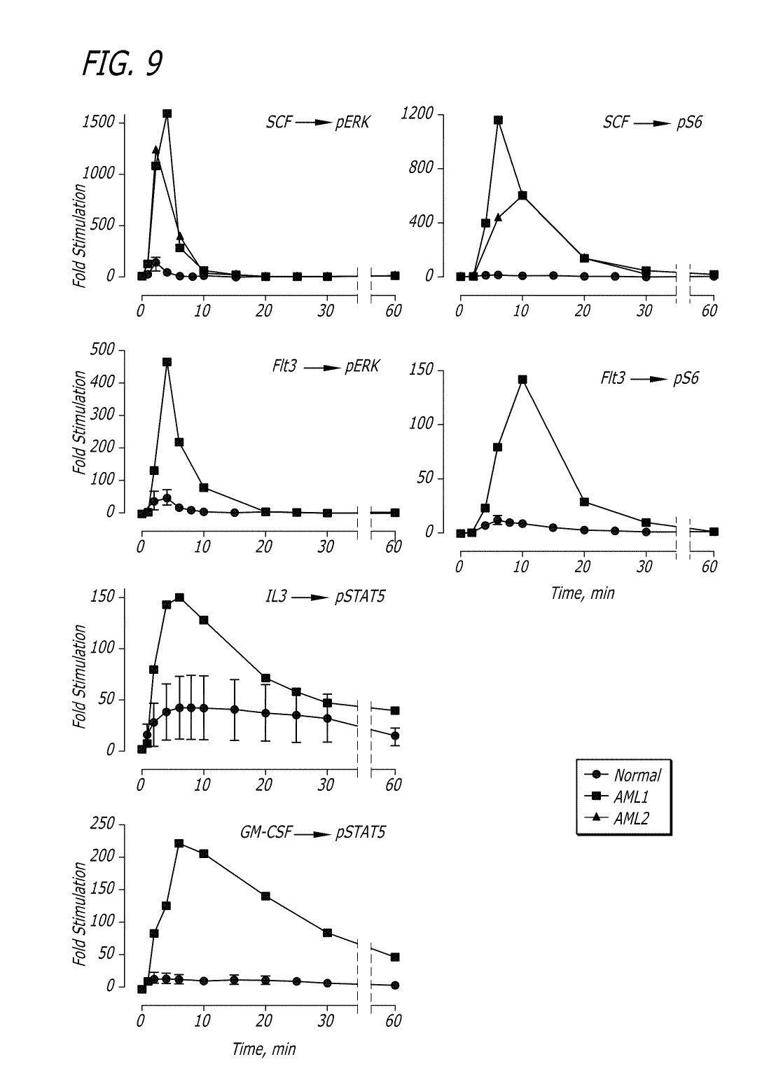

However, some striking differences between samples from AML1, AML2, and the healthy donors were observed (FIG. 9, Table 9). For example, SCF-stimulated pERK in AML1 and AML2 and FL-stimulated pERK in AML1 were approximately 10-fold greater, SCF-stimulated pS6 in AML1 and AML2 were 30- to 60-fold greater, and IL-3- and GM-CSF-stimulated pSTAT5 were 4- to 6-fold greater than the corresponding responses in healthy donor samples (FIG. 9).

Additionally, for SCF-stimulated pERK (in AML1 but not AML2) the time to maximum response was later (tₘₐₓ=4 min) and the response interval (for both AML1 and AML2) was longer (20 to 30 min), whereas, for FL-stimulated pERK the time to maximum response was approximately the same (tₘₐₓ=4 min) but the response interval was longer (30 min)(Compare Table 8 to Table 9). As another example, for SCF-stimulated pS6 (in AML2 but not AML1) and for FL-stimulated pS6 the times to maximum response were later (tₘₐₓ=10 min), but the response intervals were approximately the same (30 min). For IL-3- and GM-CSF-stimulated pSTAT5 both peak times and response intervals were about the same (Compare Table 8 to Table 9). In general, then, SCF and FL increased the response amplitude, peak time, and response interval of pERK; whereas SCF and FL increased just the response amplitude and peak time of pS6, but not the response interval; while IL-3 and GM-CSF increased only the response amplitude but neither the peak time nor the response interval, compared with the corresponding responses in healthy donor samples.

Finally, the shapes of the curves, while similar to those of healthy donor samples were also different, as the decay portions of the curves, for SCF- and FL-stimulated pERK and pS6, and for IL-3- and GM-CSF-stimulated pSTAT5, were markedly right-shifted with greater slopes in the AML samples (FIG. 9). These data indicate that SCF- and FL-stimulated pS6 (but not pERK) was dramatically elevated in samples from AML patients above the levels observed in samples taken from healthy donors.

### Example 7

### Comparison of SCF-Stimulated pERK and pS6 in Healthy Donor and AML Bone Marrow Samples

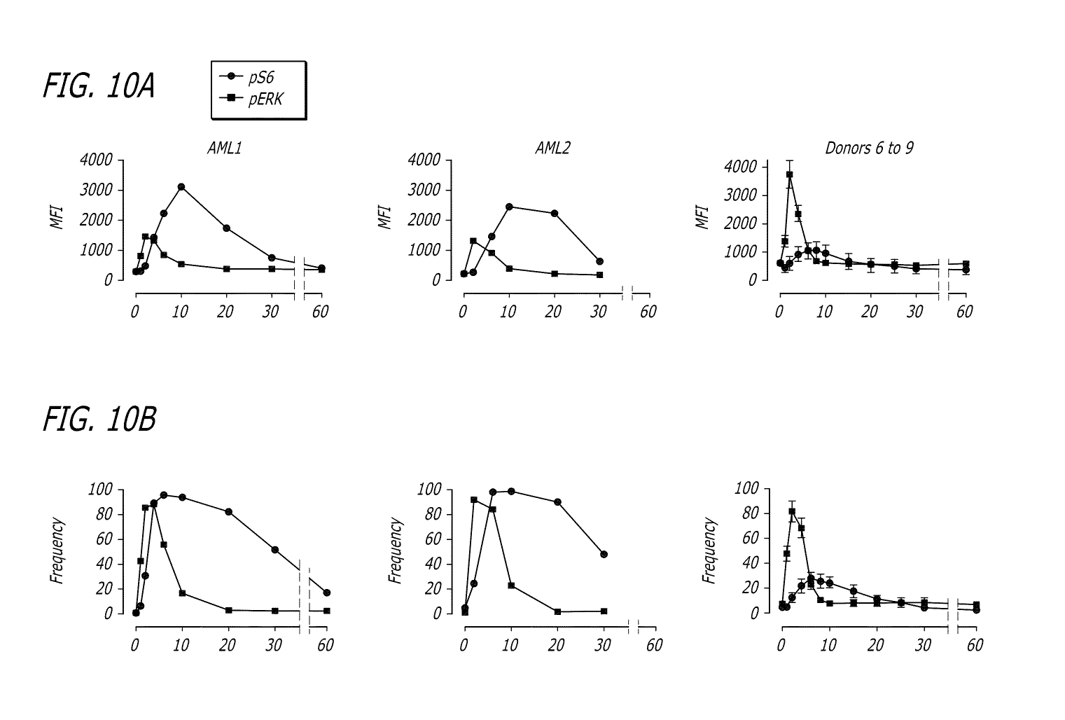

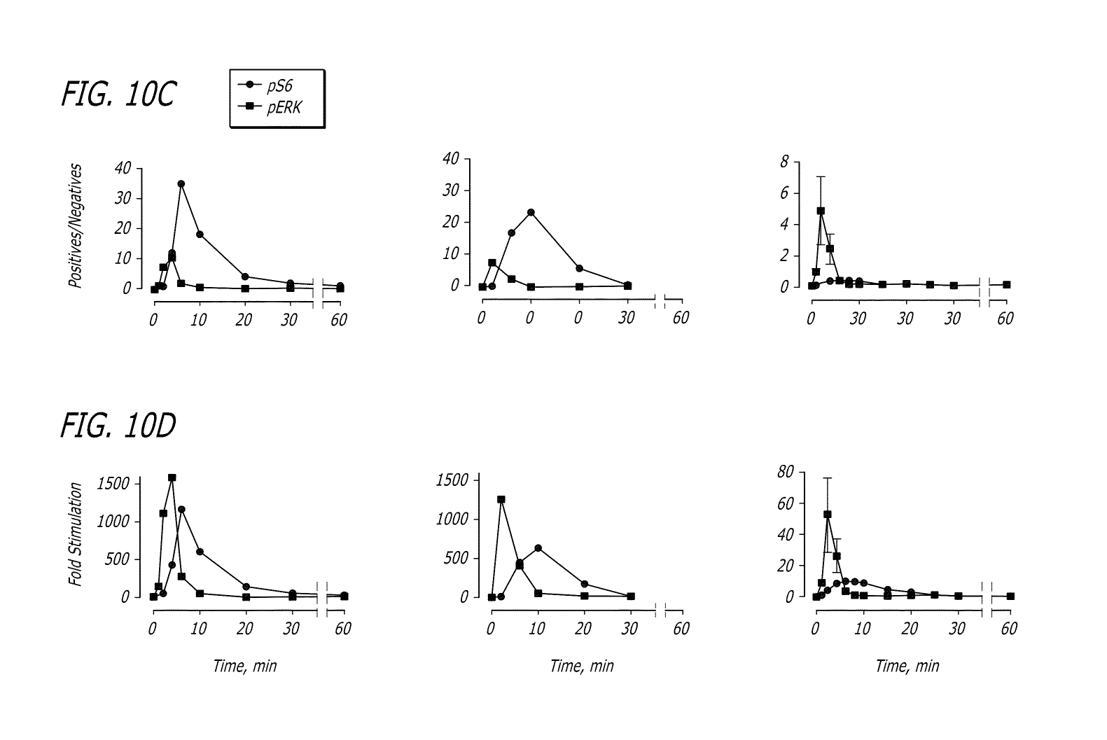

In an attempt to dissect the differences between the pERK and pS6 kinetic profiles in healthy donor and AML bone marrow sampless, SCF-stimulated pERK and pS6 data derived from AML1, ALM2, and healthy donors 6 to 9 were analyzed from four different perspectives: MFI, the frequency of positive-stained cells, the ratio of positive to negative cells, and fold stimulation (FIG. 10 and Table 10). The ratio of positives to negative cells was calculated by estimating the AUC of both positive- and negative-stained cells at each t=n, and then expressing this as the ratio of positives to negatives. The fold stimulation was the fold change of the positive/negative ratio at t=n over the baseline ratio at t=0 min. Notably, in healthy donor samples, when analyzed by each of these four methods, the effect of SCF was much greater on pERK than pS6. However, this was not the case in the AML samples.

First, in analyzing MFI, the maximal MFI increased 2-to 3-fold for pS6 and decreased 0.35- to 0.41-fold for pERK in the AML samples compared with healthy donor samples. Additionally, the [(pERK)ₘₐₓ:(pS6)ₘₐₓ]_{MFI} ratio decreased from 3.6 in healthy donor samples to about 0.5 in the AML samples. Thus, the trend observed in healthy donor samples, where pERK showed the greater response to SCF, and not pS6, was reversed in the AML samples. Second, at its maximum, the frequency of positively stained cells in the AML samples was approximately 90% and 97% for pERK and pS6, respectively, compared with 81% and 26% for pERK and pS6, respectively, in healthy donor samples. Thus, although the frequency of pERK-stained cells was comparable between the AML and healthy donor samples, the pS6 frequency increased almost 4-fold in the AML samples. Third, when analyzing the ratio of positive- to negative-stained cells, the trend was similar to that observed for MFI; namely, it was reversed between the healthy donor and AML samples. The positive to negative cell ratio in the AML samples increased 50- to 80-fold and only 1.5- to 2.0-fold for pS6 and pERK, respectively, compared with healthy donor samples; and the ratio, [(pERK)ₘₐₓ:(pS6)ₘₐₓ]_{POS/NEG}, decreased from 11 in healthy donor samples to about 0.3 in the AML samples. Finally, fold stimulation was dramatically increased in the AML samples compared with healthy donor samples for pERK and pS6, the increases were 25- to 30-fold and 60- to 110-fold, respectively, over the levels observed in healthy donor samples. In general, this multifaceted approach indicates that SCF-stimulated pS6 phosphorylation was substantially amplified compared to pERK in the AML samples and compared to both pS6 and pERK in healthy donor samples.

The value of analyzing the data by several different methods became apparent when the phosphoprotein profiles of AML bone marrows were compared with the profiles of NBMs. The SCF-stimulated pERK and pS6 data were analyzed for MFI_{Total}, frequency of positive-stained cells, ratio of positive- to negative-stained cells, and fold stimulation over basal phosphorylation (at t=0 min). In healthy donor samples each of these analyzes returned the same trend (i.e., pERK>pS6). However, in the AML samples this was not the case: Frequency and fold stimulation showed the pERK response was approximately equal to or greater than the pS6 response, whereas MFI and positives/negatives showed very clearly that pS6 was greater than pERK (by approximately 2- and 3-fold, respectively). In addition, the positives/negatives ratio of pERK in the AML samples was approximately equivalent to this ratio in NBM, whereas the MFI of pERK in the AMLs was approximately 2.5 fold less than the level observed in NBM. Furthermore, the basal phosphorylation of S6 and ERK determines the relationship between the positives/negatives and the fold stimulation plots: Essentially, the basal phosphorylation of S6 was greater than the basal level of ERK in the AML samples, resulting in a fold stimulation plot that showed a greater cytokine-stimulated response for pERK in relation to pS6. However, this analysis was given less weight among the four, since it involves division by relatively small, but approximately similar values. Collectively, when the data from these analyzes were taken together, the MFI and frequency plots suggest that SCF preferentially signals via phosphorylation of S6 rather than ERK in the AML samples.

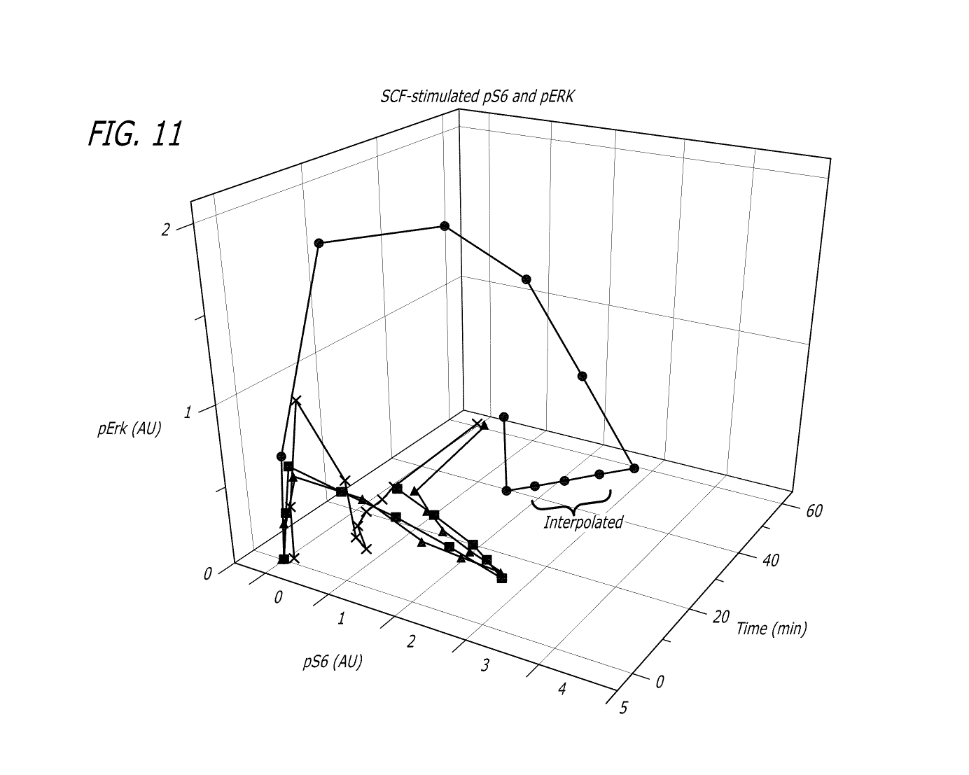

The initial phosphorylation rates (kinetics) in pERK plus pS6 “space” (Arbitrary Units) is essentially identical for composite data of healthy donor samples and all 3 different AML samples (FIG. 11). Duration in pERK+pS6 space is similar for AML1 and 2; both are significantly different than normal. AML3 duration is significantly different from AML1 and 2 (and different from healthy donor). Aberrant signal transduction pathway activity is seen in the all AML samples compared to healthy donor samples. The signaling pathway(s) can be defined that are aberrant in AML samples compared to normal samples. AML thus can be detected by the presence of an aberrant signaling signature as opposed to normal signaling. Definition of an aberrant signaling signature then identifies targets for therapeutic intervention.

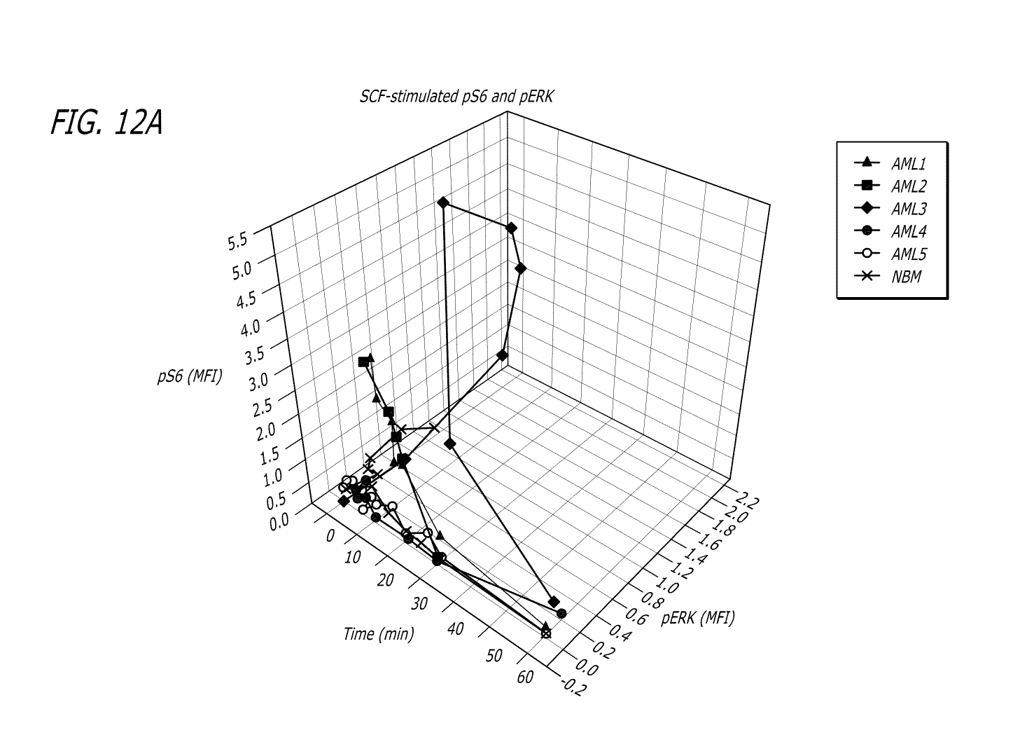

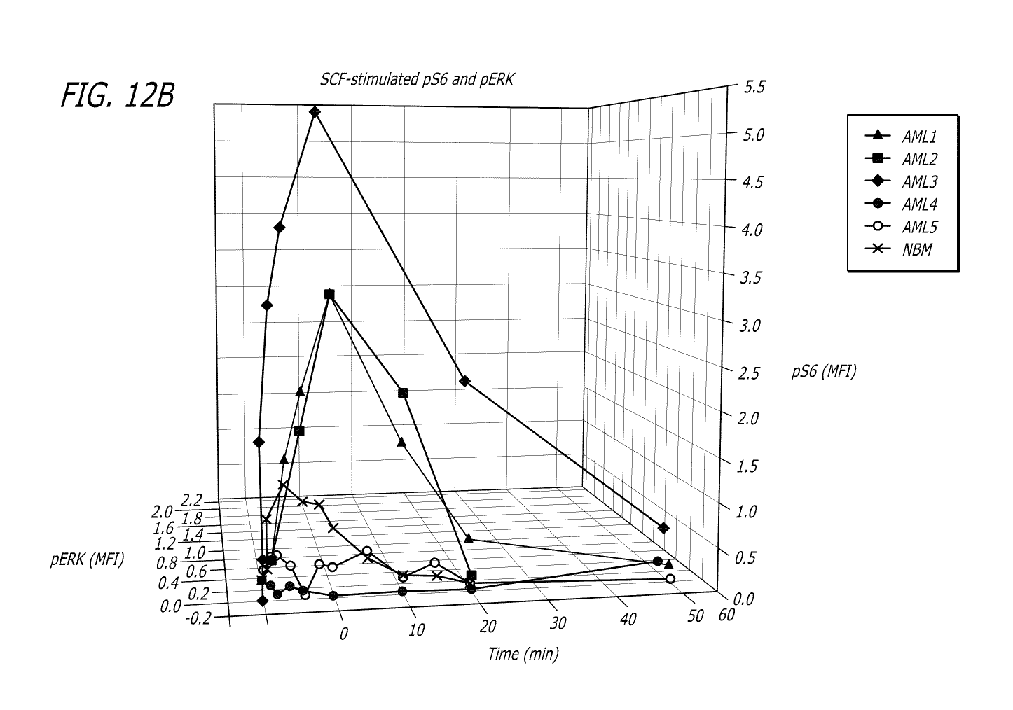

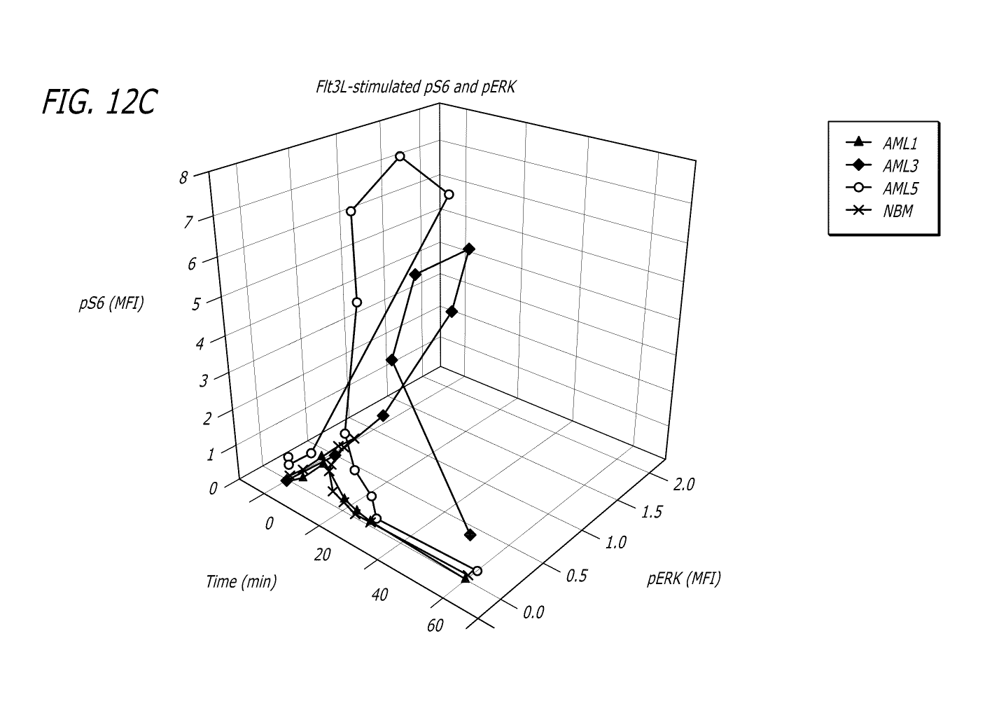

FIG. 12 shows the composite data of healthy donor samples, the data from three AML samples shown in FIG. 13, and the data from two additional AML samples. All AML samples are distinct in the amplitude and duration of signal transduction activity from healthy donor samples (FIG. 14). The signaling profiles seen in AML1 and AML2 are similar to each other while being distinctly different from normal. AML3, AML 4 and AML 5 are different from AML 1 and AML2 while being distinctly different from normal in amplitude and duration of ERK and S6 responses. The signaling profiles show that the AMLs can be classified according to their signaling responses, providing a classification scheme that is different from the FAB classification scheme.

In conclusion, these data indicate 1) that SCF-, FL, IL-3, and GM-CSF-stimulated pS6, pERK, and pSTAT5 kinetic profiles in CD34⁺, CD117⁺ cells from normal, healthy, adult bone marrow were both distinctive and reproducible (regarding specific cytokine-elicited responses); 2) that analysis of bone marrow from two AML patients showed strikingly similar SCF-mediated responses for pERK and pS6, even though the samples were subtyped differently, and further showed markedly elevated cytokine-mediated phosphorylation in comparison with the levels observed in healthy donor samples; 3) that a multi-analytic approach may be necessary to fully uncover the differences (both major and nuanced) between profiles from normal and AML bone marrows; 4) consequently, that baseline phosphoprotein kinetic profiles from normal tissue are essential to understand the comparable profiles from diseased tissue; and 5), that an aberrant signaling signature identifies targets for therapeutic intervention. As this research continues to evolve with advancements in automated sample handling, monoclonal antibody production, flow cytometry, and data processing, the information garnered, as studies from other laboratories already suggest, will contribute to significant improvements in diagnosis and therapy in diseases like AML.

TABLE 10
Comparison of MFI, frequency, positives over negatives, and fold stimulation from normal and AML bone marrows.

| | AML1 | | | AML2 | | | Donors 6 to 9* | | | AML to NBM ratios | | | |
|---|---|---|---|---|---|---|---|---|---|---|---|---|---|
| | | | pERK | | | pERK | | | pERK | (pERK)_{AML1} | (pS6)_{AML1} | (pERK)_{AML2} | (pS6)_{AML2} |
| Parameter | pERK | pS6 | pS6 | pERK | pS6 | pS6 | pERK | pS6 | pS6 | (pERK)_{D6-9} | (pS6)_{D6-9} | (pERK)_{D6-9} | (pS6)_{D6-9} |
| MFI | 1500 | 3100 | 0.48 | 1300 | 2400 | 0.54 | 3700 | 1040 | 3.6 | 0.41 | 3.0 | 0.35 | 2.3 |
| Frequency | 88 | 96 | 0.92 | 90 | 97 | 0.93 | 81 | 26 | 3.1 | 1.1 | 3.7 | 1.1 | 3.7 |
| Positives/ | 10 | 35 | 0.29 | 7.4 | 23 | 0.32 | 4.9 | 0.44 | 11 | 2.0 | 80 | 1.5 | 52 |
| Negatives | | | | | | | | | | | | | |
| Fold | 1600 | 1100 | 1.4 | 1300 | 620 | 2.1 | 53 | 10 | 5.3 | 30 | 110 | 25 | 62 |
| Stimulation | | | | | | | | | | | | | |

*Values are the mean of healthy donors 6 to 9.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method for determining a phosphoprotein activation profile in hematopoietic cells, the method comprising the steps of:
a) incubating a test sample comprising hematopoietic cells with a phosphoprotein activator, wherein the hematopoietic cells comprise a phosphoprotein of at least one signal transduction pathway; and wherein the phosphoprotein activator is capable of activating the phosphoprotein of at least one signal transduction pathway present in the hematopoietic cells of the test sample;
b) contacting the test sample with one or more fluorescently labeled capture molecules, the one or more fluorescently labeled capture molecules comprising at least one fluorescently labeled activated phosphoprotein capture molecule capable of binding to the phosphoprotein of at least one signal transduction pathway activated by the phosphoprotein activator; wherein the at least one fluorescently-labeled control capture molecule includes a fluorescently-labeled CD34 capture molecule, a fluorescently-labeled CD45 capture molecule, a fluorescently-labeled CD117 capture molecule, or any combination thereof; and
c) removing an aliquot from the test sample from at least a first incubation time period and a second incubation time period;
d) detecting fluorescence of the one or more fluorescently labeled capture molecules from the aliquot from the at least a first incubation time period and the second incubation time period; wherein the fluorescence of the at least one fluorescently labeled activated phosphoprotein capture molecule detected from the aliquot for the first incubation time period and the fluorescence of the at least one fluorescently labeled activated phosphoprotein capture molecule detected from the aliquot for the second incubation time period determines the phosphoprotein activation profile in a test sample comprising hematopoietic cells.

2. The method of claim 1, wherein in step (d) the fluorescence of the at least one fluorescently labeled control capture molecule detected from the aliquot for the first incubation time period is subtracted from the fluorescence of the at least one fluorescently labeled activated phosphoprotein capture molecule detected from the aliquot for the first incubation time period and the fluorescence of the at least one fluorescently labeled control capture molecule detected from the aliquot for the second incubation time period is subtracted from the fluorescence of the at least one fluorescently labeled activated phosphoprotein capture molecule detected from the aliquot for the second incubation time period in order to determine the phosphoprotein activation profile in a test sample comprising hematopoietic cells.

3. The method of claim 1, wherein the test sample is from a healthy individual.

4. The method of claim 1, wherein the test sample is from an individual having a disease or disorder associated with the at least one signal transduction pathway.

5. The method of claim 4, wherein the disease or disorder associated with the at least one signal transduction pathway is a leukemia.

6. The method of claim 5, wherein the leukemia is an acute myelogenous leukemia, an acute lymphocytic leukemia, a chronic lymphocytic leukemia, a lymphoma, a follicular lymphoma, or a multiple myeloma.

7. The method of claim 1, wherein the test sample is from an individual receiving a targeted inhibitor of the at least one signal transduction pathway.

8. The method of claim 1, wherein the test sample is a sample from bone marrow, a bone, a lymph node, or a cell suspension.

9. The method of claim 1, wherein the hematopoietic cells comprise lymphocytes, hematopoietic progenitor cells, CD34⁺ CD117⁺ cells, CD34⁻ CD117⁺ cells, hematopoietic stem cells, leukemia stem cells, myeloid progenitor cells, granulocytes, or monocytes.

10. The method of claim 1, wherein the phosphoprotein activator is a cytokine.

11. The method of claim 10, wherein the cytokine comprises SCF, FL, IL-3, G-CSF, GM-CSF, or any combination thereof.

12. The method of claim 1, wherein the at least one signal transduction pathway includes a PI3K-AKT pathway, a mTOR pathway, a RAS-MAPK pathway, a JAK-STAT pathway, or any combination thereof.

13. The method of claim 1, wherein the phosphoprotein of at least one signal transduction pathway includes a S6, an ERK, an AKT, a STAT3, a STAT5, or any combination thereof.

14. The method of claim 1, wherein the at least one fluorescently-labeled phosphoprotein capture molecule includes a fluorescently-labeled pS6 capture molecule, a fluorescently-labeled pERK capture molecule, a fluorescently-labeled pAKT capture molecule, a fluorescently-labeled pSTAT3 capture molecule, a fluorescently-labeled pSTAT5 capture molecule, or any combination thereof.

15. The method of claim 1, wherein in step (d) the fluorescence of the one or more fluorescently labeled capture molecules detected from the aliquot for at least a first incubation time period and the fluorescence of the at least one fluorescently labeled control capture molecule detected from the aliquot for the second incubation time period is analyzed as an area under the curve, a frequency of positive stained cells, a ratio of positive stained cells to negative stained cells, a mean fluorescence intensity, a median fluorescence intensity, a mode fluorescence intensity, or the time/duration of a positive response.

16. The method of claim 1, wherein the phosphoprotein activation profile determined in step (d) is indicative of a disease or condition.

17. The method of claim 1, further comprising evaluating the phosphoprotein activation profile determined in step (d) by comparison with a phosphoprotein activation profile determined in a reference sample comprising hematopoietic cells, wherein the reference sample is not incubated with a phosphoprotein activator.

18. The method of claim 17, wherein the reference sample is an aliquot of the test sample or a standardized reference sample.

19. The method of claim 1, further comprising incubating the test sample with an inhibitor prior to incubating the test sample with the phosphoprotein activator, wherein the inhibitor is capable of inhibiting the activation of a phosphoprotein of at least one signal transduction pathway present in the hematopoietic cells of the test sample.

20. The method of claim 19, wherein the inhibitor is UO126, AZD6244, PD0325901, XL518, hypothemycin, anthrax lethal factor, RAF265, PLX4032, XL281, Bay 43-9006, Zarnestra, rapamycin, Ly294002, GDC-0941, or any combination thereof.
